(19) United States
(12) Patent Application Publication
(10) Pub. No.: US 2021/0115116 A1
(43) Pub. Date: Apr. 22, 2021

(54) METHODS FOR TREATING COMPLEMENT-MEDIATED DISEASES AND DISORDERS

(71) Applicant: Bioverativ USA Inc., Waltham, MA (US)

(72) Inventors: Peter Van Vlasselaer, Portola Valley, CA (US); Graham Parry, South San Francisco, CA (US); Nancy E. StagIiano, South San Francisco, CA (US); Sandip Panicker, South San Francisco, CA (US)

(73) Assignee: Bioverativ USA Inc., Waltham, CA (US)

(21) Appl. No.: 16/494,267

(22) PCT Filed: Mar. 14, 2018
(86) PCT No.: PCT/US2018/022462

§ 371 Date: Sep. 13, 2019

Related U.S. Application Data
(60) Provisional application No. 62/553,059, filed on Aug. 31, 2017.
(60) Provisional application No. 62/471,190, filed on Mar. 14, 2017.

(51) Int. Cl. C07K 16/18; A61K 9/00; A61K 9/08; A61P 7/06; A61P 37/06; A61P 25/02; A61P 25/28

(52) U.S. Cl. CPC C07K 16/18; A61K 9/0019; A61K 9/08; A61P 7/06; A61P 37/06; A61K 2039/545; A61P 25/28; C07K 2317/76; C07K 2317/24; C07K 2317/565; A61P 25/02

(57) ABSTRACT

The present disclosure provides methods of treating a complement-mediated disease or disorder in an individual, and methods of inhibiting activation of complement component C4 in an individual in need thereof. The methods comprise administering to the individual an anti-C1s antibody. The methods also comprise administering an anti-C1s antibody in a fixed dose, e.g., 5.5 g, 6.5 g, or 7.5 g. The methods also comprise administering an effective dose of an anti-C1s antibody to the individual to achieve a minimum serum level of anti-C1s antibody for therapeutic effect.

### CROSS-REFERENCE TO EARLIER FILED APPLICATIONS

The present application claims benefit to U.S. provisional application No. 62/471,190, filed Mar. 14, 2017, and U.S. provisional application No. 62/553,059, filed Aug. 31, 2017, all of which are incorporated herein by reference in their entireties.

### REFERENCE TO SEQUENCE LISTING SUBMITTED ELECTRONICALLY

The content of the electronically submitted sequence listing in ASCII text file (Name: 4159.505PC02_SeqListing.TXT; Size: 24,288 bytes; and Date of Creation: Mar. 13, 2018) filed with the application is incorporated herein by reference in its entirety.

### BACKGROUND OF THE INVENTION

The complement system is a well-known effector mechanism of the immune response, providing not only protection against pathogens and other harmful agents but also recovery from injury. The complement pathway comprises a number of proteins that typically exist in the body in inactive form. The classical complement pathway is triggered by activation of the first component of complement, referred to as the C1 complex, which consists of C1q, C1r, and C1s proteins. Upon binding of C1 to an immune complex or other activator, the C1s component, a diisopropyl fluorophosphate (DFP)-sensitive serine protease, cleaves complement components C4 and C2 to initiate activation of the classical complement pathway. The classical complement pathway appears to play a role in many diseases and disorders.

There is a need in the art for compounds that treat a complement-mediated disease or disorder. There is also a need for compounds that can detect or monitor such disease or disorder. Also needed are methods to produce and use such compounds and compositions thereof.

### BRIEF SUMMARY OF THE INVENTION

The present disclosure provides methods of treating a complement-mediated disease or disorder in an individual, and methods of inhibiting activation of complement component C4 in an individual in need thereof. In some aspects, the methods comprise administering to the individual an anti-C1s antibody in a fixed dose of 5.5 g. In some aspects, the anti-C1s antibody is administered to the individual every other week.

In some aspects, the anti-C1s antibody comprises light chain complementarity determining regions (CDRs) of an antibody light chain variable region comprising amino acid sequence SEQ ID NO:7 and heavy chain CDRs of an antibody heavy chain variable region comprising amino acid sequence SEQ ID NO:8.

In some aspects, the anti-C1s antibody is humanized. In some aspects, the humanized antibody comprises a humanized light chain framework region and/or a humanized heavy chain framework region.

In some aspects, the anti-C1s antibody comprises: i) a light chain variable region comprising a complementarity-determining region (CDR) comprising a CDR-L1 having the amino acid sequence of SEQ ID NO:1, a CDR-L2 having the amino acid sequence of SEQ ID NO:2, a CDR-L3 having the amino acid sequence of SEQ ID NO:3; and ii) a heavy chain variable region comprising a CDR comprising a CDR-HI having amino acid sequence SEQ ID NO:4, a CDR-H2 having amino acid sequence SEQ ID NO:5, and a CDR-H3 having amino acid sequence SEQ ID NO:6.

In another aspect, the anti-C1s antibody comprises: i) a light chain variable region comprising a complementarity-determining region (CDR) comprising a CDR-L1 having the amino acid sequence of SEQ ID NO:10, a CDR-L2 having the amino acid sequence of SEQ ID NO:11, a CDR-L3 having the amino acid sequence of SEQ ID NO:3; and ii) a heavy chain variable region comprising a CDR comprising a CDR-HI having amino acid sequence SEQ ID NO:12, a CDR-H2 having amino acid sequence SEQ ID NO:13, and a CDR-H3 having amino acid sequence SEQ ID NO:14.

In another aspect, the anti-C1s antibody comprises: a VL region comprising the amino acid sequence set forth in SEQ ID NO:15; and a VH region comprising the amino acid sequence set forth in SEQ ID NO:18.

In another aspect, the anti-C1s antibody comprises: a VL region comprising the amino acid sequence set forth in SEQ ID NO:15; and a VH region comprising the amino acid sequence set forth in SEQ ID NO:19.

In another aspect, the anti-C1s antibody comprises: a VL region comprising the amino acid sequence set forth in SEQ ID NO:15; and a VH region comprising the amino acid sequence set forth in SEQ ID NO:20.

In another aspect, the anti-C1s antibody comprises: a VL region comprising the amino acid sequence set forth in SEQ ID NO:15; and a VH region comprising the amino acid sequence set forth in SEQ ID NO:21.

In another aspect, the anti-C1s antibody comprises: a VL region comprising the amino acid sequence set forth in SEQ ID NO:16; and a VH region comprising the amino acid sequence set forth in SEQ ID NO:18.

In another aspect, the anti-C1s antibody comprises: a VL region comprising the amino acid sequence set forth in SEQ ID NO:16; and a VH region comprising the amino acid sequence set forth in SEQ ID NO:19.

In another aspect, the anti-C1s antibody comprises: a VL region comprising the amino acid sequence set forth in SEQ ID NO:16; and a VH region comprising the amino acid sequence set forth in SEQ ID NO:20.

In another aspect, the anti-C1s antibody comprises: a VL region comprising the amino acid sequence set forth in SEQ ID NO:16; and a VH region comprising the amino acid sequence set forth in SEQ ID NO:21.

In another aspect, the anti-C1s antibody comprises: a VL region comprising the amino acid sequence set forth in SEQ ID NO:17; and a VH region comprising the amino acid sequence set forth in SEQ ID NO:18.

In another aspect, the anti-C1s antibody comprises: a VL region comprising the amino acid sequence set forth in SEQ ID NO:17; and a VH region comprising the amino acid sequence set forth in SEQ ID NO:19.

In another aspect, the anti-C1s antibody comprises: a VL region comprising the amino acid sequence set forth in SEQ ID NO:17; and a VH region comprising the amino acid sequence set forth in SEQ ID NO:20.

In another aspect, the anti-C1s antibody comprises: a VL region comprising the amino acid sequence set forth in SEQ ID NO:17; and a VH region comprising the amino acid sequence set forth in SEQ ID NO:21.

In some aspects of the disclosure, the anti-C1s antibody comprises a heavy chain constant region of the isotype IgG1, IgG2, IgG3, or IgG4. In some aspects, the anti-C1s antibody is selected from the group consisting of a Fab fragment, a F(ab′)₂ fragment, a scFv, and a Fv.

In some aspects, the administration of the anti-C1s antibody is via subcutaneous administration, intravenous administration, or intramuscular administration.

In some aspects, the method of treating a complement-mediated disease or disorder in an individual comprises: a) administering a first dose of the anti-C1s antibody at day 1; b) administering a second dose of the anti-C1s antibody at day 8; and c) administering the anti-C1s antibody every other week following the day 8 dose.

The present disclosure also provides for a method of inhibiting activation of complement component C4 in an individual in need thereof, the method comprising administering an anti-C1s antibody to the individual, where the anti-C1s antibody is administered in an amount of 5.5 g. In other embodiments, the present disclosure provides for a method of inhibiting activation of complement component C4 in an individual in need thereof, the method comprising administering an anti-C1s antibody to the individual, where the anti-C1s antibody is administered in an amount of 6.5 g if the individual weighs less than about 75 kg. In some embodiments, the present disclosure provides for a method of inhibiting activation of complement component C4 in an individual in need thereof, the method comprising administering an anti-C1s antibody to the individual, where the anti-C1s antibody is administered in an amount of 7.5 g if the individual weighs about 75 kg or more.

In some aspects, the anti-C1s antibody is administered to the individual every other week.

In some aspects, the anti-C1s antibody comprises light chain complementarity determining regions (CDRs) of an antibody light chain variable region comprising amino acid sequence SEQ ID NO:7 and heavy chain CDRs of an antibody heavy chain variable region comprising amino acid sequence SEQ ID NO:8.

In some aspects, the anti-C1s antibody is humanized. In some aspects, the humanized antibody comprises a humanized light chain framework region and/or a humanized heavy chain framework region.

In some aspects, the anti-C1s antibody comprises: a) i) a light chain variable region comprising a complementarity-determining region (CDR) comprising a CDR-L1 having the amino acid sequence of SEQ ID NO:1, a CDR-L2 having the amino acid sequence of SEQ ID NO:2, a CDR-L3 having the amino acid sequence of SEQ ID NO:3; and ii) a heavy chain variable region comprising a CDR comprising a CDR-H1 having amino acid sequence SEQ ID NO:4, a CDR-H2 having amino acid sequence SEQ ID NO:5, and a CDR-H3 having amino acid sequence SEQ ID NO:6.

In some aspects, the anti-C1s antibody comprises: i) a light chain variable region comprising a complementarity-determining region (CDR) comprising a CDR-L1 having the amino acid sequence of SEQ ID NO:10, a CDR-L2 having the amino acid sequence of SEQ ID NO:11, a CDR-L3 having the amino acid sequence of SEQ ID NO:3; and ii) a heavy chain variable region comprising a CDR comprising a CDR-H1 having amino acid sequence SEQ ID NO:12, a CDR-H2 having amino acid sequence SEQ ID NO:13, and a CDR-H3 having amino acid sequence SEQ ID NO:14.

In another aspect, the anti-C1s antibody comprises: a VL region comprising the amino acid sequence set forth in SEQ ID NO:15; and a VH region comprising the amino acid sequence set forth in SEQ ID NO:18.

In another aspect, the anti-C1s antibody comprises: a VL region comprising the amino acid sequence set forth in SEQ ID NO:15; and a VH region comprising the amino acid sequence set forth in SEQ ID NO:19.

In another aspect, the anti-C1s antibody comprises: a VL region comprising the amino acid sequence set forth in SEQ ID NO:15; and a VH region comprising the amino acid sequence set forth in SEQ ID NO:20.

In another aspect, the anti-C1s antibody comprises: a VL region comprising the amino acid sequence set forth in SEQ ID NO:15; and a VH region comprising the amino acid sequence set forth in SEQ ID NO:21.

In another aspect, the anti-C1s antibody comprises: a VL region comprising the amino acid sequence set forth in SEQ ID NO:16; and a VH region comprising the amino acid sequence set forth in SEQ ID NO:18.

In another aspect, the anti-C1s antibody comprises: a VL region comprising the amino acid sequence set forth in SEQ ID NO:16; and a VH region comprising the amino acid sequence set forth in SEQ ID NO:19.

In another aspect, the anti-C1s antibody comprises: a VL region comprising the amino acid sequence set forth in SEQ ID NO:16; and a VH region comprising the amino acid sequence set forth in SEQ ID NO:20.

In another aspect, the anti-C1s antibody comprises: a VL region comprising the amino acid sequence set forth in SEQ ID NO:16; and a VH region comprising the amino acid sequence set forth in SEQ ID NO:21.

In another aspect, the anti-C1s antibody comprises: a VL region comprising the amino acid sequence set forth in SEQ ID NO:17; and a VH region comprising the amino acid sequence set forth in SEQ ID NO:18.

In another aspect, the anti-C1s antibody comprises: a VL region comprising the amino acid sequence set forth in SEQ ID NO:17; and a VH region comprising the amino acid sequence set forth in SEQ ID NO:19.

In another aspect, the anti-C1s antibody comprises: a VL region comprising the amino acid sequence set forth in SEQ ID NO:17; and a VH region comprising the amino acid sequence set forth in SEQ ID NO:20.

In another aspect, the anti-C1s antibody comprises: a VL region comprising the amino acid sequence set forth in SEQ ID NO:17; and a VH region comprising the amino acid sequence set forth in SEQ ID NO:21.

In some aspects of the disclosure, the anti-C1s antibody comprises a heavy chain constant region of the isotype IgG1, IgG2, IgG3, or IgG4. In some aspects of the disclosure, the anti-C1s antibody is selected from the group consisting of a Fab fragment, a F(ab′)₂ fragment, a scFv, and a Fv.

In some aspects, the administration of the anti-C1s antibody is via subcutaneous administration, intravenous administration, or intramuscular administration.

In some aspects, the method of inhibiting activation of complement component C4 in an individual in need thereof comprises: a) administering a first dose of the anti-C1s antibody at day 1; b) administering a second dose of the anti-C1s antibody at day 8; and c) administering the anti-C1s antibody every other week following the day 8 dose.

The present disclosure also provides a method of treating a complement-mediated disease or disorder in a subject in need thereof, the method comprising administering an effective dose of an anti-C1s antibody to the subject, wherein the serum concentration of the anti-C1s antibody after the administration is at least about 20 μg/mL, at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, or at least about 100 μg/mL.

In some aspects, the serum concentration of the anti-C1s antibody after the administration is between about 20 μg/mL and about 100 μg/mL, about 20 μg/mL and about 90 μg/mL, about 20 μg/mL and about 80 μg/mL, about 20 μg/mL and about 70 μg/mL, about 20 μg/mL and about 60 μg/mL, about 20 μg/mL and about 50 μg/mL, about 20 μg/mL and about 40 μg/mL, or about 20 μg/mL and about 30 μg/mL.

In some aspects, the serum concentration of the anti-C1s antibody is measured by a direct binding Enzyme-Linked Immunosorbent Assay (ELISA).

In some aspects, the effective dose of the anti-C1s antibody is at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg, at least about 105 mg/kg, at least about 110 mg/kg, at least about 115 mg/kg, at least about 120 mg/kg, at least about 125 mg/kg, at least about 130 mg/kg, at least about 135 mg/kg, at least about 140 mg/kg, at least about 145 mg/kg, at least about 150 mg/kg, at least about 155 mg/kg, at least about 160 mg/kg, at least about 165 mg/kg, at least about 170 mg/kg, at least about 175 mg/kg, at least about 180 mg/kg, at least about 185 mg/kg, at least about 190 mg/kg, at least about 195 mg/kg, or at least about 200 mg/kg. In other aspects, the effective dose of the anti-C1s antibody is about 4 g to about 10 g.

In some aspects, the effective dose is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In other aspects, the effective dose is between about 4 g and about 10 g, about 5 g and about 8 g, about 5.5 g and about 7.5 g, about 6.5 g and about 7.5 g, or about 6.5 g and about 8.5 g. In some aspects, the effective dose is between about 4 g and about 9 g, between about 5 g and about 8 g, between about 5.5 g and about 7.5 g, between about 6 g and about 8 g, or between about 6.5 g and about 7.5 g.

In some aspects, the effective dose is about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg. In some aspects, the effective dose is about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

In some aspects, the anti-C1s antibody is administered at a dosing interval of five days, six days, seven days, eight days, nine days, ten days, eleven days, twelve days, thirteen days, fourteen days, fifteen days, sixteen days, seventeen days, eighteen days, nineteen days, twenty days, twenty one days, twenty two days, twenty three days, twenty four days, twenty five days, twenty six days, twenty seven days, twenty eight days, twenty nine days, thirty days, or thirty one days.

In some aspects, the anti-C1s antibody is administered at a dosing interval of a week, two weeks, three weeks, four weeks, or a month.

In some aspects, the anti-C1s antibody increases the number of reticulocytes in the subject's blood after the administration.

The present disclosure also provides a method of increasing the number of reticulocytes in the blood of a subject in need thereof, comprising administering to the subject an effective dose of an anti-C1s antibody.

In some aspects, the anti-C1s antibody increases the number of reticulocytes in the blood of the subject after the administration at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2.0 fold, at least about 2.1 fold, at least about 2.2 fold, at least about 2.3 fold, at least about 2.4 fold, at least about 2.5 fold, at least about 2.6 fold, at least about 2.7 fold, at least about 2.8 fold, at least about 2.9 fold, at least about 3.0 fold, at least about 4 fold, at least about 5 fold, at least about 6 fold, at least about 7 fold, at least about 8 fold, at least about 9 fold, or at least about 10 fold. In some aspects, the anti-C1s antibody increases the number of reticulocytes in the blood of the subject within about 24 hours of the administration.

In some aspects of the present disclosure, the anti-C1s antibody increases the level of hemoglobin in the subject. In some aspects, the anti-C1s antibody increases the level of hemoglobin in the subject at least about 1.0 g/dL, 1.1 g/dL, 1.2 g/dL, 1.3 g/dL, 1.4 g/dL, 1.5 g/dL, 1.6 g/dL, 1.7 g/dL, 1.8 g/dL, 1.9 g/dL, 2.0 g/dL, 2.1 g/dL, 2.2 g/dL, 2.3 g/dL, 2.4 g/dL, 2.5 g/dL, 2.6 g/dL, 2.7 g/dL, 2.8 g/dL, 2.9 g/dL, 3.0 g/dL, 3.1 g/dL, 3.2 g/dL, 3.3 g/dL, 3.4 g/dL, 3.5 g/dL, 3.6 g/dL, 3.7 g/dL, 3.8 g/dL, 3.9 g/dL, 4.0 g/dL, 4.1 g/dL, 4.2 g/dL, 4.3 g/dL, 4.4 g/dL, 4.5 g/dL, 4.6 g/dL, 4.7 g/dL, 4.8 g/dL, 4.9 g/dL, 5.0 g/dL, 5.1 g/dL, 5.2 g/dL, 5.3 g/dL, 5.4 g/dL, 5.5 g/dL, 5.6 g/dL, 5.7 g/dL, 5.8 g/dL, 5.9 g/dL, or 6.0 g/dL. In some aspects, the level of hemoglobin in the subject is increased at least by 1.6 g/dL within seven days from the administration. In some aspects, the level of hemoglobin in the subject is increased up to 3.9 g/dL within six weeks from the administration.

In some aspects of the present disclosure, the anti-C1s antibody decreases the percentage of C3d positive erythrocytes in the blood of the subject. In some aspects, the percentage of C3d positive erythrocytes in the blood of the subject is decreased at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the percentage of C3d positive erythrocytes in the blood of the subject prior to the administration. In other aspects, the percentage of C3d positive erythrocytes in the blood of the subject is decreased to about 0%, about 1%, about 2%, about 3%, about 4%, or about 5%.

In some aspects, the anti-C1s antibody decreases the level of bilirubin in the subject. In some aspects, the level of bilirubin in the subject is decreased to be lower than about 2.5 mg/dL, 2.4 mg/dL, 2.3 mg/dL, 2.2 mg/dL, 2.1 mg/dL, 2.0 mg/dL, 1.9 mg/dL, 1.8 mg/dL, 1.7 mg/dL, 1.6 mg/dL, 1.5 mg/dL, 1.4 mg/dL, 1.3 mg/dL, 1.2 mg/dL, 1.1 mg/dL, 1.0 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL.

In some aspects of the present disclosure, the anti-C1s antibody cross-competes with an antibody comprising: a) a VL region comprising the amino acid sequence set forth in SEQ ID NO:15; and a VH region comprising the amino acid sequence set forth in SEQ ID NO:18; b) a VL region comprising the amino acid sequence set forth in SEQ ID NO:15; and a VH region comprising the amino acid sequence set forth in SEQ ID NO:19; c) a VL region comprising the amino acid sequence set forth in SEQ ID NO:15; and a VH region comprising the amino acid sequence set forth in SEQ ID NO:20; d) a VL region comprising the amino acid sequence set forth in SEQ ID NO:15; and a VH region comprising the amino acid sequence set forth in SEQ ID NO:21; e) a VL region comprising the amino acid sequence set forth in SEQ ID NO:16; and a VH region comprising the amino acid sequence set forth in SEQ ID NO:18; f) a VL region comprising the amino acid sequence set forth in SEQ ID NO:16; and a VH region comprising the amino acid sequence set forth in SEQ ID NO:19; g) a VL region comprising the amino acid sequence set forth in SEQ ID NO:16; and a VH region comprising the amino acid sequence set forth in SEQ ID NO:20; h) a VL region comprising the amino acid sequence set forth in SEQ ID NO:16; and a VH region comprising the amino acid sequence set forth in SEQ ID NO:21; i) a VL region comprising the amino acid sequence set forth in SEQ ID NO:17; and a VH region comprising the amino acid sequence set forth in SEQ ID NO:18; j) a VL region comprising the amino acid sequence set forth in SEQ ID NO:17; and a VH region comprising the amino acid sequence set forth in SEQ ID NO:19; k) a VL region comprising the amino acid sequence set forth in SEQ ID NO:17; and a VH region comprising the amino acid sequence set forth in SEQ ID NO:20; or 1) a VL region comprising the amino acid sequence set forth in SEQ ID NO:17; and a VH region comprising the amino acid sequence set forth in SEQ ID NO:21.

In some aspects of the present disclosure, the anti-C1s antibody binds to the same epitope as an antibody comprising: a) a VL region comprising the amino acid sequence set forth in SEQ ID NO:15; and a VH region comprising the amino acid sequence set forth in SEQ ID NO:18; b) a VL region comprising the amino acid sequence set forth in SEQ ID NO:15; and a VH region comprising the amino acid sequence set forth in SEQ ID NO:19; c) a VL region comprising the amino acid sequence set forth in SEQ ID NO:15; and a VH region comprising the amino acid sequence set forth in SEQ ID NO:20; d) a VL region comprising the amino acid sequence set forth in SEQ ID NO:15; and a VH region comprising the amino acid sequence set forth in SEQ ID NO:21; e) a VL region comprising the amino acid sequence set forth in SEQ ID NO:16; and a VH region comprising the amino acid sequence set forth in SEQ ID NO:18; f) a VL region comprising the amino acid sequence set forth in SEQ ID NO:16; and a VH region comprising the amino acid sequence set forth in SEQ ID NO:19; g) a VL region comprising the amino acid sequence set forth in SEQ ID NO:16; and a VH region comprising the amino acid sequence set forth in SEQ ID NO:20; h) a VL region comprising the amino acid sequence set forth in SEQ ID NO:16; and a VH region comprising the amino acid sequence set forth in SEQ ID NO:21; i) a VL region comprising the amino acid sequence set forth in SEQ ID NO:17; and a VH region comprising the amino acid sequence set forth in SEQ ID NO:18; j) a VL region comprising the amino acid sequence set forth in SEQ ID NO:17; and a VH region comprising the amino acid sequence set forth in SEQ ID NO:19; k) a VL region comprising the amino acid sequence set forth in SEQ ID NO:17; and a VH region comprising the amino acid sequence set forth in SEQ ID NO:20; or 1) a VL region comprising the amino acid sequence set forth in SEQ ID NO:17; and a VH region comprising the amino acid sequence set forth in SEQ ID NO:21.

In some aspects of the present disclosure, the anti-C1s antibody comprises: a) i) a light chain variable region and a heavy chain variable region, wherein the light chain variable region (VL) comprises CDR-L1 having the amino acid sequence of SEQ ID NO:1, CDR-L2 having the amino acid sequence of SEQ ID NO:2, CDR-L3 having the amino acid sequence of SEQ ID NO:3; and ii) a heavy chain variable region (VH) comprising CDR-H1 having amino acid sequence SEQ ID NO:4, CDR-H2 having amino acid sequence SEQ ID NO:5, and CDR-H3 having amino acid sequence SEQ ID NO:6; or b) i) a light chain variable region comprising CDR-L1 having the amino acid sequence of SEQ ID NO:10, CDR-L2 having the amino acid sequence of SEQ ID NO:11, CDR-L3 having the amino acid sequence of SEQ ID NO:3; and ii) a heavy chain variable region comprising CDR-H1 having amino acid sequence SEQ ID NO:12, CDR-H2 having amino acid sequence SEQ ID NO:13, and CDR-H3 having amino acid sequence SEQ ID NO:14.

In some aspects of the disclosure, the anti-C1s antibody comprises: a) a VL region comprising the amino acid sequence set forth in SEQ ID NO:15; and a VH region comprising the amino acid sequence set forth in SEQ ID NO:18; b) a VL region comprising the amino acid sequence set forth in SEQ ID NO:15; and a VH region comprising the amino acid sequence set forth in SEQ ID NO:19; c) a VL region comprising the amino acid sequence set forth in SEQ ID NO:15; and a VH region comprising the amino acid sequence set forth in SEQ ID NO:20; d) a VL region comprising the amino acid sequence set forth in SEQ ID NO:15; and a VH region comprising the amino acid sequence set forth in SEQ ID NO:21; e) a VL region comprising the amino acid sequence set forth in SEQ ID NO:16; and a VH region comprising the amino acid sequence set forth in SEQ ID NO:18; f) a VL region comprising the amino acid sequence set forth in SEQ ID NO:16; and a VH region comprising the amino acid sequence set forth in SEQ ID NO:19; g) a VL region comprising the amino acid sequence set forth in SEQ ID NO:16; and a VH region comprising the amino acid sequence set forth in SEQ ID NO:20; h) a VL region comprising the amino acid sequence set forth in SEQ ID NO:16; and a VH region comprising the amino acid sequence set forth in SEQ ID NO:21; i) a VL region comprising the amino acid sequence set forth in SEQ ID NO:17; and a VH region comprising the amino acid sequence set forth in SEQ ID NO:18; j) a VL region comprising the amino acid sequence set forth in SEQ ID NO:17; and a VH region comprising the amino acid sequence set forth in SEQ ID NO:19; k) a VL region comprising the amino acid sequence set forth in SEQ ID NO:17; and a VH region comprising the amino acid sequence set forth in SEQ ID NO:20; or 1) a VL region comprising the amino acid sequence set forth in SEQ ID NO:17; and a VH region comprising the amino acid sequence set forth in SEQ ID NO:21.

In some aspects, the anti-C1s antibody comprises a heavy chain constant region of the isotype IgG1, IgG2, IgG3, or IgG4.

In some aspects, the anti-C1s antibody is selected from the group consisting of a Fab fragment, a F(ab′)2 fragment, a scFv, and a Fv.

In some aspects, the administration is via subcutaneous administration, intravenous administration, or intramuscular administration.

### EMBODIMENTS

E1. A method of treating a complement-mediated disease or disorder in an individual, the method comprising administering an anti-C1s antibody to the individual, where the anti-C1s antibody is administered in an amount of 5.5 g.

E2. The method of E1, wherein the anti-C1s antibody is administered to the individual every other week.

E3. The method of E1 or E2, wherein the anti-C1s antibody comprises light chain complementarity determining regions (CDRs) of an antibody light chain variable region comprising amino acid sequence SEQ ID NO:7 and heavy chain CDRs of an antibody heavy chain variable region comprising amino acid sequence SEQ ID NO:8.

E4. The method of any one of E1-E3, wherein the anti-C1s antibody is humanized.

E5. The method of E4, wherein the humanized antibody comprises a humanized light chain framework region and/or a humanized heavy chain framework region.

E6. The method of any one of E1-E5, wherein the anti-C1s antibody comprises:
a) i) a light chain variable region comprising a complementarity-determining region (CDR) comprising a CDR-L1 having the amino acid sequence of SEQ ID NO:1, a CDR-L2 having the amino acid sequence of SEQ ID NO:2, a CDR-L3 having the amino acid sequence of SEQ ID NO:3; and ii) a heavy chain variable region comprising a CDR comprising a CDR-H1 having amino acid sequence SEQ ID NO:4, a CDR-H2 having amino acid sequence SEQ ID NO:5, and a CDR-H3 having amino acid sequence SEQ ID NO:6;
b) i) a light chain variable region comprising a complementarity-determining region (CDR) comprising a CDR-L1 having the amino acid sequence of SEQ ID NO:10, a CDR-L2 having the amino acid sequence of SEQ ID NO:11, a CDR-L3 having the amino acid sequence of SEQ ID NO:3; and ii) a heavy chain variable region comprising a CDR comprising a CDR-H1 having amino acid sequence SEQ ID NO:12, a CDR-H2 having amino acid sequence SEQ ID NO:13, and a CDR-H3 having amino acid sequence SEQ ID NO:14;
c) a VL region comprising the amino acid sequence set forth in SEQ ID NO:15; and a VH region comprising the amino acid sequence set forth in SEQ ID NO:18;
d) a VL region comprising the amino acid sequence set forth in SEQ ID NO:15; and a VH region comprising the amino acid sequence set forth in SEQ ID NO:19;
e) a VL region comprising the amino acid sequence set forth in SEQ ID NO:15; and a VH region comprising the amino acid sequence set forth in SEQ ID NO:20;
f) a VL region comprising the amino acid sequence set forth in SEQ ID NO:15; and a VH region comprising the amino acid sequence set forth in SEQ ID NO:21;
g) a VL region comprising the amino acid sequence set forth in SEQ ID NO:16; and a VH region comprising the amino acid sequence set forth in SEQ ID NO:18;
h) a VL region comprising the amino acid sequence set forth in SEQ ID NO:16; and a VH region comprising the amino acid sequence set forth in SEQ ID NO:19;
i) a VL region comprising the amino acid sequence set forth in SEQ ID NO:16; and a VH region comprising the amino acid sequence set forth in SEQ ID NO:20;
j) a VL region comprising the amino acid sequence set forth in SEQ ID NO:16; and a VH region comprising the amino acid sequence set forth in SEQ ID NO:21;
k) a VL region comprising the amino acid sequence set forth in SEQ ID NO:17; and a VH region comprising the amino acid sequence set forth in SEQ ID NO:18;
l) a VL region comprising the amino acid sequence set forth in SEQ ID NO:17; and a VH region comprising the amino acid sequence set forth in SEQ ID NO:19;
m) a VL region comprising the amino acid sequence set forth in SEQ ID NO:17; and a VH region comprising the amino acid sequence set forth in SEQ ID NO:20; or
n) a VL region comprising the amino acid sequence set forth in SEQ ID NO:17; and a VH region comprising the amino acid sequence set forth in SEQ ID NO:21.

E7. The method of any one of E1-E6, wherein the anti-C1s antibody comprises a heavy chain constant region of the isotype IgG1, IgG2, IgG3, or IgG4.

E8. The method of any one of E1-E6, wherein the anti-C1s antibody is selected from the group consisting of a Fab fragment, a F(ab′)2 fragment, a scFv, and a Fv.

E9. The method of any one of E1-E8, wherein said administering is via subcutaneous administration, intravenous administration, or intramuscular administration.

E10. The method of any one of E1-E9, comprising:
a) administering a first dose of the anti-C1s antibody at day 1;
b) administering a second dose of the anti-C1s antibody at day 8; and
c) administering the anti-C1s antibody every other week following the day 8 dose.

E11. A method of inhibiting activation of complement component C4 in an individual in need thereof, the method comprising administering an anti-C1s antibody to the individual, where the anti-C1s antibody is administered in an amount of 5.5 g.

E12. The method of E1, wherein the anti-C1s antibody is administered to the individual every other week.

E13. The method of E11 or E12, wherein the anti-C1s antibody comprises light chain complementarity determining regions (CDRs) of an antibody light chain variable region comprising amino acid sequence SEQ ID NO:7 and heavy chain CDRs of an antibody heavy chain variable region comprising amino acid sequence SEQ ID NO:8.

E14. The method of any one of E1-E13, wherein the anti-C1s antibody is humanized.

E15. The method of E14, wherein the humanized antibody comprises a humanized light chain framework region and/or a humanized heavy chain framework region.

E16. The method of any one of E1-E15, wherein the anti-C1s antibody comprises:
a) i) a light chain variable region comprising a complementarity-determining region (CDR) comprising a CDR-Li having the amino acid sequence of SEQ ID NO:1, a CDR-L2 having the amino acid sequence of SEQ ID NO:2, a CDR-L3 having the amino acid sequence of SEQ ID NO:3; and ii) a heavy chain variable region comprising a CDR comprising a CDR-H1 having amino acid sequence SEQ ID NO:4, a CDR-H2 having amino acid sequence SEQ ID NO:5, and a CDR-H3 having amino acid sequence SEQ ID NO:6;
b) i) a light chain variable region comprising a complementarity-determining region (CDR) comprising a CDR-L1 having the amino acid sequence of SEQ ID NO:10, a CDR-L2 having the amino acid sequence of SEQ ID NO:11, a CDR-L3 having the amino acid sequence of SEQ ID NO:3; and ii) a heavy chain variable region comprising a CDR comprising a CDR-H1 having amino acid sequence SEQ ID NO:12, a CDR-H2 having amino acid sequence SEQ ID NO:13, and a CDR-H3 having amino acid sequence SEQ ID NO:14;
c) a VL region comprising the amino acid sequence set forth in SEQ ID NO:15; and a VH region comprising the amino acid sequence set forth in SEQ ID NO:18;
d) a VL region comprising the amino acid sequence set forth in SEQ ID NO:15; and a VH region comprising the amino acid sequence set forth in SEQ ID NO:19;
e) a VL region comprising the amino acid sequence set forth in SEQ ID NO:15; and a VH region comprising the amino acid sequence set forth in SEQ ID NO:20;
f) a VL region comprising the amino acid sequence set forth in SEQ ID NO:15; and a VH region comprising the amino acid sequence set forth in SEQ ID NO:21;
g) a VL region comprising the amino acid sequence set forth in SEQ ID NO:16; and a VH region comprising the amino acid sequence set forth in SEQ ID NO:18;
h) a VL region comprising the amino acid sequence set forth in SEQ ID NO:16; and a VH region comprising the amino acid sequence set forth in SEQ ID NO:19;
i) a VL region comprising the amino acid sequence set forth in SEQ ID NO:16; and a VH region comprising the amino acid sequence set forth in SEQ ID NO:20;
j) a VL region comprising the amino acid sequence set forth in SEQ ID NO:16; and a VH region comprising the amino acid sequence set forth in SEQ ID NO:21;
k) a VL region comprising the amino acid sequence set forth in SEQ ID NO:17; and a VH region comprising the amino acid sequence set forth in SEQ ID NO:18;
l) a VL region comprising the amino acid sequence set forth in SEQ ID NO:17; and a VH region comprising the amino acid sequence set forth in SEQ ID NO:19;
m) a VL region comprising the amino acid sequence set forth in SEQ ID NO:17; and a VH region comprising the amino acid sequence set forth in SEQ ID NO:20; or
n) a VL region comprising the amino acid sequence set forth in SEQ ID NO:17; and a VH region comprising the amino acid sequence set forth in SEQ ID NO:21.

E17. The method of any one of E11-E16, wherein the anti-C1s antibody comprises a heavy chain constant region of the isotype IgG1, IgG2, IgG3, or IgG4.

E18. The method of any one of E11-E16, wherein the anti-C1s antibody is selected from the group consisting of a Fab fragment, a F(ab′)2 fragment, a scFv, and a Fv.

E19. The method of any one of E11-E18, wherein said administering is via subcutaneous administration, intravenous administration, or intramuscular administration.

E20. The method of any one of E11-E19, comprising:
a) administering a first dose of the anti-C1s antibody at day 1;
b) administering a second dose of the anti-C1s antibody at day 8; and
c) administering the anti-C1s antibody every other week following the day 8 dose.

E21. A method of treating a complement-mediated disease or disorder in a subject in need thereof, the method comprising administering an effective dose of an anti-C1s antibody to the subject, where the serum concentration of the anti-C1s antibody after the administering is at least about 20 μg/mL, at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, or at least about 100 μg/mL.

E22. The method of E21, wherein the serum concentration of the anti-C1s antibody after the administering is between about 20 μg/mL and about 100 μg/mL, about 20 μg/mL and about 90 μg/mL, about 20 μg/mL and about 80 μg/mL, about 20 μg/mL and about 70 μg/mL, about 20 μg/mL and about 70 μg/mL, about 20 μg/mL and about 60 μg/mL, about 20 μg/mL and about 50 μg/mL, about 20 μg/mL and about 40 μg/mL, or about 20 μg/mL and about 30 μg/mL.

E23. The method of E21 or E22, wherein the serum concentration of the anti-C1s antibody is measured by a direct binding Enzyme-Linked Immunosorbent Assay (ELISA).

E24. The method of any one of E21 to E23, wherein the effective dose is at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg, at least about 105 mg/kg, at least about 110 mg/kg, at least about 115 mg/kg, at least about 120 mg/kg, at least about 125 mg/kg, at least about 130 mg/kg, at least about 135 mg/kg, at least about 140 mg/kg, at least about 145 mg/kg, at least about 150 mg/kg, at least about 155 mg/kg, at least about 160 mg/kg, at least about 165 mg/kg, at least about 170 mg/kg, at least about 175 mg/kg, at least about 180 mg/kg, at least about 185 mg/kg, at least about 190 mg/kg, at least about 195 mg/kg, or at least about 200 mg/kg or about 4 g to 10 g.

E25. The method of any one of E21 to E23, wherein the effective dose is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg or about 4 g and about 10 g, about 5 g and about 8 g, about 5.5 g and about 7.5 g, about 6.5 g and about 7.5 g, or about 6.5 g and about 8.5 g.

E26. The method of E25, wherein the effective dose is about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg or 4 g, 4.5 g, 5 g, 5.5 g, 6 g, 6.5 g, 7 g, 7.5 g, 8 g, 8.5 g, 9 g, 9.5 g, or 10 g.

E27. The method of any one of E21 to E26, wherein the anti-C1s antibody is administered at a dosing interval of five days, six days, seven days, eight days, nine days, ten days, eleven days, twelve days, thirteen days, fourteen days, fifteen days, sixteen days, seventeen days, eighteen days, nineteen days, twenty days, twenty one days, twenty two days, twenty three days, twenty four days, twenty five days, twenty six days, twenty seven days, twenty eight days, twenty nine days, thirty days, or thirty one days.

E28. The method of any one of E21 to E26, wherein the anti-C1s antibody is administered at a dosing interval of a week, two weeks, three weeks, four weeks, or a month.

E29. The method of any one of E21 to E28, wherein the anti-C1s antibody increases the number of reticulocytes in the subject's blood after the administering.

E30. A method of increasing the number of reticulocytes in the blood of a subject in need thereof, comprising administering to the subject an effective dose of an anti-C1s antibody.

E31. The method of E29 or E30, wherein the anti-C1s antibody increases the number of reticulocytes in the blood of the subject after the administering at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 4 fold, at least 5 fold, at least 6 fold, at least 7 fold, at least 8 fold, at least 9 fold, or at least 10 fold.

E32. The method of any one of E29 to E31, wherein the anti-C1s antibody increases the number of reticulocytes in the blood of the subject within about 24 hours of the administering.

E33. The method of any one of E1 to E32, wherein the anti-C1s antibody increases the level of hemoglobin in the subject.

E34. The method of E33, wherein the anti-C1s antibody increases the level of hemoglobin in the subject at least about 1.0 g/dL, 1.1 g/dL, 1.2 g/dL, 1.3 g/dL, 1.4 g/dL, 1.5 g/dL, 1.6 g/dL, 1.7 g/dL, 1.8 g/dL, 1.9 g/dL, 2.0 g/dL, 2.1 g/dL, 2.2 g/dL, 2.3 g/dL, 2.4 g/dL, 2.5 g/dL, 2.6 g/dL, 2.7 g/dL, 2.8 g/dL, 2.9 g/dL, 3.0 g/dL, 3.1 g/dL, 3.2 g/dL, 3.3 g/dL, 3.4 g/dL, 3.5 g/dL, 3.6 g/dL, 3.7 g/dL, 3.8 g/dL, 3.9 g/dL, 4.0 g/dL, 4.1 g/dL, 4.2 g/dL, 4.3 g/dL, 4.4 g/dL, 4.5 g/dL, 4.6 g/dL, 4.7 g/dL, 4.8 g/dL, 4.9 g/dL, 5.0 g/dL, 5./1 g/dL, 5.2 g/dL, 5.3 g/dL, 5.4 g/dL, 5.5 g/dL, 5.6 g/dL, 5.7 g/dL, 5.8 g/dL, 5.9 g/dL, or 6.0 g/dL.

E35. The method of E33, wherein the level of hemoglobin in the subject is increased at least by 1.6 g/dL within seven days from the administering.

E36. The method of E33, wherein the level of hemoglobin in the subject is increased up to 3.9 g/dL within six weeks from the administering.

E37. The method of any one of E1 to E36, wherein the anti-C1s antibody decreases the percentage of C3d positive erythrocytes in the subject, e.g., blood.

E38. The method of E37, wherein the percentage of C3d positive erythrocytes in the subject is decreased at least 5%, at least 10%, at least 15%, at least 20%, at least 25%, at least 30%, at least 35%, at least 40%, at least 45%, at least 50%, at least about 55%, at least about 60%, at least about 65%, at least about 70%, at least about 75%, at least about 80%, at least about 85%, at least about 90%, at least about 95%, or about 100% compared to the percentage of C3d positive erythrocytes in the subject prior to the administering.

E39. The method of any one of E1 to E38, wherein the anti-C1s antibody decreases the level of bilirubin in the subject, e.g., blood.

E40. The method of E39, wherein the level of bilirubin in the subject is decreased to be lower than 2.5 mg/dL, 2.4 mg/dL, 2.3 mg/dL, 2.2 mg/dL, 2.1 mg/dL, 2.0 mg/dL, 1.9 mg/dL, 1.8 mg/dL, 1.7 mg/dL, 1.6 mg/dL, 1.5 mg/dL, 1.4 mg/dL, 1.3 mg/dL, 1.2 mg/dL, 1.1 mg/dL, 1.0 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL.

E41. The method of any one of E21 to E40, wherein the anti-C1s antibody cross-competes with an antibody comprising:
a) a VL region comprising the amino acid sequence set forth in SEQ ID NO:15; and a VH region comprising the amino acid sequence set forth in SEQ ID NO:18;
b) a VL region comprising the amino acid sequence set forth in SEQ ID NO:15; and a VH region comprising the amino acid sequence set forth in SEQ ID NO:19;
c) a VL region comprising the amino acid sequence set forth in SEQ ID NO:15; and a VH region comprising the amino acid sequence set forth in SEQ ID NO:20;
d) a VL region comprising the amino acid sequence set forth in SEQ ID NO:15; and a VH region comprising the amino acid sequence set forth in SEQ ID NO:21;
e) a VL region comprising the amino acid sequence set forth in SEQ ID NO:16; and a VH region comprising the amino acid sequence set forth in SEQ ID NO:18;
f) a VL region comprising the amino acid sequence set forth in SEQ ID NO:16; and a VH region comprising the amino acid sequence set forth in SEQ ID NO:19;
g) a VL region comprising the amino acid sequence set forth in SEQ ID NO:16; and a VH region comprising the amino acid sequence set forth in SEQ ID NO:20;
h) a VL region comprising the amino acid sequence set forth in SEQ ID NO:16; and a VH region comprising the amino acid sequence set forth in SEQ ID NO:21;
i) a VL region comprising the amino acid sequence set forth in SEQ ID NO:17; and a VH region comprising the amino acid sequence set forth in SEQ ID NO:18;
j) a VL region comprising the amino acid sequence set forth in SEQ ID NO:17; and a VH region comprising the amino acid sequence set forth in SEQ ID NO:19;
k) a VL region comprising the amino acid sequence set forth in SEQ ID NO:17; and a VH region comprising the amino acid sequence set forth in SEQ ID NO:20; or
l) a VL region comprising the amino acid sequence set forth in SEQ ID NO:17; and a VH region comprising the amino acid sequence set forth in SEQ ID NO:21.

E42. The method of any one of E21 to E41, wherein the anti-C1s antibody binds to the same epitope as an antibody comprising:
a) a VL region comprising the amino acid sequence set forth in SEQ ID NO:15; and a VH region comprising the amino acid sequence set forth in SEQ ID NO:18;
b) a VL region comprising the amino acid sequence set forth in SEQ ID NO:15; and a VH region comprising the amino acid sequence set forth in SEQ ID NO:19;
c) a VL region comprising the amino acid sequence set forth in SEQ ID NO:15; and a VH region comprising the amino acid sequence set forth in SEQ ID NO:20;
d) a VL region comprising the amino acid sequence set forth in SEQ ID NO:15; and a VH region comprising the amino acid sequence set forth in SEQ ID NO:21;
e) a VL region comprising the amino acid sequence set forth in SEQ ID NO:16; and a VH region comprising the amino acid sequence set forth in SEQ ID NO:18;
f) a VL region comprising the amino acid sequence set forth in SEQ ID NO:16; and a VH region comprising the amino acid sequence set forth in SEQ ID NO:19;
g) a VL region comprising the amino acid sequence set forth in SEQ ID NO:16; and a VH region comprising the amino acid sequence set forth in SEQ ID NO:20;
h) a VL region comprising the amino acid sequence set forth in SEQ ID NO:16; and a VH region comprising the amino acid sequence set forth in SEQ ID NO:21;
i) a VL region comprising the amino acid sequence set forth in SEQ ID NO:17; and a VH region comprising the amino acid sequence set forth in SEQ ID NO:18;
j) a VL region comprising the amino acid sequence set forth in SEQ ID NO:17; and a VH region comprising the amino acid sequence set forth in SEQ ID NO:19;
k) a VL region comprising the amino acid sequence set forth in SEQ ID NO:17; and a VH region comprising the amino acid sequence set forth in SEQ ID NO:20; or
l) a VL region comprising the amino acid sequence set forth in SEQ ID NO:17; and a VH region comprising the amino acid sequence set forth in SEQ ID NO:21.

E43. The method of any one of E21 to E42, wherein the anti-C1s antibody comprises:
a) i) a light chain variable region and a heavy chain variable region, wherein the light chain variable region (VL) comprises CDR-L1 having the amino acid sequence of SEQ ID NO:1, CDR-L2 having the amino acid sequence of SEQ ID NO:2, CDR-L3 having the amino acid sequence of SEQ ID NO:3; and ii) a heavy chain variable region (VH) comprising CDR-H1 having amino acid sequence SEQ ID NO:4, CDR-H2 having amino acid sequence SEQ ID NO:5, and CDR-H3 having amino acid sequence SEQ ID NO:6; or
b) i) a light chain variable region comprising CDR-L1 having the amino acid sequence of SEQ ID NO:10, CDR-L2 having the amino acid sequence of SEQ ID NO:11, CDR-L3 having the amino acid sequence of SEQ ID NO:3; and ii) a heavy chain variable region comprising CDR-H1 having amino acid sequence SEQ ID NO:12, CDR-H2 having amino acid sequence SEQ ID NO:13, and CDR-H3 having amino acid sequence SEQ ID NO:14.

E44. The method of any one of E21 to E43, wherein the anti-C1s antibody comprises:
a) a VL region comprising the amino acid sequence set forth in SEQ ID NO:15; and a VH region comprising the amino acid sequence set forth in SEQ ID NO:18;
b) a VL region comprising the amino acid sequence set forth in SEQ ID NO:15; and a VH region comprising the amino acid sequence set forth in SEQ ID NO:19;
c) a VL region comprising the amino acid sequence set forth in SEQ ID NO:15; and a VH region comprising the amino acid sequence set forth in SEQ ID NO:20;
d) a VL region comprising the amino acid sequence set forth in SEQ ID NO:15; and a VH region comprising the amino acid sequence set forth in SEQ ID NO:21;
e) a VL region comprising the amino acid sequence set forth in SEQ ID NO:16; and a VH region comprising the amino acid sequence set forth in SEQ ID NO:18;
f) a VL region comprising the amino acid sequence set forth in SEQ ID NO:16; and a VH region comprising the amino acid sequence set forth in SEQ ID NO:19;
g) a VL region comprising the amino acid sequence set forth in SEQ ID NO:16; and a VH region comprising the amino acid sequence set forth in SEQ ID NO:20;
h) a VL region comprising the amino acid sequence set forth in SEQ ID NO:16; and a VH region comprising the amino acid sequence set forth in SEQ ID NO:21;
i) a VL region comprising the amino acid sequence set forth in SEQ ID NO:17; and a VH region comprising the amino acid sequence set forth in SEQ ID NO:18;
j) a VL region comprising the amino acid sequence set forth in SEQ ID NO:17; and a VH region comprising the amino acid sequence set forth in SEQ ID NO:19;
k) a VL region comprising the amino acid sequence set forth in SEQ ID NO:17; and a VH region comprising the amino acid sequence set forth in SEQ ID NO:20; or
l) a VL region comprising the amino acid sequence set forth in SEQ ID NO:17; and a VH region comprising the amino acid sequence set forth in SEQ ID NO:21.

E45. The method of any one of E21 to E44, wherein the anti-C1s antibody comprises a heavy chain constant region of the isotype IgG1, IgG2, IgG3, or IgG4.

E46. The method of any one of E21 to E45, wherein the anti-C1s antibody is selected from the group consisting of a Fab fragment, a F(ab′)2 fragment, a scFv, and a Fv.

E47. The method of any one of E21 to E46, wherein the administering is via subcutaneous administration, intravenous administration, or intramuscular administration.

### BRIEF DESCRIPTION OF THE DRAWINGS

FIG. 1 shows a table of cold agglutinin disease (CAD) patient characteristics for patients administered an anti-C1s antibody.

FIG. 2A-2C show CAD patient laboratory parameters before and during treatment with BIVV009. FIG. 2A shows patient baseline laboratory parameters before treatment with BIVV009. FIG. 2B shows patient minimum and maximum laboratory parameters during treatment with BIVV009. FIG. 2C shows the maximal changes in laboratory parameters during treatment with BIVV009.

FIG. 3A-3B shows pharmacokinetics and pharmacodynamics of an anti-C1s antibody, BIVV009. FIG. 3A shows concentration response analysis of BIVV009 levels and classical pathway activity in serum samples taken from the normal healthy volunteers (NHV). FIG. 3B shows the average pharmacokinetic profile of BIVV009 in patients with cold agglutinin disease (CAD) (n=10).

FIG. 4A-4C shows the hematological response to BIVV009 infusion. Data are medians and interquartile ranges for 10 patients. FIG. 4A shows the levels of C3d positive erythrocytes (%) following BIVV009 administration. FIG. 4B (solid squares) shows the levels of hemoglobin (g/dL) following BIVV009 administration. The open triangles in FIG. 4B represent the median hemoglobin levels in the subgroup of patients with primary cold agglutinin disease using Berentsen's definition. FIG. 4C shows the levels of bilirubin (mg/dL) following BIVV009 administration. Data are medians and interquartile ranges for 10 patients.

FIG. 5 shows a plot of circulating bilirubin levels vs. BIVV009 concentration. The dotted line on the x-axis represents 20 μg/mL BIVV009 in serum, and the dotted line on the y-axis represents 1.2 mg/dL (upper limit of normal).

FIG. 6 shows a comparison of historical hemoglobin values to BIVV009 response in a patient with CAD. PRBC, packed red blood cells.

FIG. 7A-7F show the biochemical response pattern in a patient with CAD upon repeat administration of BIVV009. Arrows indicate BIVV009 administrations. BIVV009 dose levels are also provided above the solid bars. FIG. 7A shows reticulocyte levels (×10⁹/L) over time (days) after repeat administration of BIVV009. FIG. 7B shows hemoglobin levels (g/dL) over time (days) after repeat administration of BIVV009. FIG. 7C shows haptoglobin levels (mg/dL) over time (days) after repeat administration of BIVV009. FIG. 7D shows lactate dehydrogenase (LDH) levels (U/L) over time (days) after repeat administration of BIVV009. FIG. 7E shows serum classical complement pathway activity (CH50) over time (days) after repeat administration of BIVV009. FIG. 7F shows bilirubin levels (mg/dL) over time (days) after repeat administration of BIVV009.

FIG. 8 shows a schematic of a clinical trial protocol for administering BIVV009 to kidney transplant recipients diagnosed with late active ABMR associated with signs of donor-specific antibody (DSA)-triggered classical pathway (CP) activation. Index Bx, baseline biopsy; FU Bx, follow-up biopsy; EOS, end of study.

FIG. 9 shows individual DSA specificities in subjects participating in the clinical trial identified at the time of study inclusion.

FIG. 10A shows the relationship between median (interquartile range) serum concentration of BIVV009 (log scale) and overall % CP activity detected by WIESLAB® CP assay. FIG. 10B shows the effect of BIVV009 on C3d fixation triggered by the immunodominant donor-specific antibodies (DSA) on single bead assays or by a broad panel of third-party anti-HLA antibodies pre-coated to mixed beads (patient serum as complement source), and, in parallel, the IgG mean fluorescence intensity (MFI) of the immunodominant DSA and its capability to fix recombinant C1q.

FIG. 11A-11H show the effects of BIVV009 on morphologic and molecular biopsy results. FIG. 11A shows C4d staining in peritubular capillaries (C4d score). FIG. 11B shows the extent of microcirculation (g+ptc score). FIG. 11C shows the extent of transplant glomerulopathy (cg score). FIG. 11D shows the ABMR score. FIG. 11E shows the TCMR score. FIG. 11F shows the all rejection score. FIG. 11G shows the acute kidney injury (AKI) score. FIG. 11H shows the chronic injury (atrophy/fibrosis) score. Box plots represent the median, interquartile range and range. For statistical comparisons, the Wilcoxon rank test was used.

FIG. 12A-12L show the effect of BIVV009 on pathogenesis-based transcript (PBT) scores. Panels show differences in the expression of PBT between index and follow-up biopsies. FIG. 12A shows transcripts representative of T cell burden (TCB). FIG. 12B shows transcripts representative of cytotoxic T cell infiltration (QCAT). FIG. 12C shows transcripts representative of NK cell burden-associated transcripts (NKB). FIGS. 12D and 12E show transcripts representative of Macrophage-associated transcripts (QCMAT, AMAT1). FIG. 12F shows transcripts representative of gamma-interferon associated transcripts (GRIT1). FIG. 12G shows transcripts associated with the presence of DSA (DSAST). FIG. 12H shows transcripts associated with endothelial inflammation (ENDAT). FIG. 12I shows the response to DSA-associated transcripts (eDSAST). FIG. 12J shows transcripts associated with acute kidney injury and wound repair (IRRAT). FIGS. 12K and 12L show transcripts representative of healthy kidney tissue and normal function (KT1, KT2), respectively.

FIG. 13 shows the course of estimated glomerular filtration rate (eGFR, mL/min/1.73 m²) and urinary protein/creatinine (P/C) ratio (mg/g) in subjects over the study period of 50 days. Arrows indicate BIVV009 administration days.

FIG. 14 is a flow chart of a phase-1 clinical trial of healthy subjects treated with either BIVV009 (humanized anti-C1s monoclonal antibody) or a negative control. *Subject did not receive the second infusion in part B (multiple infusion) due to gastroenteritis; because of minimal variation in PK data after adjustment, the subject was not excluded from final analysis.

FIGS. 15A-15B are graphical representations of mean (+SE) serum concentrations of BIVV009 vs. time following a single 60 minutes iv infusion of BIVV009 in healthy volunteers (part A; (FIG. 15A), and mean (+SE) serum trough concentrations of BIVV009 vs. time following weekly 60 minutes iv infusions of BIVV009 (part B; FIG. 15B).

FIGS. 16A-16C are graphical representations of individual body weight vs AUClast D (μg*h/mL/mg) (FIG. 16A), Cₘₐₓ D (μg/mL/mg) (FIG. 16B), and half-life_Lambda_z (h) ((FIG. 16C). AUC: area under the concentration-time curve; MAD: multiple ascending doses; Cmax: maximum serum concentration; HL: half-life.

FIGS. 17A-17C are graphical representations of mean (+SE) serum classical complement pathway (CP) activity vs. time following a single 60 minutes iv infusion of BIVV009 in healthy volunteers (FIG. 17A), and mean (+SE) serum trough CP activity vs. time following single or weekly 60 minutes iv infusions of BIVV009 (FIG. 17B). FIG. 17C is a graphical representation of individual trough serum CP activity vs. time following multiple once-weekly 60 minutes iv infusions of BIVV009 in healthy volunteers.

FIG. 18 shows the expected body weight (kg) distribution in Phase 3 Trials. Simulations were based on 631 CAgD patients (mean (SD)=77.0 (19.7) kg, median (min-max)=74.8 (40.6-163.3) kg) that were extracted from a US electronic medical record and claims database.

FIG. 19 shows the simulated median (90% Prediction Interval (PI)) BIVV009 concentrations for the proposed dosing regimen. The solid line represents the median BIVV009 concentrations and the shaded region represents the 90% prediction interval. The dashed line represents the BIVV009 concentration for which target-mediated drug disposition (TMDD) starts to occur (100 μg/mL).

FIGS. 20A-20B show fluorescent microscope images of monkey esophageal tissue incubated with serum from a patient with bullous pemphigoid in the absence or presence of an anti-C1s antibody and stained for the presence of C3d. Fluorescence indicates C3d deposition of C3d on the cell surface. FIG. 20A shows the levels of C3d deposition in the absence of the anti-C1s antibody. FIG. 20B shows the levels of C3d deposition in the presence of the anti-C1s antibody.

FIGS. 21A-21C show fluorescent microscope images of patient skin biopsies in a bullous pemphigoid patient treated with BIVV009 antibody. Fluorescence indicates deposition of C3d at the dermal-epidermal junction. FIG. 21A shows the level of C3d deposition at the dermal-epidermal junction before BIVV009 treatment. FIG. 21B shows the level of C3d deposition at the dermal-epidermal junction during BIVV009 treatment. FIG. 21C shows the level of C3d deposition at the dermal-epidermal junction after BIVV009 treatment and antibody washout.

### DEFINITIONS

In order that the present disclosure can be more readily understood, certain terms are first defined. As used in this application, except as otherwise expressly provided herein, each of the following terms shall have the meaning set forth below. Additional definitions are set forth throughout the application.

The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of an disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

The term “about” as used in connection with a numerical value throughout the specification and the claims denotes an interval of accuracy, familiar and acceptable to a person skilled in the art. In general, such interval of accuracy is 10%.

It must be noted that as used herein and in the appended claims, the singular forms “a,” “an,” and “the” include plural referents unless the context clearly dictates otherwise. Thus, for example, reference to “an anti-C1s antibody” includes a plurality of such antibody and reference to “the complement-mediated disease” includes reference to one or more complement-mediated diseases and equivalents thereof known to those skilled in the art, and so forth. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, engineered antibodies, single-chain antibodies (scAb), single domain antibodies (dAb), single domain heavy chain antibodies, single domain light chain antibodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

For example, humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)).

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315(1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}—V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

The term “affinity” refers to the degree to which a binding molecule, e.g., an antibody, binds to an antigen so as to shift the equilibrium of antigen and binding molecule toward the presence of a complex formed by their binding. Thus, where an antigen and binding molecule are combined in relatively equal concentration, a binding molecule of high affinity will bind to the available antigen so as to shift the equilibrium toward high concentration of the resulting complex. Binding molecules, e.g., antibodies, or antigen-binding fragments, variants or derivatives thereof of the present disclosure can also be described or specified in terms of their binding affinity to an antigen. The affinity of binding molecule, e.g., an antibody, for an antigen can be determined experimentally using any suitable method. (See, e.g., Berzofsky et al., “Antibody-Antigen Interactions,” In Fundamental Immunology, Paul, W. E., Ed., Raven Press: New York, N.Y, (1984); Kuby, Janis Immunology, W. H. Freeman and Company: New York, N.Y. (1992); and methods described therein).

The measured affinity of a particular binding molecule-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of binding molecule and antigen, and a standardized buffer.

The “high affinity” for a binding molecule, e.g., an antibody, refers to an equilibrium association constant (K_{aff}) of at least about 1×10⁷ liters/mole, or at least about 1×10⁸ liters/mole, or at least about 1×10⁹ liters/mole, or at least about 1×10¹⁰ liters/mole, or at least about 1×10¹¹ liters/mole, or at least about 1×10¹² liters/mole, or at least about 1×10¹³ liters/mole, or at least about 1×10¹⁴ liters/mole or greater. “High affinity” binding can vary for antibody isotypes.

K_{D}, the equilibrium dissociation constant, is a term that is also used to describe antibody affinity and is the inverse of Kₐr. K_{D} is obtained from the ratio of k_{d} to kₐ (i.e., k_{d}/kₐ) and is expressed as a molar concentration (M). K_{D} values for antibodies can be determined using methods well established in the art. Available methods for determining the K_{D} of an antibody include a Bio-Layer Interferometry (BLI) assay, surface plasmon resonance, a biosensor system such as a BIACORE® system or flow cytometry and Scatchard analysis. If K_{D} is used, the term “high affinity” for an antibody refers to an equilibrium dissociation constant (K_{D}) of less than about 1×10⁻⁷ M, or less than about 1×10⁻⁸ M, or less than about 1×10⁻⁹ M, or less than about 1×10⁻¹⁰ M, or less than about 1×10⁻¹¹ M, or less than about 1×10⁻¹² M, or less than about 1×10¹³ M, less than about 1×10⁻¹⁴ M, or lower.

Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A subject anti-C1s antibody binds specifically to an epitope within a complement C1s protein. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The terms “compete” or “cross-compete”, as used herein with regard to a binding molecule, e.g., an antibody, means that a first binding molecule, e.g., a first antibody or an antigen-binding portion thereof, binds to an epitope in a manner sufficiently similar to the binding of a second binding molecule, e.g., a second antibody or an antigen-binding portion thereof, such that the result of binding of the first binding molecule with its cognate epitope is detectably decreased in the presence of the second binding molecule compared to the binding of the first binding molecule in the absence of the second binding molecule. The alternative, where the binding of the second binding molecule to its epitope is also detectably decreased in the presence of the first binding molecule, can, but need not be the case. That is, a first binding molecule can inhibit the binding of a second binding molecule to its epitope without that second molecule inhibiting the binding of the first binding molecule to its respective epitope. However, where each binding molecule detectably inhibits the binding of the other binding molecule with its cognate epitope, whether to the same, greater, or lesser extent, the binding molecules are said to “cross-compete” with each other for binding of their respective epitope(s). Both competing and cross-competing binding molecules are encompassed by the present disclosure.

Binding molecules, e.g., antibodies, are said to “bind to the same epitope” or “comprising the same binding site” or have “essentially the same binding” characteristics, if the binding molecules cross-compete so that only one antibody can bind to the epitope at a given point of time, i.e., one binding molecule prevents the binding or modulating effect of the other.

Competition herein means a greater relative inhibition than at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as determined by competition ELISA analysis or by ForteBio analysis, e.g., as described in the Examples section. It can be desirable to set a higher threshold of relative inhibition as criteria of what is a suitable level of competition in a particular context. Thus, for example, it is possible to set criteria for the competitive binding, wherein at least about 40% relative inhibition is detected, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or even about 100%, before an antibody is considered sufficiently competitive.

The term “epitope” as used herein refers to an antigenic protein determinant (e.g., an amino acid subsequence of C1s) capable of binding to a binding molecule, e.g., an antibody. Epitopes usually consist of chemically active surface groupings of molecules such as amino acids or sugar side chains and usually have specific three dimensional structural characteristics, as well as specific charge characteristics. The part of an antibody or binding molecule that recognizes the epitope is called a paratope. The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the 3-D surface features and shape or tertiary structure of the antigen. By contrast, linear epitopes interact with the paratope based on their primary structure. A linear epitope is formed by a continuous sequence of amino acids from the antigen.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 1 as a comparison. The CDRs provided in the present disclosure were defined in accordance with Kabat 1991.

TABLE 1
CDR Definitions

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

An “isolated” antibody is one that has been identified and separated and/or recovered from a component of its natural environment. Contaminant components of its natural environment are materials that would interfere with diagnostic or therapeutic uses for the antibody, and can include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; (c) relieving the disease, e.g., causing regression of the disease; and (d) relieving or reducing symptoms associated with the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. Also encompassed by these terms are any animal that has a complement system, such as mammals, fish, and some invertebrates. As such these terms include complement system-containing mammal, fish, and invertebrate companion animals, agricultural animals, work animals, zoo animals, and lab animals.

A “therapeutically effective amount,” “efficacious amount,” or “effective dose” refers to the amount of an anti-complement C1s antibody that, when administered to a mammal or other subject for treating a disease, is sufficient to effect such treatment for the disease.

The term “less than” (<) means a value that is less than, but not equal to, a reference value. The term “greater than” (>) means a value that is greater than, but not equal to, a reference value. The term “less than or equal to” (≤) means a value that is less than or equal to a reference value. The term “greater than or equal to” (≥) means a value that is greater than or equal to a reference value.

Before the present disclosure is further described, it is to be understood that this disclosure is not limited to particular embodiments described, as such can, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure.

Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided can be different from the actual publication dates which can need to be independently confirmed.

### DETAILED DESCRIPTION

The present disclosure provides methods of treating a complement-mediated disease or disorder in an individual, and methods of inhibiting activation of complement component C4 in an individual in need thereof. In some aspects, the methods comprise administering to the individual an anti-C1s antibody in a fixed dose of 5.5 g. In some aspects, the methods comprise administering to the individual an anti-C1s antibody in a fixed dose between about 4.0 g and about 10.0 g, e.g., about 4 g, about 4.5 g, about 5 g, about 5.5 g, about 6 g, about 6.5 g, about 7.5 g, about 8 g, about 8.5 g, about 9 g, about 9.5 g, or about 10 g. In some aspects, the methods comprise administering to the individual an effective dose of an anti-C1s antibody, where the serum concentration of the antibody is between about 20 μg/ml and about 150 μg/ml.

### Anti-C1s Antibody

An anti-C1s antibody suitable for the present disclosure specifically binds a conformational epitope within amino acids 272-422 of the following amino acid sequence of human C1s:

(SEQ ID NO: 9)
EPTMYGEILSPNYPQAYPSEVEKSWDIEVPEGYGIHLYFTHLDIELSENC
AYDSVQIISGDTEEGRLCGQRSSNNPHSPIVEEFQVPYNKLQVIFKSDFS
NEERFTGFAAYYVATDINECTDEVDVPCSHFCNNFIGGYFCSCPPEYFLH
DDMKNCGVNCSGDVFTALIGEIASPNYPKPYPENSRCEYQIRLEKGFQVV
VTLRREDFDVEAADSAGNCLDSLVEVAGDRQFGPYCGHGFPGPLNIETKS
NALDIIFQTDLTGQKKGWKLRYHGDPMPCPKEDTPNSVWEPAKAKYVERD
VVQITCLDGFEVVEGRVGATSFYSTCQSNGKWSNSKLKCQPVDCGIPESI
ENGKVEDPESTLFGSVIRYTCEEPYYYMENGGGGEYHCAGNGSWVNEVLG
PELPKCVPVCGVPREPFEEKQRIIGGSDADIKNFPWQVFEDNPWAGGALI
NEYWVLTAAHVVEGNREPTMYVGSTSVQTSRLAKSKMLTPEHVFIHPGWK
LLEVPEGRTNEDNDIALVRLKDPVKMGPTVSPICLPGTSSDYNLMDGDLG
LISGWGRTEKRDRAVRLKAARLPVAPLRKCKEVKVEKPTADAEAYVFTPN
MICAGGEKGMDSCKGDSGGAFAVQDPNDKTKFYAAGLVSWGPQCGTYGLY
TRVKNYVDWIMKTMQENSTPRED

An anti-C1s antibody suitable for the present disclosure inhibits C1s-mediated cleavage of complement component C4. In some cases, an anti-C1s antibody suitable for use in a method of the present disclosure inhibits C1s-mediated cleavage of complement component C4, but does not inhibit C1s-mediated cleavage of complement component C2. In some cases, the antibody inhibits a component of the classical complement pathway; in some cases, the classical complement pathway component is C1s. In some instances, the antibody does not inhibit protease activity of C1s.

In some cases, an anti-C1s antibody suitable for the present disclosure is humanized. In some cases, the anti-C1s antibody comprises a humanized light-chain framework region. In some cases, the anti-C1s antibody comprises a humanized heavy-chain framework region. In some cases, the anti-C1s antibody comprises a humanized light-chain framework region and a humanized heavy-chain framework region. In some cases, an anti-C1s antibody suitable for the present disclosure is a humanized monoclonal antibody.

Humanization of a framework region(s) reduces the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of the therapy. The HAMA response is measured, for example, by detecting antibodies to the humanized therapeutic reagent, in serum samples from the patient using a method known to one in the art, including surface plasmon resonance technology (BIACORE) and/or solid-phase enzyme-linked immunosorbent assay (ELISA) analysis. In many cases, a subject humanized anti-C1s antibody does not substantially elicit a HAMA response in a human subject.

Certain amino acids from the human variable region framework residues are selected for substitution based on their possible influence on CDR conformation and/or binding antigen. The unnatural juxtaposition of murine CDR regions with human variable framework region can result in unnatural conformational restraints, which, unless corrected by substitution of certain amino acid residues, lead to loss of binding affinity.

The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, e.g., those sharing at least 60%, at least 70%, at least 80%, at least 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

CDR and framework regions are as defined by Kabat, Sequences of Proteins of Immunological Interest (National Institutes of Health, Bethesda, Md., 1987 and 1991). An alternative structural definition has been proposed by Chothia et al., J. Mol. Biol. 196:901 (1987); Nature 342:878 (1989); and J. Mol. Biol. 186:651 (1989) (collectively referred to as “Chothia”). When framework residues, as defined by Kabat, supra, constitute structural loop residues as defined by Chothia, supra, the amino acids present in the mouse antibody can be selected for substitution into the humanized antibody. Residues that are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk J M B 196:901 (1987)). These amino acids are particularly likely to interact with the amino acids in the CDRs and, if chosen from the acceptor, to distort the donor CDRs and reduce affinity. Moreover, the adjacent amino acids can interact directly with the antigen (Amit et al., Science, 233:747 (1986)) and selecting these amino acids from the donor can be desirable to keep all the antigen contacts that provide affinity in the original antibody.

In some cases, an anti-C1s antibody suitable for the present disclosure comprises a light chain region variable region (VL) comprising CDR-L1, CDR-L2, and CDR-L3 present in a VL comprising the amino acid sequence of SEQ ID NO: 7.

SEQ ID NO: 7:
QIVLTQSPAIMSASLGERVTMTCTASSSVSSSYLHWYQQKPGSSPKLWIY
STSNLASGVPARFSGSGSGTFYSLTISSMEAEDDATYYCHQYYRLPPITF
GAGTKLELK.

In some cases, an anti-C1s antibody suitable for the present disclosure comprises a heavy chain region variable region (VH) comprising CDR-H1, CDR-H2, and CDR-H3 present in a VH comprising the amino acid sequence of SEQ ID NO: 8.

SEQ ID NO: 8:
EVMLVESGGALVKPGGSLKLSCAASGETFSNYAMSWVRQIPEKRLEWVAT
ISSGGSHTYYLDSVKGRFTISRDNARDTLYLQMSSLRSEDTALYYCARLF
TGYAMDYWGQGTSVTVSS.

In some cases, an anti-C1s antibody suitable for the present disclosure comprises: a) a light chain region comprising CDR-L1, CDR-L2, and CDR-L3 having the amino acid sequences set forth in SEQ ID NO:1, SEQ ID NO:2, and SEQ ID NO:3, respectively; and b) a heavy chain region comprising CDR-H1, CDR-H2, and CDR-H3 having the amino acid sequences set forth in SEQ ID NO:4, SEQ ID NO:5, and SEQ ID NO:6, respectively. In some of these embodiments, the anti-C1s antibody includes a humanized V_{H} and/or V_{L} framework region.

SEQ ID NO: 1:
SSVSSSYLHWYQ;
SEQ ID NO: 2:
STSNLASGVP;
SEQ ID NO: 3:
HQYYRLPPIT;
SEQ ID NO: 4:
GFTFSNYAMSWV;
SEQ ID NO: 5:
ISSGGSHTYY;
SEQ ID NO: 6:
ARLFTGYAMDY.

In some cases, an anti-C1s antibody suitable for the present disclosure comprises: a) a light chain region comprising CDR-L1, CDR-L2, and CDR-L3 having the amino acid sequences set forth in SEQ ID NO:10, SEQ ID NO:11, and SEQ ID NO:3, respectively; and b) a heavy chain region comprising CDR-H1, CDR-H2, and CDR-H3 having the amino acid sequences set forth in SEQ ID NO:12, SEQ ID NO:13, and SEQ ID NO:14, respectively. In some of these embodiments, the anti-C1s antibody includes a humanized V_{H} and/or V_{L} framework region.

SEQ ID NO: 10:
TASSSVSSSYLH;
SEQ ID NO: 11:
STSNLAS;
SEQ ID NO: 3:
HQYYRLPPIT;
SEQ ID NO: 12:
NYAMS;
SEQ ID NO: 13:
TISSGGSHTYYLDSVKG;
SEQ ID NO: 14:
LFTGYAMDY.

In some cases, an anti-C1s antibody suitable for the present disclosure comprises a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:15.

SEQ ID NO: 15:
QIVLTQSPAILSLSPGERATMSCTASSSVSSSYLHWYQQKPGKAPKLWIY
STSNLASGVPSRFSGSGSGTFYTLTISSLQAEDFATYYCHQYYRLPPITF
GQGTKLEIK.

In some cases, an anti-C1s antibody suitable for the present disclosure comprises a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:16.

SEQ ID NO: 16.
QIVLTQSPATLSLSPGERATMSCTASSSVSSSYLHWYQQKPGKAPKLWIY
STSNLASGVPSRFSGSGSGTDYTLTISSLQPEDFATYYCHQYYRLPPITF
GQGTKLEIK.

In some cases, an anti-C1s antibody suitable for the present disclosure comprises a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:17.

SEQ ID NO: 17:
QIVLTQSPATLSLSPGERATLSCTASSSVSSSYLHWYQQKPGKAPKLWIY
STSNLASGVPSRFSGSGSGTDYTLTISSLQPEDFATYYCHQYYRLPPITF
GQGTKLEIK.

In some cases, an anti-C1s antibody suitable for the present disclosure comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:18.

SEQ ID NO: 18:
EVMLVESGGGLVKPGGSLRLSCAASGETFSNYAMSWVRQAPGKGLEWVAT
ISSGGSHTYYLDSVKGRFTISRDNSKDTLYLQMSSLRAEDTALYYCARLF
TGYAMDYWGQGTSVTVSS

In some cases, an anti-C1s antibody suitable for the present disclosure comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:19.

SEQ ID NO: 19:
EVMLVESGGGLVKPGGSLRLSCAASGETFSNYAMSWVRQAPGKGLEWVAT
ISSGGSHTYYLDSVKGRFTISRDNSKDTLYLQMNSLRAEDTALYYCARLF
TGYAMDYWGQGTLVTVSS

In some cases, an anti-C1s antibody suitable for the present disclosure comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:20.

SEQ ID NO: 20:
EVMLVESGGGLVKPGGSLRLSCAASGETFSNYAMSWVRQAPGKGLEWVAT
ISSGGSHTYYLDSVKGRFTISRDNSKDTLYLQMSSLRAEDTALYYCARLF
TGYAMDYWGQGTSVTVSS

In some cases, an anti-C1s antibody suitable for the present disclosure comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:21.

SEQ ID NO: 21:
EVQLVESGGGLVKPGGSLRLSCAASGETFSNYAMSWVRQAPGKGLEWVAT
ISSGGSHTYYLDSVKGRFTISRDNSKNTLYLQMNSLRAEDTALYYCARLF
TGYAMDYWGQGTLVTVSS

In some cases, an anti-C1s antibody suitable for the present disclosure comprises a VL region comprising the amino acid sequence set forth in SEQ ID NO:15; and a VH region comprising the amino acid sequence set forth in SEQ ID NO:18.

In some cases, an anti-C1s antibody suitable for the present disclosure comprises a VL region comprising the amino acid sequence set forth in SEQ ID NO:15; and a VH region comprising the amino acid sequence set forth in SEQ ID NO:19.

In some cases, an anti-C1s antibody suitable for the present disclosure comprises a VL region comprising the amino acid sequence set forth in SEQ ID NO:15; and a VH region comprising the amino acid sequence set forth in SEQ ID NO:20.

In some cases, an anti-C1s antibody suitable for the present disclosure comprises a VL region comprising the amino acid sequence set forth in SEQ ID NO:15; and a VH region comprising the amino acid sequence set forth in SEQ ID NO:21.

In some cases, an anti-C1s antibody suitable for the present disclosure comprises a VL region comprising the amino acid sequence set forth in SEQ ID NO:16; and a VH region comprising the amino acid sequence set forth in SEQ ID NO:18.

In some cases, an anti-C1s antibody suitable for the present disclosure comprises a VL region comprising the amino acid sequence set forth in SEQ ID NO:16; and a VH region comprising the amino acid sequence set forth in SEQ ID NO:19.

In some cases, an anti-C1s antibody suitable for the present disclosure comprises a VL region comprising the amino acid sequence set forth in SEQ ID NO:16; and a VH region comprising the amino acid sequence set forth in SEQ ID NO:20.

In some cases, an anti-C1s antibody suitable for the present disclosure comprises a VL region comprising the amino acid sequence set forth in SEQ ID NO:16; and a VH region comprising the amino acid sequence set forth in SEQ ID NO:21.

In some cases, an anti-C1s antibody suitable for the present disclosure comprises a VL region comprising the amino acid sequence set forth in SEQ ID NO:17; and a VH region comprising the amino acid sequence set forth in SEQ ID NO:18.

In some cases, an anti-C1s antibody suitable for the present disclosure comprises a VL region comprising the amino acid sequence set forth in SEQ ID NO:17; and a VH region comprising the amino acid sequence set forth in SEQ ID NO:19.

In some cases, an anti-C1s antibody suitable for the present disclosure comprises a VL region comprising the amino acid sequence set forth in SEQ ID NO:17; and a VH region comprising the amino acid sequence set forth in SEQ ID NO:20.

In some cases, an anti-C1s antibody suitable for the present disclosure comprises a VL region comprising the amino acid sequence set forth in SEQ ID NO:17; and a VH region comprising the amino acid sequence set forth in SEQ ID NO:21.

In some embodiments, an anti-C1s antibody suitable for the present disclosure is an antibody that cross competes with a reference antibody. In one embodiment, the reference antibody comprises:
a) a VL region comprising the amino acid sequence set forth in SEQ ID NO:15; and a VH region comprising the amino acid sequence set forth in SEQ ID NO:18;
b) a VL region comprising the amino acid sequence set forth in SEQ ID NO:15; and a VH region comprising the amino acid sequence set forth in SEQ ID NO:19;
c) a VL region comprising the amino acid sequence set forth in SEQ ID NO:15; and a VH region comprising the amino acid sequence set forth in SEQ ID NO:20;
d) a VL region comprising the amino acid sequence set forth in SEQ ID NO:15; and a VH region comprising the amino acid sequence set forth in SEQ ID NO:21;
e) a VL region comprising the amino acid sequence set forth in SEQ ID NO:16; and a VH region comprising the amino acid sequence set forth in SEQ ID NO:18;
f) a VL region comprising the amino acid sequence set forth in SEQ ID NO:16; and a VH region comprising the amino acid sequence set forth in SEQ ID NO:19;
g) a VL region comprising the amino acid sequence set forth in SEQ ID NO:16; and a VH region comprising the amino acid sequence set forth in SEQ ID NO:20;
h) a VL region comprising the amino acid sequence set forth in SEQ ID NO:16; and a VH region comprising the amino acid sequence set forth in SEQ ID NO:21;
i) a VL region comprising the amino acid sequence set forth in SEQ ID NO:17; and a VH region comprising the amino acid sequence set forth in SEQ ID NO:18;
j) a VL region comprising the amino acid sequence set forth in SEQ ID NO:17; and a VH region comprising the amino acid sequence set forth in SEQ ID NO:19;
k) a VL region comprising the amino acid sequence set forth in SEQ ID NO:17; and a VH region comprising the amino acid sequence set forth in SEQ ID NO:20; or
l) a VL region comprising the amino acid sequence set forth in SEQ ID NO:17; and a VH region comprising the amino acid sequence set forth in SEQ ID NO:21.

In some cases, an anti-C1s antibody suitable for the present disclosure cross competes with an antibody comprising a light chain region variable region (VL) comprising CDR-L1, CDR-L2, and CDR-L3 present in a VL comprising the amino acid sequence of SEQ ID NO: 7.

In some cases, an anti-C1s antibody suitable for the present disclosure cross competes with an antibody comprising a heavy chain region variable region (VH) comprising CDR-H1, CDR-H2, and CDR-H3 present in a VH comprising the amino acid sequence of SEQ ID NO: 8.

In some cases, an anti-C1s antibody suitable for the present disclosure cross competes with an antibody comprising: a) a light chain region comprising CDR-L1, CDR-L2, and CDR-L3 having the amino acid sequences set forth in SEQ ID NO:1, SEQ ID NO:2, and SEQ ID NO:3, respectively; and b) a heavy chain region comprising CDR-H1, CDR-H2, and CDR-H3 having the amino acid sequences set forth in SEQ ID NO:4, SEQ ID NO:5, and SEQ ID NO:6, respectively. In some of these embodiments, the anti-C1s antibody includes a humanized V_{H} and/or V_{L} framework region.

In some cases, an anti-C1s antibody suitable for the present disclosure cross competes with an antibody comprising: a) a light chain region comprising CDR-L1, CDR-L2, and CDR-L3 having the amino acid sequences set forth in SEQ ID NO:10, SEQ ID NO:11, and SEQ ID NO:3, respectively; and b) a heavy chain region comprising CDR-H1, CDR-H2, and CDR-H3 having the amino acid sequences set forth in SEQ ID NO:12, SEQ ID NO:13, and SEQ ID NO:14, respectively. In some of these embodiments, the anti-C1s antibody includes a humanized V_{H} and/or V_{L} framework region.

In other embodiments, an anti-C1s antibody suitable for the present disclosure is an antibody that specifically binds to the same epitope as a reference antibody. In one embodiment, the reference antibody comprises:
a) a VL region comprising the amino acid sequence set forth in SEQ ID NO:15; and a VH region comprising the amino acid sequence set forth in SEQ ID NO:18;
b) a VL region comprising the amino acid sequence set forth in SEQ ID NO:15; and a VH region comprising the amino acid sequence set forth in SEQ ID NO:19;
c) a VL region comprising the amino acid sequence set forth in SEQ ID NO:15; and a VH region comprising the amino acid sequence set forth in SEQ ID NO:20;
d) a VL region comprising the amino acid sequence set forth in SEQ ID NO:15; and a VH region comprising the amino acid sequence set forth in SEQ ID NO:21;
e) a VL region comprising the amino acid sequence set forth in SEQ ID NO:16; and a VH region comprising the amino acid sequence set forth in SEQ ID NO:18;
f) a VL region comprising the amino acid sequence set forth in SEQ ID NO:16; and a VH region comprising the amino acid sequence set forth in SEQ ID NO:19;
g) a VL region comprising the amino acid sequence set forth in SEQ ID NO:16; and a VH region comprising the amino acid sequence set forth in SEQ ID NO:20;
h) a VL region comprising the amino acid sequence set forth in SEQ ID NO:16; and a VH region comprising the amino acid sequence set forth in SEQ ID NO:21;
i) a VL region comprising the amino acid sequence set forth in SEQ ID NO:17; and a VH region comprising the amino acid sequence set forth in SEQ ID NO:18;
j) a VL region comprising the amino acid sequence set forth in SEQ ID NO:17; and a VH region comprising the amino acid sequence set forth in SEQ ID NO:19;
k) a VL region comprising the amino acid sequence set forth in SEQ ID NO:17; and a VH region comprising the amino acid sequence set forth in SEQ ID NO:20; or
l) a VL region comprising the amino acid sequence set forth in SEQ ID NO:17; and a VH region comprising the amino acid sequence set forth in SEQ ID NO:21.

In some cases, an anti-C1s antibody suitable for the present disclosure specifically binds to the same epitope as an antibody comprising a light chain region variable region (VL) comprising CDR-L1, CDR-L2, and CDR-L3 present in a VL comprising the amino acid sequence of SEQ ID NO: 7.

In some cases, an anti-C1s antibody suitable for the present disclosure specifically binds to the same epitope as an antibody comprising a heavy chain region variable region (VH) comprising CDR-H1, CDR-H2, and CDR-H3 present in a VH comprising the amino acid sequence of SEQ ID NO: 8.

In some cases, an anti-C1s antibody suitable for the present disclosure specifically binds to the same epitope as an antibody comprising: a) a light chain region comprising CDR-L1, CDR-L2, and CDR-L3 having the amino acid sequences set forth in SEQ ID NO:1, SEQ ID NO:2, and SEQ ID NO:3, respectively; and b) a heavy chain region comprising CDR-H1, CDR-H2, and CDR-H3 having the amino acid sequences set forth in SEQ ID NO:4, SEQ ID NO:5, and SEQ ID NO:6, respectively. In some of these embodiments, the anti-C1s antibody includes a humanized V_{H} and/or V_{L} framework region.

In some cases, an anti-C1s antibody suitable for the present disclosure specifically binds to the same epitope as an antibody comprising: a) a light chain region comprising CDR-L1, CDR-L2, and CDR-L3 having the amino acid sequences set forth in SEQ ID NO:10, SEQ ID NO:11, and SEQ ID NO:3, respectively; and b) a heavy chain region comprising CDR-H1, CDR-H2, and CDR-H3 having the amino acid sequences set forth in SEQ ID NO:12, SEQ ID NO:13, and SEQ ID NO:14, respectively. In some of these embodiments, the anti-C1s antibody includes a humanized V_{H} and/or V_{L} framework region.

In some cases, an anti-C1s antibody suitable for the present disclosure comprises a light chain variable region comprising an amino acid sequence at least about 85%, 86%, 87%, 88%, 89%, 90%, 91%, 92%, 93%, 94%, 95%, 96%, 97%, 98% or 99% identical to the amino acid sequence set forth in SEQ ID NO:15.

In some cases, an anti-C1s antibody suitable for the present disclosure comprises a light chain variable region comprising an amino acid sequence that is at least about 85%, 86%, 87%, 88%, 89%, 90%, 91%, 92%, 93%, 94%, 95%, 96%, 97%, 98% or 99% identical to the amino acid sequence set forth in SEQ ID NO:16.

In some cases, an anti-C1s antibody suitable for the present disclosure comprises a light chain variable region comprising an amino acid sequence at least about 85%, 86%, 87%, 88%, 89%, 90%, 91%, 92%, 93%, 94%, 95%, 96%, 97%, 98% or 99% identical to the amino acid sequence set forth in SEQ ID NO:17.

In some cases, an anti-C1s antibody suitable for the present disclosure comprises a heavy chain variable region comprising an amino acid sequence at least about 85%, 86%, 87%, 88%, 89%, 90%, 91%, 92%, 93%, 94%, 95%, 96%, 97%, 98% or 99% identical to the amino acid sequence set forth in SEQ ID NO:18.

In some cases, an anti-C1s antibody suitable for the present comprises a heavy chain variable region comprising an amino acid sequence at least about 85%, 86%, 87%, 88%, 89%, 90%, 91%, 92%, 93%, 94%, 95%, 96%, 97%, 98% or 99% identical to the amino acid sequence set forth in SEQ ID NO:19.

In some cases, an anti-C1s antibody suitable for the present disclosure comprises a heavy chain variable region comprising an amino acid sequence at least about 85%, 86%, 87%, 88%, 89%, 90%, 91%, 92%, 93%, 94%, 95%, 96%, 97%, 98% or 99% identical to the amino acid sequence set forth in SEQ ID NO:20.

In some cases, an anti-C1s antibody suitable for the present disclosure comprises a heavy chain variable region comprising an amino acid sequence at least about 85%, 86%, 87%, 88%, 89%, 90%, 91%, 92%, 93%, 94%, 95%, 96%, 97%, 98% or 99% identical to the amino acid sequence set forth in SEQ ID NO:21.

In some cases, an anti-C1s antibody suitable for the present disclosure cross competes with an antibody comprising a VL region comprising the amino acid sequence set forth in SEQ ID NO:15; and a VH region comprising the amino acid sequence set forth in SEQ ID NO:18.

In some cases, an anti-C1s antibody suitable for the present disclosure cross competes with an antibody comprising a VL region comprising the amino acid sequence set forth in SEQ ID NO:15; and a VH region comprising the amino acid sequence set forth in SEQ ID NO:19.

In some cases, an anti-C1s antibody suitable for the present disclosure cross competes with an antibody comprising a VL region comprising the amino acid sequence set forth in SEQ ID NO:15; and a VH region comprising the amino acid sequence set forth in SEQ ID NO:20.

In some cases, an anti-C1s antibody suitable for the present disclosure cross competes with an antibody comprising a VL region comprising the amino acid sequence set forth in SEQ ID NO:15; and a VH region comprising the amino acid sequence set forth in SEQ ID NO:21.

In some cases, an anti-C1s antibody suitable for the present disclosure cross competes with an antibody comprising a VL region comprising the amino acid sequence set forth in SEQ ID NO:16; and a VH region comprising the amino acid sequence set forth in SEQ ID NO:18.

In some cases, an anti-C1s antibody suitable for the present disclosure cross competes with an antibody comprising a VL region comprising the amino acid sequence set forth in SEQ ID NO:16; and a VH region comprising the amino acid sequence set forth in SEQ ID NO:19.

In some cases, an anti-C1s antibody suitable for the present disclosure cross competes with an antibody comprising a VL region comprising the amino acid sequence set forth in SEQ ID NO:16; and a VH region comprising the amino acid sequence set forth in SEQ ID NO:20.

In some cases, an anti-C1s antibody suitable for the present disclosure cross competes with an antibody comprising a VL region comprising the amino acid sequence set forth in SEQ ID NO:16; and a VH region comprising the amino acid sequence set forth in SEQ ID NO:21.

In some cases, an anti-C1s antibody suitable for the present disclosure cross competes with an antibody comprising a VL region comprising the amino acid sequence set forth in SEQ ID NO:17; and a VH region comprising the amino acid sequence set forth in SEQ ID NO:18.

In some cases, an anti-C1s antibody suitable for the present disclosure cross competes with an antibody comprising a VL region comprising the amino acid sequence set forth in SEQ ID NO:17; and a VH region comprising the amino acid sequence set forth in SEQ ID NO:19.

In some cases, an anti-C1s antibody suitable for the present disclosure cross competes with an antibody comprising a VL region comprising the amino acid sequence set forth in SEQ ID NO:17; and a VH region comprising the amino acid sequence set forth in SEQ ID NO:20.

In some cases, an anti-C1s antibody suitable for the present disclosure cross competes with an antibody comprising a VL region comprising the amino acid sequence set forth in SEQ ID NO:17; and a VH region comprising the amino acid sequence set forth in SEQ ID NO:21.

In some cases, an anti-C1s antibody suitable for the present disclosure binds the same epitope as an antibody comprising a light chain region variable region (VL) comprising CDR-L1, CDR-L2, and CDR-L3 present in a VL comprising the amino acid sequence of SEQ ID NO: 7.

In some cases, an anti-C1s antibody suitable for the present disclosure binds the same epitope as an antibody comprising a heavy chain region variable region (VH) comprising CDR-H1, CDR-H2, and CDR-H3 present in a VH comprising the amino acid sequence of SEQ ID NO: 8.

In some cases, an anti-C1s antibody suitable for the present disclosure binds the same epitope as an antibody comprising: a) a light chain region comprising CDR-L1, CDR-L2, and CDR-L3 having the amino acid sequences set forth in SEQ ID NO:1, SEQ ID NO:2, and SEQ ID NO:3, respectively; and b) a heavy chain region comprising CDR-H1, CDR-H2, and CDR-H3 having the amino acid sequences set forth in SEQ ID NO:4, SEQ ID NO:5, and SEQ ID NO:6, respectively. In some of these embodiments, the anti-C1s antibody includes a humanized V_{H} and/or V_{L} framework region.

In some cases, an anti-C1s antibody suitable for the present disclosure binds the same epitope as an antibody comprising: a) a light chain region comprising CDR-L1, CDR-L2, and CDR-L3 having the amino acid sequences set forth in SEQ ID NO:10, SEQ ID NO:11, and SEQ ID NO:3, respectively; and b) a heavy chain region comprising CDR-H1, CDR-H2, and CDR-H3 having the amino acid sequences set forth in SEQ ID NO:12, SEQ ID NO:13, and SEQ ID NO:14, respectively. In some of these embodiments, the anti-C1s antibody includes a humanized V_{H} and/or V_{L} framework region.

In some cases, an anti-C1s antibody suitable for the present disclosure binds the same epitope as an antibody comprising a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:15.

In some cases, an anti-C1s antibody suitable for the present disclosure binds the same epitope as an antibody comprising a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:16.

In some cases, an anti-C1s antibody suitable for the present disclosure binds the same epitope as an antibody comprising a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:17.

In some cases, an anti-C1s antibody suitable for the present disclosure binds the same epitope as an antibody comprising a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:18.

In some cases, an anti-C1s antibody suitable for the present disclosure binds the same epitope as an antibody comprising a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:19.

In some cases, an anti-C1s antibody suitable for the present disclosure binds the same epitope as an antibody comprising a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:20.

In some cases, an anti-C1s antibody suitable for the present disclosure binds the same epitope as an antibody comprising a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:21.

In some cases, an anti-C1s antibody suitable for the present disclosure binds the same epitope as an antibody comprising a VL region comprising the amino acid sequence set forth in SEQ ID NO:15; and a VH region comprising the amino acid sequence set forth in SEQ ID NO:18. In some cases, an anti-C1s antibody suitable for the present disclosure binds the same epitope as an antibody comprising a VL region comprising the amino acid sequence set forth in SEQ ID NO:15; and a VH region comprising the amino acid sequence set forth in SEQ ID NO:19. In some cases, an anti-C1s antibody suitable for the present disclosure binds the same epitope as an antibody comprising a VL region comprising the amino acid sequence set forth in SEQ ID NO:15; and a VH region comprising the amino acid sequence set forth in SEQ ID NO:20. In some cases, an anti-C1s antibody suitable for the present disclosure binds the same epitope as an antibody comprising a VL region comprising the amino acid sequence set forth in SEQ ID NO:15; and a VH region comprising the amino acid sequence set forth in SEQ ID NO:21. In some cases, an anti-C1s antibody suitable for the present disclosure binds the same epitope as an antibody comprising a VL region comprising the amino acid sequence set forth in SEQ ID NO:16; and a VH region comprising the amino acid sequence set forth in SEQ ID NO:18. In some cases, an anti-C1s antibody suitable for the present disclosure binds the same epitope as an antibody comprising a VL region comprising the amino acid sequence set forth in SEQ ID NO:16; and a VH region comprising the amino acid sequence set forth in SEQ ID NO:19. In some cases, an anti-C1s antibody suitable for the present disclosure binds the same epitope as an antibody comprising a VL region comprising the amino acid sequence set forth in SEQ ID NO:16; and a VH region comprising the amino acid sequence set forth in SEQ ID NO:20. In some cases, an anti-C1s antibody suitable for the present disclosure binds the same epitope as an antibody comprising a VL region comprising the amino acid sequence set forth in SEQ ID NO:16; and a VH region comprising the amino acid sequence set forth in SEQ ID NO:21. In some cases, an anti-C1s antibody suitable for the present disclosure binds the same epitope as an antibody comprising a VL region comprising the amino acid sequence set forth in SEQ ID NO:17; and a VH region comprising the amino acid sequence set forth in SEQ ID NO:18. In some cases, an anti-C1s antibody suitable for the present disclosure binds the same epitope as an antibody comprising a VL region comprising the amino acid sequence set forth in SEQ ID NO:17; and a VH region comprising the amino acid sequence set forth in SEQ ID NO:19. In some cases, an anti-C1s antibody suitable for the present disclosure binds the same epitope as an antibody comprising a VL region comprising the amino acid sequence set forth in SEQ ID NO:17; and a VH region comprising the amino acid sequence set forth in SEQ ID NO:20. In some cases, an anti-C1s antibody suitable for the present disclosure binds the same epitope as an antibody comprising a VL region comprising the amino acid sequence set forth in SEQ ID NO:17; and a VH region comprising the amino acid sequence set forth in SEQ ID NO:21.

In some embodiments, an anti-C1s antibody suitable for the present disclosure is BIVV009. BIVV009 comprises a heavy chain comprising the amino acid sequence set forth in SEQ ID NO: 22 and a light chain region comprising the amino acid sequence set forth in SEQ ID NO: 23.

Heavy Chain of BIVV009:
SEQ ID NO: 22
EVQLVESGGGLVKPGGSLRLSCAASGFTFSNYAMSWVRQAPGKGLEWVAT
ISSGGSHTYYLDSVKGRFTISRDNSKNTLYLQMNSLRAEDTALYYCARLF
TGYAMDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Light Chain of BIVV009:
SEQ ID NO: 23
QIVLTQSPATLSLSPGERATMSCTASSSVSSSYLHWYQQKPGKAPKLWIY
STSNLASGVPSRFSGSGSGTDYTLTISSLQPEDFATYYCHQYYRLPPITF
GQGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC

In some embodiments, an anti-C1s antibody suitable for the present disclosure is selected from the group consisting of an Ig monomer, a Fab fragment, a F(ab′)₂ fragment, a Fd fragment, a scFv, a scAb, a dAb, a Fv, a single domain heavy chain antibody, and a single domain light chain antibody.

In some cases, an anti-C1s antibody suitable for the present disclosure comprises a constant region of an immunoglobulin (e.g., an Fc region). The Fc region, if present, can be a human Fc region or an Fc region from any animal that has a complement system. In some embodiments, the Fc region, if present, is a human Fc region. If constant regions are present, the antibody can contain both light chain and heavy chain constant regions. Suitable heavy chain constant region include CH1, hinge, CH2, CH3, and CH4 regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Another example of a suitable heavy chain Fc region is a human isotype IgG2 Fc. Yet another example of a suitable heavy chain Fc region is a human isotype IgG3 Fc. Light chain constant regions can be lambda or kappa. An anti-C1s antibody suitable for use in a method of the present disclosure can comprise sequences from more than one class or isotype. Antibodies can be expressed as tetramers containing two light and two heavy chains, as separate heavy chains, light chains, as Fab, Fab′, F(ab′)₂, and Fv, or as single chain antibodies in which heavy and light chain variable domains are linked through a spacer.

In some cases, the heavy chain region is of the isotype IgG4. In some of these embodiments, the hinge region comprises an S241P substitution. See, e.g., Angal et al. (1993) Mol. Immunol. 30:105. In some of these embodiments, the hinge region comprises an L236E (or L235E, using EU numbering; Kabat et al. (1991) Sequences of Proteins of Immunological Interest, 5^{th} Ed. U.S. Dept. Health and Human Services, Bethesda, Md., NIH Publication No. 91-3242) substitution. See, e.g., Reddy et al. (2000) J. Immunol. 164:1925; and Klechevsky et al. (2010) Blood 116:1685. In some of these embodiments, the hinge region comprises an S241P substitution and an L236E substitution.

In other embodiments, an anti-C1s antibody suitable for the present disclosure comprises a heavy chain comprising an amino acid sequence at least about 80%, at least about 85%, at least about 90%, at least about 95%, at least about 96%, at least about 97%, at least about 98%, at least about 99%, or about 100% identical to the amino acid sequence as set forth in SEQ ID NO: XX. In some embodiments, an anti-C1s antibody suitable for the present disclosure comprises a light chain comprising an amino acid sequence at least about 80% at least about 85% at least about 90% at least about 95%, at least about 96%, at least about 97%, at least about 98%, at least about 99%, or about 100% identical to the amino acid sequence as set forth in SEQ ID NO: XX. In other embodiments, an anti-C1s antibody suitable for the present disclosure comprises a heavy chain comprising an amino acid sequence at least about 80%, at least about 85%, at least about 90%, at least about 95%, at least about 96%, at least about 97%, at least about 98%, at least about 99%, or about 100% identical to the amino acid sequence as set forth in SEQ ID NO: XX and a light chain comprising an amino acid sequence at least about 80%, at least about 85%, at least about 90%, at least about 95%, at least about 96%, at least about 97%, at least about 98%, at least about 99%, or about 100% identical to the amino acid sequence as set forth in SEQ ID NO: XX. In some embodiments, an anti-C1s antibody suitable for the present disclosure comprises a heavy chain comprising an amino acid sequence at least about 80%, at least about 85%, at least about 90%, at least about 95%, at least about 96%, at least about 97%, at least about 98%, at least about 99%, or about 100% identical to the amino acid sequence as set forth in SEQ ID NO: XX and a light chain comprising an amino acid sequence at least about 80%, at least about 85%, at least about 90%, at least about 95%, at least about 96%, at least about 97%, at least about 98%, at least about 99%, or about 100% identical to the amino acid sequence as set forth in SEQ ID NO: XX, wherein the anti-C1s antibody comprises the six CDRs of BIVV009.

An anti-C1s antibody suitable for the present disclosure can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than the anti-C1s antibody, etc.

### Compositions

An anti-C1s antibody is generally present in a composition, e.g., a pharmaceutical composition.

A composition comprising an anti-C1s antibody can comprise one or more of a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

In carrying out a method of the present disclosure, an anti-C1s antibody can be administered to an individual using any convenient means capable of resulting in the desired therapeutic effect or diagnostic effect. Thus, the anti-C1s antibody can be incorporated into a variety of formulations for therapeutic administration. More particularly, an anti-C1s antibody can be formulated into pharmaceutical compositions by combination with appropriate, pharmaceutically acceptable carriers, pharmaceutically acceptable diluents, or other pharmaceutically acceptable excipients and can be formulated into preparations in solid, semi-solid, liquid or gaseous forms, such as tablets, capsules, powders, granules, ointments, solutions, suppositories, injections, inhalants and aerosols. In some cases, a pharmaceutical composition comprises an anti-C1s antibody and a pharmaceutically acceptable excipient.

In pharmaceutical dosage forms, an anti-C1s antibody can be administered in the form of their pharmaceutically acceptable salts, or they can also be used alone or in appropriate association, as well as in combination, with other pharmaceutically active compounds. The following methods and excipients are merely exemplary and are in no way limiting.

For oral preparations, an anti-C1s antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An anti-C1s antibody can be formulated into preparations for injection by dissolving, suspending or emulsifying the antibody in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, the pharmaceutical composition of the present disclosure can comprise further agents such as dopamine or psychopharmacologic drugs, depending on the intended use of the pharmaceutical composition.

Pharmaceutical compositions comprising an anti-C1s antibody are prepared by mixing a subject antibody having the desired degree of purity with optional physiologically acceptable carriers, other excipients, stabilizers, surfactants, buffers and/or tonicity agents. Acceptable carriers, other excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

Exemplary antibody concentrations in a pharmaceutical composition suitable for use in a method of the present disclosure can range from about 1 mg/mL to about 200 mg/mL or from about 50 mg/mL to about 200 mg/mL, or from about 150 mg/mL to about 200 mg/mL. In some aspects, the antibody concentration is from about 10 mg/mL to about 60 mg/mL, from about 12 mg/mL to about 58 mg/mL, from about 14 mg/mL to about 56 mg/mL, from about 16 mg/mL to about 54 mg/mL, from about 17 mg/mL to about 52 mg/mL, or from about 18 mg/mL to about 50 mg/mL. In some aspects, the antibody concentration is 18 mg/mL. In some aspects, the antibody concentration is 50 mg/mL.

An aqueous formulation of an anti-C1s antibody can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 nM.

A surfactant can also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark TWEEN 80™) Examples of suitable polyethylene-polypropylene copolymers are those sold under the names PLURONIC® F68 or POLOXAMER 188™. Examples of suitable Polyoxyethylene alkyl ethers are those sold under the trademark BRIJ™. Exemplary concentrations of surfactant can range from about 0.001% to about 1% w/v.

A lyoprotectant can also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some case, a suitable formulation includes an anti-C1s antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a suitable formulation can be a liquid or lyophilized formulation suitable for parenteral administration, and can comprise: about 1 mg/mL to about 200 mg/mL of a subject antibody; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a suitable parenteral formulation is a liquid or lyophilized formulation comprising: about 1 mg/mL to about 200 mg/mL of an anti-C1s antibody; 0.04% Tween 20 w/v; 20 mM L-histidine; and 250 mM sucrose; and has a pH of 5.5.

As another example, a subject parenteral formulation comprises a lyophilized formulation comprising: 1) 15 mg/mL of an anti-C1s antibody; 0.04% Tween 20 w/v; 20 mM L-histidine; and 250 mM sucrose; and has a pH of 5.5; or 2) 75 mg/mL of a subject antibody; 0.04% Tween 20 w/v; 20 mM L-histidine; and 250 mM sucrose; and has a pH of 5.5; or 3) 75 mg/mL of an anti-C1s antibody; 0.02% Tween 20 w/v; 20 mM L-histidine; and 250 mM sucrose; and has a pH of 5.5; or 4) 75 mg/mL of an anti-C1s antibody; 0.04% Tween 20 w/v; 20 mM L-histidine; and 250 mM trehalose; and has a pH of 5.5; or 5) 75 mg/mL of an anti-C1s antibody; 0.02% Tween 20 w/v; 20 mM L-histidine; and 250 mM trehalose; and has a pH of 5.5.

As another example, a suitable parenteral formulation is a liquid formulation comprising: 1) 7.5 mg/mL of an anti-C1s antibody; 0.02% Tween 20 w/v; 120 mM L-histidine; and 250 125 mM sucrose; and has a pH of 5.5; or 2) 37.5 mg/mL of an anti-C1s antibody; 0.02% Tween 20 w/v; 10 mM L-histidine; and 125 mM sucrose; and has a pH of 5.5; or 3) 37.5 mg/mL of an anti-C1s antibody; 0.01% Tween 20 w/v; 10 mM L-histidine; and 125 mM sucrose; and has a pH of 5.5; or 4) 37.5 mg/mL of an anti-C1s antibody; 0.02% Tween 20 w/v; 10 mM L-histidine; 125 mM trehalose; and has a pH of 5.5; or 5) 37.5 mg/mL of an anti-C1s antibody; 0.01% Tween 20 w/v; 10 mM L-histidine; and 125 mM trehalose; and has a pH of 5.5; or 6) 5 mg/mL of an anti-C1s antibody; 0.02% Tween 20 w/v; 20 mM L-histidine; and 250 mM trehalose; and has a pH of 5.5; or 7) 75 mg/mL of an anti-C1s antibody; 0.02% Tween 20 w/v; 20 mM L-histidine; and 250 mM mannitol; and has a pH of 5.5; or 8) 75 mg/mL of an anti-C1s antibody; 0.02% Tween 20 w/v; 20 mM L histidine; and 140 mM sodium chloride; and has a pH of 5.5; or 9) 150 mg/mL of an anti-C1s antibody; 0.02% Tween 20 w/v; 20 mM L-histidine; and 250 mM trehalose; and has a pH of 5.5; or 10) 150 mg/mL of an anti-C1s antibody; 0.02% Tween 20 w/v; 20 mM L-histidine; and 250 mM mannitol; and has a pH of 5.5; or 11) 150 mg/mL of an anti-C1s antibody; 0.02% Tween 20 w/v; 20 mM L-histidine; and 140 mM sodium chloride; and has a pH of 5.5; or 12) 10 mg/mL of an anti-C1s antibody; 0.01% Tween 20 w/v; 20 mM L-histidine; and 40 mM sodium chloride; and has a pH of 5.5.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of a subject antibody adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

The present disclosure provides a method of treating a complement-mediated disease or disorder in an individual, the method comprising administering an anti-C1s antibody to the individual, where the anti-C1s antibody is administered in an effective amount of at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, or at least 10 g.

In some aspects, the anti-C1s antibody is administered in an effective amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some aspects, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some aspects, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

In one aspect, the present disclosure provides a method of treating a complement-mediated disease or disorder in an individual, the method comprising administering an anti-C1s antibody to the individual, where the anti-C1s antibody is administered in an amount of 5.5 g. In some cases, a dose of 5.5 g of the anti-C1s antibody is administered to the individual every other week. In some cases, the method comprises: a) administering 5.5 g of the anti-C1s antibody on Day 1; b) administering 5.5 g of the anti-C1s antibody on Day 8; and c) administering 5.5 g of the anti-C1s antibody every other week following the Day 8 administration. In some cases, a dose of 5.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, a dose of 5.5 g of the anti-C1s antibody is administered to the individual every other week for a period of time of more than 1 year. For example, in some cases, a dose of 5.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In some aspects, the individual for the present method weighs 75 kg or more and the anti-C1s antibody is administered at an effective dose of about 7.5 g. In other aspects, the individual for the present method weighs 75 kg or less and the anti-C1s antibody is administered at an effective dose of about 6.5 g.

In another aspect, the present disclosure also provides a method of treating a complement-mediated disease or disorder in an individual, the method comprising administering an anti-C1s antibody to the individual, where the anti-C1s antibody is administered in an effective dose of about 6.5 g. In some cases, an effective dose of about 6.5 g of the anti-C1s antibody is administered to the individual every other week. In some cases, the method comprises: a) administering an effective dose of about 6.5 g of the anti-C1s antibody on Day 1; b) administering an effective dose of about 6.5 g of the anti-C1s antibody on Day 8; and c) administering an effective dose of about 6.5 g of the anti-C1s antibody every other week following the Day 8 administration. In some cases, an effective dose of about 6.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, an effective dose of about 6.5 g of the anti-C1s antibody is administered to the individual every other week for a period of time of more than 1 year. For example, in some cases, an effective dose of about 6.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In another aspect, the present disclosure also provides a method of treating a complement-mediated disease or disorder in an individual, the method comprising administering an anti-C1s antibody to the individual, where the anti-C1s antibody is administered in an effective dose of about 7.5 g. In some cases, an effective dose of about 7.5 g of the anti-C1s antibody is administered to the individual every other week. In some cases, the method comprises: a) administering an effective dose of about 7.5 g of the anti-C1s antibody on Day 1; b) administering an effective dose of about 7.5 g of the anti-C1s antibody on Day 8; and c) administering an effective dose of about 7.5 g of the anti-C1s antibody every other week following the Day 8 administration. In some cases, an effective dose of about 7.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, an effective dose of about 7.5 g of the anti-C1s antibody is administered to the individual every other week for a period of time of more than 1 year. For example, in some cases, an effective dose of about 7.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

In other aspects, the present disclosure provides a method of treating a complement-mediated disease or disorder in an individual, the method comprising administering an anti-C1s antibody to the individual, where the anti-C1s antibody is administered in an effective dose between about 6.5 g and about 7.5 g. In some cases, an effective dose between about 6.5 g to about 7.5 g of the anti-C1s antibody is administered to the individual every other week. In some cases, the method comprises administering an effective dose between about 6.5 g and about 7.5 g of the anti-C1s antibody on Days 0 and 7 and then every other week thereafter. In some cases, an effective dose between about 6.5 g and 7.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, an effective dose between about 6.5 g and 7.5 g of the anti-C1s antibody is administered to the individual every other week for a period of time of more than 1 year.

The present disclosure provides a method of treating a complement-mediated disease or disorder in a subject in need thereof, the method comprising administering an effective dose of an anti-C1s antibody to the subject, where the serum concentration of the anti-C1s antibody after the administration is at least about 20 μg/mL, at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, or at least about 100 μg/mL. In some aspects of the disclosure, the serum concentration of the anti-C1s antibody after the administration is between about 20 μg/mL and about 100 μg/mL, about 20 μg/mL and about 90 μg/mL, about 20 μg/mL and about 80 μg/mL, about 20 μg/mL and about 70 μg/mL, about 20 μg/mL and about 70 μg/mL, about 20 μg/mL and about 60 μg/mL, about 20 μg/mL and about 50 μg/mL, about 20 μg/mL and about 40 μg/mL, or about 20 μg/mL and about 30 μg/mL. In a particular embodiment, the serum concentration of the anti-C1s antibody after the administration is at least about 20 μg/mL.

The serum concentration of the anti-C1s antibody in the subject can be measured using techniques known in the art. In some aspects, the anti-C1s antibody is measured using a direct binding Enzyme-Linked Immunosorbent Assay (ELISA). In some aspects, the anti-C1s antibody is measured using an indirect ELISA. In some aspects, the anti-C1s antibody is measured using a sandwich ELISA. In some aspects the anti-C1s antibody is measured using a competitive ELISA.

The present disclosure provides a method of treating a complement-mediated disease or disorder in a subject in need thereof, the method comprising administering an effective dose of an anti-C1s antibody to the subject, wherein the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg. In a specific embodiment, the effective dose of the anti-C1s antibody is at least about 60 mg/kg.

In some aspects, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some aspects, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some aspects, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some aspects, the effective dose for the present methods is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

The present disclosure provides a method of treating a complement-mediated disease or disorder in a subject in need thereof, the method comprising administering an effective dose of an anti-C1s antibody to the subject, wherein the anti-C1s antibody is administered at a dosing interval of five days, six days, seven days, eight days, nine days, ten days, eleven days, twelve days, thirteen days, fourteen days, fifteen days, sixteen days, seventeen days, eighteen days, nineteen days, twenty days, twenty one days, twenty two days, twenty three days, twenty four days, twenty five days, twenty six days, twenty seven days, twenty eight days, twenty nine days, thirty days, or thirty one days.

In some aspects, the anti-C1s antibody is administered at a dosing interval of one week, two weeks, three weeks, four weeks, one month, two months, three months, or four months. In some aspects, the anti-C1s antibody increases the number of reticulocytes in the subject's blood after the administration of the anti-C1s antibody.

In some aspects, the anti-C1s antibody is administered as one or more loading doses followed by dosing at dosing intervals. The loading doses can be administered about 7 days apart, about 14 days apart, about 21 days apart, about 28 days apart, about two months apart, about three months apart, or about four months apart. In some aspects, the loading dose for the present disclosure is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg. In some aspects, the loading dose is a different dosage amount than the dose administered at dosing intervals. In some aspects, the loading dose is the same dosage amount as the dose administered at dosing intervals. In one aspect, the anti-C1s antibody is administered as two weekly loading doses of 60 mg/kg followed by doses of 60 mg/kg administered every other week.

The present disclosure provides a method of increasing the number of reticulocytes in the blood of a subject in need thereof, comprising administering to the subject an effective dose of an anti-C1s antibody. In some aspects, the anti-C1s antibody increases the number of reticulocytes in the blood of the subject after the administration at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2.0 fold, at least about 2.1 fold, at least about 2.2 fold, at least about 2.3 fold, at least about 2.4 fold, at least about 2.5 fold, at least about 2.6 fold, at least about 2.7 fold, at least about 2.8 fold, at least about 2.9 fold, at least about 3.0 fold, at least about 4 fold, at least about 5 fold, at least about 6 fold, at least about 7 fold, at least about 8 fold, at least about 9 fold, or at least about 10 fold.

In some aspects, the anti-C1s antibody increases the number of reticulocytes in the blood of the subject within about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 5 weeks, about 6 weeks, about 7 weeks, about 8 weeks, about 9 weeks, about 10 weeks, about 11 weeks, or about 12 weeks of the administration.

The present disclosure provides a method of increasing the number of reticulocytes in the blood of a subject in need thereof, the method comprising administering an effective dose of an anti-C1s antibody to the subject, wherein the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

In some aspects, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some aspects, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some aspects, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some aspects, the effective dose for the present methods is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

The present disclosure also provides a method of increasing the number of reticulocytes in the blood of a subject in need thereof, the method comprising administering an anti-C1s antibody to the individual, where the anti-C1s antibody is administered in an amount of at least about 4 g, at least about 4.5 g, at least about 5 g, at least about 5.5 g, at least about 6 g, at least about 6.5 g, at least about 7 g, at least about 7.5 g, at least about 8 g, at least about 8.5 g, at least about 9 g, at least about 9.5 g, or at least about 10 g.

In some aspects, the anti-C1s antibody is administered in an amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some aspects, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some aspects, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

The present disclosure provides a method of increasing the level of hemoglobin in a subject in need thereof, comprising administering to the subject an effective dose of an anti-C1s antibody. In some aspects, the anti-C1s antibody increases the level of hemoglobin in the subject after the administration at least 1.0 g/dL, 1.1 g/dL, 1.2 g/dL, 1.3 g/dL, 1.4 g/dL, 1.5 g/dL, 1.6 g/dL, 1.7 g/dL, 1.8 g/dL, 1.9 g/dL, 2.0 g/dL, 2.1 g/dL, 2.2 g/dL, 2.3 g/dL, 2.4 g/dL, 2.5 g/dL, 2.6 g/dL, 2.7 g/dL, 2.8 g/dL, 2.9 g/dL, 3.0 g/dL, 3.1 g/dL, 3.2 g/dL, 3.3 g/dL, 3.4 g/dL, 3.5 g/dL, 3.6 g/dL, 3.7 g/dL, 3.8 g/dL, 3.9 g/dL, 4.0 g/dL, 4.1 g/dL, 4.2 g/dL, 4.3 g/dL, 4.4 g/dL, or 4.5 g/dL.

In some aspects, the anti-C1s antibody increases the total level of hemoglobin in the subject after the administration to at least 10.0 g/dL, at least 10.1 g/dL, at least 10.2 g/dL, at least 10.3 g/dL, at least 10.4 g/dL, at least 10.5 g/dL, at least 10.6 g/dL, at least 10.7 g/dL, at least 10.8 g/dL, at least 10.9 g/dL, at least 11.0 g/dL, at least 11.1 g/dL, at least 11.2 g/dL, at least 11.3 g/dL, at least 11.4 g/dL, at least 11.5 g/dL, at least 11.6 g/dL, at least 11.7 g/dL, at least 11.8 g/dL, at least 11.9 g/dL, at least 12.0 g/dL, at least 12.1 g/dL, at least 12.2 g/dL, at least 12.3 g/dL, at least 12.4 g/dL, at least 12.5 g/dL, at least 12.6 g/dL, at least 12.7 g/dL, at least 12.8 g/dL, at least 12.9 g/dL, at least 13.0 g/dL, at least 13.1 g/dL, at least 13.2 g/dL, at least 13.3 g/dL, at least 13.4 g/dL, at least 13.5 g/dL, at least 13.6 g/dL, at least 13.7 g/dL, at least 13.8 g/dL, at least 13.9 g/dL, at least 14.0 g/dL, at least 14.1 g/dL, at least 14.2 g/dL, at least 14.3 g/dL, at least 14.4 g/dL, at least 14.5 g/dL, at least 14.6 g/dL, at least 14.7 g/dL, at least 14.8 g/dL, at least 14.9 g/dL, at least 15.0 g/dL, at least 15.1 g/dL, at least 15.2 g/dL, at least 15.3 g/dL, at least 15.4 g/dL, at least 15.5 g/dL, at least 15.6 g/dL, at least 15.7 g/dL, at least 15.8 g/dL, at least 15.9 g/dL, at least 16.0 g/dL, at least 16.1 g/dL, at least 16.2 g/dL, at least 16.3 g/dL, at least 16.4 g/dL, at least 16.5 g/dL, at least 16.6 g/dL, at least 16.7 g/dL, at least 16.8 g/dL, at least 16.9 g/dL, at least 17.0 g/dL, at least 17.1 g/dL, at least 17.2 g/dL, at least 17.3 g/dL, at least 17.4 g/dL, at least 17.5 g/dL, at least 17.6 g/dL, at least 17.7 g/dL, at least 17.8 g/dL, at least 17.9 g/dL, or at least 18.0 g/dL.

In some aspects, the anti-C1s antibody increases the level of hemoglobin in the subject within about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 days, about 11 days, about 12 days about 13 days, about 14 days, about 2 weeks, about 3 weeks, about 4 weeks, about 5 weeks, about 6 weeks, about 7 weeks, about 8 weeks, about 9 weeks, about 10 weeks, about 11 weeks, or about 12 weeks of the administration.

In a particular aspect, the present disclosure provides a method of increasing the level of hemoglobin in a subject in need thereof, e.g., blood, comprising administering to the subject an effective dose of an anti-C1s antibody, wherein the level of hemoglobin in the subject, e.g., blood, is increased at least by 1.6 g/dL within seven days from the administration. In another aspect, the level of hemoglobin in the subject is increased up to 3.9 g/dL within six weeks from the administration.

The present disclosure provides a method of increasing the level of hemoglobin in a subject in need thereof, the method comprising administering an effective dose of an anti-C1s antibody to the subject, wherein the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

In some aspects, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some aspects, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some aspects, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some aspects, the effective dose is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

The present disclosure also provides a method of increasing the level of hemoglobin in a subject in need thereof, the method comprising administering an anti-C1s antibody to the individual, where the anti-C1s antibody is administered in an amount of at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, or at least 10 g.

In some aspects, the anti-C1s antibody is administered in an amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some aspects, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some aspects, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

The present disclosure provides a method of decreasing the percentage of C3d positive erythrocytes in the blood of a subject in need thereof, comprising administering to the subject an effective dose of an anti-C1s antibody. In some aspects, the anti-C1s antibody decreases the percentage of C3d positive erythrocytes in the blood of the subject at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. compared to the percentage of C3d positive erythrocytes in the blood of the subject prior to the administration.

In some aspects, the anti-C1s antibody decreases the percentage of C3d positive erythrocytes in the blood of the subject within about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 days, about 11 days, about 12 days about 13 days, about 14 days, about 2 weeks, about 3 weeks, about 4 weeks, about 5 weeks, about 6 weeks, about 7 weeks, about 8 weeks, about 9 weeks, about 10 weeks, about 11 weeks, or about 12 weeks of the administration.

The present disclosure provides a method of decreasing the percentage of C3d positive erythrocytes in the administration of a subject in need thereof, the method comprising administering an effective dose of an anti-C1s antibody to the subject, wherein the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

In some aspects, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some aspects, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some aspects, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some aspects, the effective dose is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

The present disclosure also provides a method of decreasing the percentage of C3d positive erythrocytes in the blood of a subject in need thereof, the method comprising administering an anti-C1s antibody to the individual, where the anti-C1s antibody is administered in an amount of at least about 4 g, at least about 4.5 g, at least about 5 g, at least about 5.5 g, at least about 6 g, at least about 6.5 g, at least about 7 g, at least about 7.5 g, at least about 8 g, at least about 8.5 g, at least about 9 g, at least about 9.5 g, or at least about 10 g.

In some aspects, the anti-C1s antibody is administered in an amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some aspects, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some aspects, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

The present disclosure provides a method of decreasing the level of bilirubin in a subject in need thereof, e.g., blood, comprising administering to the subject an effective dose of an anti-C1s antibody. In some aspects, the anti-C1s antibody decreases the level of bilirubin in the subject to be lower than 2.5 mg/dL, 2.4 mg/dL, 2.3 mg/dL, 2.2 mg/dL, 2.1 mg/dL, 2.0 mg/dL, 1.9 mg/dL, 1.8 mg/dL, 1.7 mg/dL, 1.6 mg/dL, 1.5 mg/dL, 1.4 mg/dL, 1.3 mg/dL, 1.2 mg/dL, 1.1 mg/dL, 1.0 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL.

In some aspects, the anti-C1s antibody decreases the level of bilirubin in the subject, e.g., blood, within about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 days, about 11 days, about 12 days about 13 days, about 14 days, about 2 weeks, about 3 weeks, about 4 weeks, about 5 weeks, about 6 weeks, about 7 weeks, about 8 weeks, about 9 weeks, about 10 weeks, about 11 weeks, or about 12 weeks of the administration.

The present disclosure provides a method of decreasing the level of bilirubin in a subject in need thereof, e.g., blood, the method comprising administering an effective dose of an anti-C1s antibody to the subject, wherein the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

In some aspects, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some aspects, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some aspects, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some aspects, the effective dose for the present methods is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

The present disclosure also provides a method of decreasing the level of bilirubin in a subject in need thereof, e.g., blood, the method comprising administering an anti-C1s antibody to the individual, where the anti-C1s antibody is administered in an effective amount of at least about 4 g, at least about 4.5 g, at least about 5 g, at least about 5.5 g, at least about 6 g, at least about 6.5 g, at least about 7 g, at least about 7.5 g, at least about 8 g, at least about 8.5 g, at least about 9 g, at least about 9.5 g, or at least about 10 g.

In some aspects, the anti-C1s antibody is administered in an effective amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some aspects, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some aspects, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

The present disclosure provides a method inhibiting cleavage of complement component C4 in an individual, the method comprising administering an anti-C1s antibody to the individual, where the anti-C1s antibody is administered in an effective amount of about 5.5 g. In some cases, an effective dose of about 5.5 g of the anti-C1s antibody is administered to the individual once every other week. In some cases, the method comprises: a) administering an effective amount of about 5.5 g of the anti-C1s antibody on Day 1; b) administering an effective amount of about 5.5 g of the anti-C1s antibody on Day 8; and c) administering an effective amount of about 5.5 g of the anti-C1s antibody every other week following the Day 8 administration. In some cases, an effective amount of about 5.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, an effective amount of about 5.5 g of the anti-C1s antibody is administered to the individual every other week for a period of time of more than 1 year. For example, in some cases, an effective amount of about 5.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

The present disclosure provides a method inhibiting cleavage of complement component C4 in an individual, the method comprising administering an anti-C1s antibody to the individual, where the anti-C1s antibody is administered in an effective amount of about 6.5 g. In some cases, a dose of an effective amount of about 6.5 g of the anti-C1s antibody is administered to the individual once every other week. In some cases, the method comprises: a) administering an effective amount of about 6.5 g of the anti-C1s antibody on Day 1; b) administering an effective amount of about 6.5 g of the anti-C1s antibody on Day 8; and c) administering an effective amount of about 6.5 g of the anti-C1s antibody every other week following the Day 8 administration. In some cases, an effective amount of about 6.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, an effective amount of about 6.5 g of the anti-C1s antibody is administered to the individual every other week for a period of time of more than 1 year. For example, in some cases, an effective amount of about 6.5 g of the anti-C1s antibody is administered to the individual every other week for a period of time from 1 year to 50 years, e.g., from 1 year to 2 years, from 2 years to 5 years, from 5 years to 10 years, from 10 years to 20 years, from 20 years to 30 years, from 30 years to 40 years, or from 40 years to 50 years.

The present disclosure provides a method inhibiting cleavage of complement component C4 in an individual, the method comprising administering an anti-C1s antibody to the individual, where the anti-C1s antibody is administered in an effective amount of about 7.5 g. In some cases, an effective amount of about 7.5 g of the anti-C1s antibody is administered to the individual once every other week. In some cases, the method comprises: a) administering an effective amount of about 7.5 g of the anti-C1s antibody on Day 1; b) administering an effective amount of about 7.5 g of the anti-C1s antibody on Day 8; and c) administering an effective amount of about 7.5 g of the anti-C1s antibody every other week following the Day 8 administration. In some cases, an effective amount of about 7.5 g of the anti-C1s antibody is administered to the individual every other week for a period of time from about 4 weeks to 1 year, e.g., from about 4 weeks to about 8 weeks, from about 2 months to about 6 months, or from about 6 months to 1 year. In some cases, an effective amount of about 7.5 g of the anti-C1s antibody is administered to the individual every other week for a period of time of more than 1 year. For example, in some cases, an effective amount of about 7.5 g of the anti-C1s antibody is administered to the individual every other week for a period of time from 1 year to 50 years,e.g., from 1 year to 2 years, from 2 years to 5 years, from 5 years to 10 years, from 10 years to 20 years, from 20 years to 30 years, from 30 years to 40 years, or from 40 years to 50 years. Route of administration

An anti-C1s antibody is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intrathecal, intracranial, subcutaneous, intradermal, topical, intravenous, intraperitoneal, intraarterial (e.g., via the carotid artery), spinal or brain delivery, rectal, nasal, oral, and other enteral and parenteral routes of administration. Routes of administration can be combined, if desired, or adjusted depending upon the antibody and/or the desired effect. An anti-C1s antibody composition can be administered in a single dose or in multiple doses. In some cases, an anti-C1s antibody is administered orally. In some cases, an anti-C1s antibody is administered subcutaneously. In some cases, an anti-C1s antibody is administered intramuscularly. In some cases, an anti-C1s antibody is administered intravenously.

An anti-C1s antibody can be administered to a host using any available conventional methods and routes suitable for delivery of conventional drugs, including systemic or localized routes. In general, routes of administration contemplated by the disclosure include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

By “treatment” is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as a complement-mediated disease or disorder. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

In some cases, an anti-C1s antibody is administered by injection and/or delivery, e.g., to a site in a brain artery or directly into brain tissue. An anti-C1s antibody can also be administered directly to a target site e.g., by biolistic delivery to the target site.

A variety of hosts (wherein the term “host” is used interchangeably herein with the terms “subject,” “individual,” and “patient”) are treatable according to the subject methods. Generally such hosts are “mammals” or “mammalian,” where these terms are used broadly to describe organisms which are within the class mammalia, including the orders carnivore (e.g., cats), herbivores (e.g., cattle, horses, and sheep), omnivores (e.g., dogs, goats, and pigs), rodentia (e.g., mice, guinea pigs, and rats), and primates (e.g., humans, chimpanzees, and monkeys). In some embodiments, the host is an individual that has a complement system, such as a mammal, fish, or invertebrate. In some cases, the host is a complement system-containing mammal, fish, or invertebrate companion animal, agricultural animal, work animal, zoo animal, or lab animal. In some cases, the individual is human.

### Complement-Mediated Diseases and Disorders

In some cases, a complement-mediated disease or disorder is characterized by the presence in a cell, a tissue, or a fluid of an elevated (higher than normal) amount of C1s or of an elevated level of complement C1s activity. For example, in some cases, a complement-mediated disease or disorder is characterized by the presence in brain tissue and/or cerebrospinal fluid of an elevated amount and/or an elevated activity of Cis. A “higher than normal” amount of C1s in a cell, a tissue, or a fluid indicates that the amount of C1s in the cell, tissue or fluid is higher than a normal, control level, e.g., higher than a normal, control level for an individual or population of individuals of the same age group. A “higher than normal” level of C1s activity in a cell, a tissue, or a fluid indicates that the proteolytic cleavage effected by C1s in the cell, tissue or fluid is higher than a normal, control level, e.g., higher than a normal, control level for an individual or population of individuals of the same age group. In some cases, an individual having a complement-mediated disease or disorder exhibits one or more additional symptoms of such a disease or disorder.

In other cases, a complement-mediated disease or disorder is characterized by the presence in a cell, a tissue, or a fluid of a lower than normal amount of C1s or of a lower level of complement C1s activity. For example, in some cases, a complement-mediated disease or disorder is characterized by the presence in brain tissue and/or cerebrospinal fluid of a lower amount and/or a lower activity of C1s. A “lower than normal” amount of C1s in a cell, a tissue, or a fluid indicates that the amount of C1s in the cell, tissue or fluid is lower than a normal, control level, e.g., lower than a normal, control level for an individual or population of individuals of the same age group. A “lower than normal” level of C1s activity in a cell, a tissue, or a fluid indicates that the proteolytic cleavage effected by C1s in the cell, tissue or fluid is lower than a normal, control level, e.g., lower than a normal, control level for an individual or population of individuals of the same age group. In some cases, an individual having a complement-mediated disease or disorder exhibits one or more additional symptoms of such a disease or disorder.

A complement-mediated disease or disorder is a disease or disorder in which the amount or activity of complement C1s is such as to cause disease or disorder in an individual. In some embodiments, the complement-mediated disease or disorder is selected from the group consisting of autoimmune disease, cancer, hematological disease, infectious disease, inflammatory disease, ischemia-reperfusion injury, neurodegenerative disease, neurodegenerative disorder, ocular disease, renal disease, transplant rejection, vascular disease, and vasculitis disease. In some cases, the complement-mediated disease or disorder is an autoimmune disease. In some cases, the complement-mediated disease or disorder is cancer. In some cases, the complement-mediated disease or disorder is an infectious disease. In some cases, the complement-mediated disease or disorder is an inflammatory disease. In some cases, the complement-mediated disease or disorder is a hematological disease. In some cases, the complement-mediated disease or disorder is an ischemia-reperfusion injury. In some cases, the complement-mediated disease or disorder is ocular disease. In some cases, the complement-mediated disease or disorder is a renal disease. In some cases, the complement-mediated disease or disorder is transplant rejection. In some cases, the complement-mediated disease or disorder is antibody-mediated transplant rejection. In some cases, the complement-mediated disease or disorder is a vascular disease. In some cases, the complement-mediated disease or disorder is a vasculitis disorder. In some cases, the complement-mediated disease or disorder is a neurodegenerative disease or disorder. In some cases, the complement-mediated disease is a neurodegenerative disease. In some cases, the complement-mediated disorder is a neurodegenerative disorder. In some cases, the complement-mediated disease or disorder is a tauopathy.

Examples of a complement-mediated disease or disorder include, but are not limited to, age-related macular degeneration, Alzheimer's disease, amyotrophic lateral sclerosis, anaphylaxis, argyrophilic grain dementia, arthritis (e.g., rheumatoid arthritis), asthma, atherosclerosis, atypical hemolytic uremic syndrome, autoimmune diseases, Barraquer-Simons syndrome, Behçet's disease, British type amyloid angiopathy, bullous pemphigoid, Buerger's disease, C1q nephropathy, chronic inflammatory demyelinating polyneuropathy, cancer, catastrophic antiphospholipid syndrome, cerebral amyloid angiopathy, cold agglutinin disease, (including primary cold agglutinin disease and secondary cold agglutinin disease), corticobasal degeneration, Creutzfeldt-Jakob disease, Crohn's disease, cryoglobulinemic vasculitis, dementia pugilistica, dementia with Lewy Bodies (DLB), diffuse neurofibrillary tangles with calcification, Discoid lupus erythematosus, Down's syndrome, focal segmental glomerulosclerosis, formal thought disorder, frontotemporal dementia (FTD), frontotemporal dementia with parkinsonism linked to chromosome 17, frontotemporal lobar degeneration, Gerstmann-Straussler-Scheinker disease, Guillain-Barré syndrome, Hallervorden-Spatz disease, hemolytic-uremic syndrome, hereditary angioedema, hypophosphastasis, idiopathic pneumonia syndrome, immune complex diseases, inclusion body myositis, infectious disease (e.g., disease caused by bacterial (e.g., Neisseria meningitidis or Streptococcus) viral (e.g., human immunodeficiency virus (HIV)), or other infectious agents), inflammatory disease, ischemia/reperfusion injury, mild cognitive impairment, immunothrombocytopenic purpura (ITP), molybdenum cofactor deficiency (MoCD) type A, membranoproliferative glomerulonephritis (MPGN) I, membranoproliferative glomerulonephritis (MPGN) II (dense deposit disease), membranous nephritis, multi-infarct dementia, lupus (e.g., systemic lupus erythematosus (SLE)), glomerulonephritis, Kawasaki disease, mucous membrane pemphigoid, cicatricial pemphigoid, multifocal motor neuropathy, multiple sclerosis, multiple system atrophy, myasthenia gravis, myocardial infarction, myotonic dystrophy, neuromyelitis optica, Niemann-Pick disease type C, non-Guamanian motor neuron disease with neurofibrillary tangles, Parkinson's disease, Parkinson's disease with dementia, paroxysmal nocturnal hemoglobinuria, Pemphigus vulgaris, Pick's disease, postencephalitic parkinsonism, polymyositis, prion protein cerebral amyloid angiopathy, progressive subcortical gliosis, progressive supranuclear palsy, psoriasis, sepsis, Shiga-toxin E. coli (STEC)-HuS, spinal muscular atrophy, stroke, subacute sclerosing panencephalitis, Tangle only dementia, transplant rejection, vasculitis (e.g., ANCA associated vasculitis), Wegner's granulomatosis, sickle cell disease, cryoglobulinemia, mixed cryoglobulinemia, essential mixed cryoglobulinemia, Type II mixed cryoglobulinemia, Type III mixed cryoglobulinemia, nephritis, drug-induced thrombocytopenia, lupus nephritis, bullous pemphigoid, Epidermolysis bullosa acquisita, delayed hemolytic transfusion reaction, hypocomplementemic urticarial vasculitis syndrome, pseudophakic bullous keratopathy, and platelet refractoriness.

In some cases, the present method includes treatment of primary CAgD in a subject in need thereof comprising administering an effective dose between about 6.5 g and about 7.5 g, e.g., about 6.5 g for subjects with less than 75 kg of bodyweight and 7.5 g for subject with more than 75 kg of bodyweight, of an anti-C1s antibody, e.g., BIVV009. In some embodiments, the present methods have no limitation of use associated with anemia severity, transfusion history, or prior treatment experience. In some embodiments, there is no REMS requirement prior to dosing; vaccinate patients according to local guidelines prior to treatment initiation to reduce risk of serious infection. In some embodiments, the dose is administered as intravenous infusion over 1 hour on Day 0, Day 7, and every 14 days±2 days thereafter starting on Day 21. Intravenous infusion can take place within clinic or home setting. As a result of the treatment, the anti-C1s antibody can improve anemia and associated clinical symptoms, eliminate transfusion, prevent hemolysis, rapid onset of action, improve fatigue and quality of life, and/or any combination thereof. In other embodiments, the treatment shows no drug related serious or severe adverse events; no discontinuations due to adverse events, no serious infections; no REMS requirement, most commonly reported adverse events were similar to placebo, or any combination thereof. In other embodiments, as a result of the treatment, the anti-C1s antibody prevents chronic hemolysis, resulting in improvement in anemia, elimination of transfusion, improvement of quality of life, and ultimately reduction of risk of life threatening thromboembolic events, morbidity, and mortality, and reduced healthcare utilization.

In some cases, the complement-mediated disease or disorder is bullous pemphigoid. In some cases, the complement-mediated disease or disorder is antibody-mediated rejection of organ transplant. In some cases, the complement-mediated disease or disorder is cold agglutinin disease. In some cases, the complement-mediated disease or disorder is warm autoimmune hemolytic anemia. In some cases, the complement-mediated disease or disorder antibody-mediated transplant rejection. In some cases, the complement-mediated disease or disorder is immunothrombocytopenic purpura. In some cases, the complement-mediated disease or disorder is neuromyelitis optica.

In some cases, the complement-mediated disease or disorder is multifocal motor neuropathy (MMN). In some cases, the complement-mediated disease or disorder is myasthenia gravis. In some cases, the complement-mediated disease or disorder is chronic inflammatory demyelinating polyneuropathy. In some cases, the complement-mediated disease or disorder is lupus nephritis. In some cases, the complement-mediated disease or disorder is mucous membrane pemphigoid. In some cases, the complement-mediated disease or disorder is cicatricial pemphigoid. In some cases, the complement-mediated disease or disorder is ocular pemphigoid. In some cases, the complement-mediated disease or disorder is antineutrophil cytoplasmic autoantibody (ANCA) associated vasculitis.

In other embodiments, the complement-mediated disease or disorder is an autoantibody mediated peripheral neuropathy including, but not limited to, Guillain-Barré syndrome, Myasthenia Gravis, acute inflammatory demyelinating polyneuropathy (AIDP), chronic inflammatory demyelinating polyneuropathy (CIDP), acute motor axonal neuropathy (AMAN), acute motor and sensory axonal neuropathy (AMSAN), pharyngeal-cervical brachial variant, Miller Fisher syndrome, or any combination thereof. In some embodiments, the complement-mediated disease or disorder is Guillain-Barré syndrome, which presents as rapid-onset muscle weakness, beginning in the feet and hands that spreads to the arms and upper body. During the acute phase, it can be fatal as respiratory failure can occur, and other autonomic functions (such as heart rate) can be affected. ˜7.5% of all cases are fatal. Incidence: 1-2/100,000.

In other embodiments, the complement-mediated disease or disorder is Myasthenia Gravis, which exhibits weakness, fatigue that becomes progressively worse during periods of physical activity, generally starts with ocular weakness; progressing to a more severe form, characterized by weakness in the extremities and performing basic life functions (chewing, swallowing, breathing). In a myasthenic crisis, respiratory paralysis occurs, necessitating assisted ventilation to sustain life.

In other embodiments, the complement-mediated disease or disorder is multifocal motor neuropathy (MMN), which is an inflammatory autoimmune disease of the lower nervous system. MMN is a pure motor neuropathy, which has the mean age onset of 40 years. MMN is characterized by: slowly progressive, asymmetric distal limb weakness; conduction block (CB), often affecting ulnar, median, radial or tibial nerves; and/or atrophic muscles. Other clinical features include muscle cramps, fasciculations, and an increase of weakness in cold conditions. GM1-specific IgM antibodies are present in the serum of half of all patients, titers of which correlate with their in vitro complement-activating capacity and disease severity. Intravenous immunoglobulin (IVIg) is effective in MMN. Nevertheless, patients still undergo slowly progressive axonal degeneration and muscle weakness that cannot be fully prevented with chronic IVIg therapy.

In other embodiments, a complement-mediated disease or disorder useful for treatment is neuromyelitis optica (NMO). NMO is caused by anti-Aquaporin-4 IgG autoantibody (NMO-IgG) which activates complement and kills astrocytes resulting in death of oligodendrocytes that myelinate the optic nerve and spinal cord. Vision loss and paralysis occur following attacks.

In other embodiments, a complement-mediated disease or disorder useful for treatment is systemic lupus erythematosus (SLE). Systemic lupus erythematosus (SLE) is an autoimmune disease that affects 0.04% of the population of developed countries. SLE is believed to arise as a result of an impairment in the body's waste disposal system, in which complement plays a key role. In humans, congenital deficiencies of the complement proteins in the C1 complex as well as C2 and C4 are associated with an increased risk of developing SLE. However, a substantial number of patients with SLE develop hypocomplementemia with depletion of C1q and other components of the classical pathway: e.g., complement deposition on RBCs and/or C1q deposition in affected tissues.

In other embodiment, a complement-mediated disease or disorder useful for treatment is lupus nephritis (LN). LN is the renal manifestation of SLE that occurs in 25-50% of patients and is the primary cause of morbidity and mortality. C1q antibodies are closely associated with renal involvement, and are highly predictive of and present during flares. Active LN is rarely observed in the absence of C1q Abs. Multiple studies have shown a negative correlation with C1q Ab titers and serum C1q, and a positive correlation with C1q deposition in the glomeruli in patients with LN.

In some embodiments, a complement-mediated disease or disorder useful for treatment is membranoproliferative glomerulonephritis (type I) (Mixed Cryoglobulinemia). Mixed Cryoglobulinemia is a systemic vasculitis mediated by immune complexes (IC). It appears most often in the context of chronic infections (HCV —80% of MC cases). Clinically, cryoglobulinemia manifests itself with symptoms like weakness and arthralgias and variable cutaneous and visceral organ involvement. Steroids suppress inflammation with success in some patients, but additional plasmapheresis to remove circulating cryoglobulins and immunosuppressive treatment to inhibit the formation of new cryoglobulins are often necessary.

In some cases, the method of the present disclosure comprises administering an effective dose between about 6.5 g and about 7.5 g of an anti-C1s antibody, e.g., BIVV009, to a subject having bullous pemphigoid. In some cases, the method of the present disclosure comprises administering an effective dose between about 6.5 g and about 7.5 g of an anti-C1s antibody, e.g., BIVV009, to a subject having antibody-mediated rejection of organ transplant. In some cases, the method of the present disclosure comprises administering an effective dose between about 6.5 g and about 7.5 g of an anti-C1s antibody, e.g., BIVV009, to a subject having cold agglutinin disease. In some cases, the method of the present disclosure comprises administering an effective dose between about 6.5 g and about 7.5 g of an anti-C1s antibody, e.g., BIVV009, to a subject having warm autoimmune hemolytic anemia. In some cases, the method of the present disclosure comprises administering an effective dose between about 6.5 g and about 7.5 g of an anti-C1s antibody, e.g., BIVV009, to a subject having immunothrombocytopenic purpura. In some cases, the method of the present disclosure comprises administering an effective dose between about 6.5 g and about 7.5 g of an anti-C1s antibody, e.g., BIVV009, to a subject having neuromyelitis optica.

In some cases, the method of the present disclosure comprises administering an effective dose between about 6.5 g and about 7.5 g of an anti-C1s antibody, e.g., BIVV009, to a subject having multifocal motor neuropathy (MMN). In some cases, the method of the present disclosure comprises administering an effective dose between about 6.5 g and about 7.5 g of an anti-C1s antibody, e.g., BIVV009, to a subject having myasthenia gravis. In some cases, the method of the present disclosure comprises administering an effective dose between about 6.5 g and about 7.5 g of an anti-C1s antibody, e.g., BIVV009, to a subject having chronic inflammatory demyelinating polyneuropathy. In some cases, the method of the present disclosure comprises administering an effective dose between about 6.5 g and about 7.5 g of an anti-C1s antibody, e.g., BIVV009, to a subject having lupus nephritis. In some cases, the method of the present disclosure comprises administering an effective dose between about 6.5 g and about 7.5 g of an anti-C1s antibody, e.g., BIVV009, to a subject having mucous membrane pemphigoid. In some cases, the method of the present disclosure comprises administering an effective dose between about 6.5 g and about 7.5 g of an anti-C1s antibody, e.g., BIVV009, to a subject having cicatricial pemphigoid. In some cases, the method of the present disclosure comprises administering an effective dose between about 6.5 g and about 7.5 g of an anti-C1s antibody, e.g., BIVV009, to a subject having ocular pemphigoid. In some cases, the method of the present disclosure comprises administering an effective dose between about 6.5 g and about 7.5 g of an anti-C1s antibody, e.g., BIVV009, to a subject having antineutrophil cytoplasmic autoantibody (ANCA) associated vasculitis.

In some embodiments, administering an anti-C1s antibody of the present disclosure results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (l) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) a reduction in thrombus formation; (x) a reduction in antibody mediated complement activation; (y) a reduction in autoantibody mediated complement activation; (z) an improvement in anemia; (aa) reduction of demyelination; (ab) reduction of eosinophilia; (ac) a reduction of C3 deposition on red blood cells (e.g., a reduction of deposition of C3b, iC3b, etc., onto RBCs); and (ad) a reduction in C3 deposition on platelets (e.g., a reduction of deposition of C3b, iC3b, etc., onto platelets); and (ae) a reduction of anaphylatoxin toxin production; (af) a reduction in autoantibody mediated blister formation; (ag) a reduction in autoantibody induced pruritus; (ah) a reduction in autoantibody induced erythematosus; (ai) a reduction in autoantibody mediated skin erosion; (aj) a reduction in red blood cell destruction due to transfusion reactions; (ak) a reduction in red blood cell lysis due to alloantibodies; (al) a reduction in hemolysis due to transfusion reactions; (am) a reduction in allo-antibody mediated platelet lysis; (an) a reduction in platelet lysis due to transfusion reactions; (ao) a reduction in mast cell activation; (ap) a reduction in mast cell histamine release; (aq) a reduction in vascular permeability; (ar) a reduction in edema; (as) a reduction in complement deposition on transplant graft endothelium; (at) a reduction of anaphylatoxin generation in transplant graft endothelium; (au) a reduction in the separation of the dermal-epidermal junction; (av) a reduction in the generation of anaphylatoxins in the dermal-epidermal junction; (aw) a reduction in alloantibody mediated complement activation in transplant graft endothelium; (ax) a reduction in antibody mediated loss of the neuromuscular junction; (ay) a reduction in complement activation at the neuromuscular junction; (az) a reduction in anaphylatoxin generation at the neuromuscular junction; (ba) a reduction in complement deposition at the neuromuscular junction; (bb) a reduction in paralysis; (bc) a reduction in numbness; (bd) increased bladder control; (be) increased bowel control; (bf) a reduction in mortality associated with autoantibodies; (bg) a reduction in morbidity associated with autoantibodies; and (bh) a reduction in conduction block.

In some cases, an anti-C1s antibody, when administered in a dose of 5.5 g, and when administered in one or more doses as monotherapy or in combination therapy to an individual having a complement-mediated disease or disorder, is effective to achieve and maintain a serum concentration of anti-C1s antibody of at least 100 μg/ml. In some cases, an anti-C1s antibody, when administered in a dose of 6.5 g, and when administered in one or more doses as monotherapy or in combination therapy to an individual having a complement-mediated disease or disorder, is effective to achieve and maintain a serum concentration of anti-C1s antibody of at least 100 μg/ml. In some cases, an anti-C1s antibody, when administered in a dose of 7.5 g, and when administered in one or more doses as monotherapy or in combination therapy to an individual having a complement-mediated disease or disorder, is effective to achieve and maintain a serum concentration of anti-C1s antibody of at least 100 μg/ml.

In some cases, an anti-C1s antibody, when administered in a dose of 5.5 g, and when administered in one or more doses as monotherapy or in combination therapy to an individual having a complement-mediated disease or disorder, is sufficient to inhibit complement classical pathway (CP) by at least 50%, at least 60%, at least 70%, at least 80%, or at least 90%. In some case, an anti-C1s antibody, when administered in a dose of 5.5 g, and when administered in one or more doses as monotherapy or in combination therapy to an individual having a complement-mediated disease or disorder, is effective to inhibit CP by 90%. In some cases, an anti-C1s antibody, when administered in a dose of 6.5 g, and when administered in one or more doses as monotherapy or in combination therapy to an individual having a complement-mediated disease or disorder, is effective to inhibit complement classical pathway (CP) by at least 50%, at least 60%, at least 70%, at least 80%, or at least 90%. In some case, an anti-C1s antibody, when administered in a dose of 6.5 g, and when administered in one or more doses as monotherapy or in combination therapy to an individual having a complement-mediated disease or disorder, is effective to inhibit CP by 90%. In some cases, an anti-C1s antibody, when administered in a dose of 7.5 g, and when administered in one or more doses as monotherapy or in combination therapy to an individual having a complement-mediated disease or disorder, is effective to inhibit complement classical pathway (CP) by at least 50%, at least 60%, at least 70%, at least 80%, or at least 90%. In some case, an anti-C1s antibody, when administered in a dose of 7.5 g, and when administered in one or more doses as monotherapy or in combination therapy to an individual having a complement-mediated disease or disorder, is effective to inhibit CP by 90%.

In some cases, an anti-C1s antibody, when administered in a dose of 5.5 g, and when administered in one or more doses as monotherapy or in combination therapy to an individual having a complement-mediated disease or disorder, is effective to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (l) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; (x) blister formation; (y) pruritus; (z) skin rash; (ab) skin erosions; (ac) petechiae; (ad) bleeding time; (ae) conduction block; compared to the level or degree of the outcome in the individual before treatment with the anti-C1s antibody.

In some cases, an anti-C1s antibody, when administered in a dose of 6.5 g, and when administered in one or more doses as monotherapy or in combination therapy to an individual having a complement-mediated disease or disorder, is effective to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (l) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; (x) blister formation; (y) pruritus; (z) skin rash; (ab) skin erosions; (ac) petechiae; (ad) bleeding time; (ae) conduction block; compared to the level or degree of the outcome in the individual before treatment with the anti-C1s antibody.

In some cases, an anti-C1s antibody, when administered in a dose of 7.5 g, and when administered in one or more doses as monotherapy or in combination therapy to an individual having a complement-mediated disease or disorder, is effective to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (l) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; (x) blister formation; (y) pruritus; (z) skin rash; (ab) skin erosions; (ac) petechiae; (ad) bleeding time; (ae) conduction block; compared to the level or degree of the outcome in the individual before treatment with the anti-C1s antibody.

In some cases, an anti-C1s antibody, when administered in a dose of 5.5 g, and when administered in one or more doses as monotherapy or in combination therapy to an individual having a complement-mediated disease or disorder, is effective to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation (reduction of thrombus formation); f) clotting (reduction of clotting); g) kidney function; h) hematocrit (red blood cell count); i) pruritis; j) blister formation; k) skin rash; l) petechiae; m) platelet count; n) bleeding time; o) conduction block; and p) inflammation (reduction of inflammation), compared to the level or degree of the outcome in the individual before treatment with the anti-C1s antibody.

In some cases, an anti-C1s antibody, when administered in a dose of 6.5 g, and when administered in one or more doses as monotherapy or in combination therapy to an individual having a complement-mediated disease or disorder, is effective to achieve an improvement of at least about 10% at least about 15%, at least about 20% at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation (reduction of thrombus formation); f) clotting (reduction of clotting); g) kidney function; h) hematocrit (red blood cell count); i) pruritis; j) blister formation; k) skin rash; l) petechiae; m) platelet count; n) bleeding time; o) conduction block; and p) inflammation (reduction of inflammation), compared to the level or degree of the outcome in the individual before treatment with the anti-C1s antibody.

In some cases, an anti-C1s antibody, when administered in a dose of 7.5 g, and when administered in one or more doses as monotherapy or in combination therapy to an individual having a complement-mediated disease or disorder, is effective to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation (reduction of thrombus formation); f) clotting (reduction of clotting); g) kidney function; h) hematocrit (red blood cell count); i) pruritis; j) blister formation; k) skin rash; l) petechiae; m) platelet count; n) bleeding time; o) conduction block; and p) inflammation (reduction of inflammation), compared to the level or degree of the outcome in the individual before treatment with the anti-C1s antibody.

In some cases, an anti-C1s antibody, when administered in a dose of 5.5 g, and when administered in one or more doses as monotherapy or in combination therapy to an individual having a complement-mediated disease or disorder, is effective to reduce complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the anti-C1s antibody.

In some cases, an anti-C1s antibody, when administered in a dose of 6.5 g, and when administered in one or more doses as monotherapy or in combination therapy to an individual having a complement-mediated disease or disorder, is effective to reduce complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the anti-C1s antibody.

In some cases, an anti-C1s antibody, when administered in a dose of 7.5 g, and when administered in one or more doses as monotherapy or in combination therapy to an individual having a complement-mediated disease or disorder, is effective to reduce complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the anti-C1s antibody.

In some cases, an anti-C1s antibody, when administered in a dose of 5.5 g, and when administered in one or more doses as monotherapy or in combination therapy to an individual having a complement-mediated disease or disorder, is effective to inhibit cleavage of complement component C4 in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of C4 cleavage in the individual before treatment with the anti-C1s antibody.

In some cases, an anti-C1s antibody, when administered in a dose of 6.5 g, and when administered in one or more doses as monotherapy or in combination therapy to an individual having a complement-mediated disease or disorder, is effective to inhibit cleavage of complement component C4 in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of C4 cleavage in the individual before treatment with the anti-C1s antibody.

In some cases, an anti-C1s antibody, when administered in a dose of 7.5 g, and when administered in one or more doses as monotherapy or in combination therapy to an individual having a complement-mediated disease or disorder, is effective to inhibit cleavage of complement component C4 in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of C4 cleavage in the individual before treatment with the anti-C1s antibody.

In some cases, an anti-C1s antibody, when administered at an effective dose, and when administered in one or more doses as monotherapy or in combination therapy to an individual having a complement-mediated disease or disorder, is effective to achieve and maintain a serum concentration of anti-C1s antibody of at least about 20 μg/mL, at least about 25 μg/mL, at least about 30 μg/mL, at least about 35 μg/mL, at least about 40 μg/mL, at least about 45 μg/mL, at least about 50 μg/mL, at least about 55 μg/mL, at least about 60 μg/mL, at least about 65 μg/mL, at least about 70 μg/mL, at least about 75 μg/mL, at least about 80 μg/mL, at least about 85 μg/mL, at least about 90 μg/mL, at least about 95 μg/mL, or at least about 100 μg/mL.

In some cases, an anti-C1s antibody, when administered at an effective dose, and when administered in one or more doses as monotherapy or in combination therapy to an individual having a complement-mediated disease or disorder, achieves and maintains a serum concentration of anti-C1s antibody to inhibit complement classical pathway (CP) by at least 50%, at least 60%, at least 70%, at least 80%, or at least 90%. In some case, an anti-C1s antibody, when administered in a dose of 5.5 g, and when administered in one or more doses as monotherapy or in combination therapy to an individual having a complement-mediated disease or disorder, is effective to inhibit CP by 90%. In some case, an anti-C1s antibody, when administered in a dose of 6.5 g, and when administered in one or more doses as monotherapy or in combination therapy to an individual having a complement-mediated disease or disorder, is effective to inhibit CP by 90%. In some case, an anti-C1s antibody, when administered in a dose of 7.5 g, and when administered in one or more doses as monotherapy or in combination therapy to an individual having a complement-mediated disease or disorder, is effective to inhibit CP by 90%.

In some cases, an anti-C1s antibody, when administered at an effective dose, and when administered in one or more doses as monotherapy or in combination therapy to an individual having a complement-mediated disease or disorder, achieves and maintains a serum concentration of anti-C1s antibody to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; (j) oligodendrocyte loss; (k) dendritic cell infiltration; (1) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; (x) blister formation; (y) pruritus; (z) skin rash; (ab) skin erosions; (ac) petechiae; (ad) bleeding time; (ae) conduction block; compared to the level or degree of the outcome in the individual before treatment with the anti-C1s antibody.

In some cases, an anti-C1s antibody, when administered at an effective dose, and when administered in one or more doses as monotherapy or in combination therapy to an individual having a complement-mediated disease or disorder, achieves and maintains a serum concentration of anti-C1s antibody to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation (reduction of thrombus formation); f) clotting (reduction of clotting); g) kidney function; h) hematocrit (red blood cell count); i) pruritis; j) blister formation; k) skin rash; l) petechiae; m) platelet count; n) bleeding time; o) conduction block; and p) inflammation (reduction of inflammation), compared to the level or degree of the outcome in the individual before treatment with the anti-C1s antibody.

In some cases, an anti-C1s antibody, when administered at an effective dose, and when administered in one or more doses as monotherapy or in combination therapy to an individual having a complement-mediated disease or disorder, achieves and maintains a serum concentration of anti-C1s antibody to reduce complement activation in the individual by at least about 10% at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the anti-C1s antibody.

In some cases, an anti-C1s antibody, when administered at an effective dose, and when administered in one or more doses as monotherapy or in combination therapy to an individual having a complement-mediated disease or disorder, achieves and maintains a serum concentration of anti-C1s antibody to inhibit cleavage of complement component C4 in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of C4 cleavage in the individual before treatment with the anti-C1s antibody.

In some cases, the effective dose of the anti-C1s antibody is at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

In some cases, the effective dose of the anti-C1s antibody is between about 60 mg/kg and about 100 mg/kg, about 60 mg/kg and about 95 mg/kg, about 60 mg/kg and about 90 mg/kg, about 60 mg/kg and about 85 mg/kg, about 60 mg/kg and about 80 mg/kg, about 60 mg/kg and about 75 mg/kg, about 60 mg/kg and about 70 mg/kg, or about 60 mg/kg and about 65 mg/kg. In some aspects, the effective dose of the anti-C1s antibody is between about 45 mg/kg and about 85 mg/kg, about 45 mg/kg and about 80 mg/kg, about 45 mg/kg and about 75 mg/kg, about 45 mg/kg and about 70 mg/kg, about 45 mg/kg and about 65 mg/kg, about 45 mg/kg and about 60 mg/kg, or about 45 mg/kg and about 50 mg/kg. In some cases, the effective dose of the anti-C1s antibody is between about 85 mg/kg and about 150 mg/kg, about 85 mg/kg and about 145 mg/kg, about 85 mg/kg and about 140 mg/kg, about 85 mg/kg and about 135 mg/kg, about 85 mg/kg and about 130 mg/kg, about 85 mg/kg and about 125 mg/kg, about 85 mg/kg and about 125 mg/kg, about 85 mg/kg and about 120 mg/kg, about 85 mg/kg and about 115 mg/kg, about 85 mg/kg and about 110 mg/kg, about 85 mg/kg and about 105 mg/kg, about 85 mg/kg and about 100 mg/kg, about 85 mg/kg and about 95 mg/kg, or about 85 mg/kg and about 90 mg/kg.

In some cases, the effective dose is about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, or about 150 mg/kg.

In some cases, the anti-C1s antibody is administered in an effective amount of at least 4 g, at least 4.5 g, at least 5 g, at least 5.5 g, at least 6 g, at least 6.5 g, at least 7 g, at least 7.5 g, at least 8 g, at least 8.5 g, at least 9 g, at least 9.5 g, or at least 10 g.

In some cases, the anti-C1s antibody is administered in an effective amount between about 5.5 g and about 10 g, about 5.5 g and about 9.5 g, about 5.5 g and about 9 g, about 5.5 g and about 8.5 g, about 5.5 g and about 8 g, about 5.5 g and about 7.5 g, about 5.5 g and about 7 g, about 5.5 g and about 6.5 g, or about 5.5 g and about 6 g. In some aspects, the anti-C1s antibody is administered in an amount between about 4.5 g and about 8.5 g, about 4.5 g and about 8 g, about 4.5 g and about 7.5 g, about 4.5 g and about 7 g, about 4.5 g and about 6.5 g, about 4.5 g and about 6 g, about 4.5 g and about 5.5 g, or about 4.5 g and about 5 g. in some aspects, the anti-C1s antibody is administered in an amount between about 7.5 g and about 12 g, about 7.5 g and about 11.5 g, about 7.5 g and about 11 g, about 7.5 g and about 10.5 g, about 7.5 g and about 10 g, about 7.5 g and about 9.5 g, about 7.5 g and about 9 g, about 7.5 g and about 8.5 g, or about 7.5 g and about 8 g.

Having now described the present disclosure in detail, the same will be more clearly understood by reference to the following examples, which are included herewith for purposes of illustration only and are not intended to be limiting of the disclosure.

### EXAMPLES

### Example 1

### An Anti-C1s Antibody that Rapidly Halts Hemolysis and Corrects Severe Anemia in Transfusion Dependent Primary Cold Agglutinin Disease Patients

This example provides a humanized anti-C1s antibody, BIVV009 (also known as TNT009), that provides clinical benefit for patients with cold agglutinin disease. This example provides clinical evidence that BIVV009 can rapidly stop hemolysis and restore normal hemoglobin levels in cold agglutinin disease patients.

### Methods:

Study Design: The trial protocol and its amendments were approved by the National Competent Authority and the Ethics Committee of the Medical University of Vienna, and the trial is registered at ClinicalTrials.gov (NCT 02502903) and EUDRACT (EUDRA-CT 2014-003881-26). This is a first-in-human trial using an integrated protocol design which studied single and multiple ascending doses (MAD) of BIVV009 in a randomized placebo controlled setting in healthy volunteers (Phase 1a), as well as a prospective open label trial design in four different diseases which share a common underlying pathophysiology, i.e.—antibody mediated complement activation (Phase 1b). This example focuses on the observed efficacy data of BIVV009 in the patient group suffering from cold agglutinin disease (treated from January until December 2016) and is augmented by the confirmation of these findings upon re-exposure to the drug under a named patient program.

Patients: Inclusion criteria comprised age ≥18 years old, previously vaccinated against encapsulated bacterial pathogens (Neisseria meningitidis, Haemophilus influenzae, and Streptococcus pneumoniae) or willing to undergo vaccination; able to comprehend and to give informed consent; able to co-operate, and a confirmed diagnosis of cold agglutinin disease (cold agglutinin titer >1:32) within the 3 months preceding enrolment. Exclusion criteria were active infection or history of the same within the preceding month; an autoimmune disorder other than cold agglutinin disease; other known complement-mediated disorders; known malignancy (other than locally limited, previously surgically removed basal cell carcinoma of the skin, lymphoproliferative disorders causally un-related to the complement-mediated diseases under study, etc.); clinically significant hepatobiliary disorder; history of infusion hypersensitivity; allergic or anaphylactic reactions to other therapeutic proteins; substance abuse; mental illness; women of child-bearing age not practicing contraception; concurrent treatment with other experimental drugs or participation in another clinical trial with any investigational drug within 30 days prior to treatment start, and a body weight >98 kg.

Treatment: BIVV009 is a humanized anti-C1s IgG₄ monoclonal antibody that has attained Orphan Drug Designation in the European Union and the US. Patients underwent a screening examination and could start drug infusions a minimum of 14 days after vaccinations against Neisseria meningitidis, Haemophilus influenzae, and Streptococcus pneumoniae. Upon request by the Austrian Agency for Health and Food Safety (AGES), an initial 10 mg/kg intravenous (IV) “test” dose of BIVV009 was infused in case of unforeseen adverse effects upon first infusion. One to 4 days later, patients received a full 60 mg/kg dose, followed by three additional 60 mg/kg infusions at weekly intervals. Patients were under constant observation by a physician and monitored with pulse-oximetry and regular blood pressure readings during the 1 hour infusions. Patients were followed for a total of 49-53 days under the protocol.

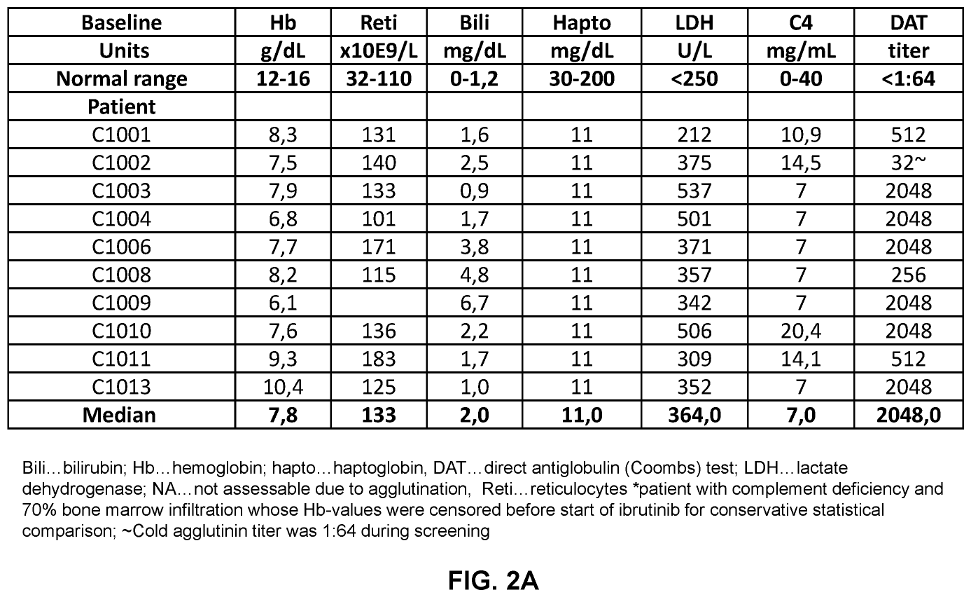

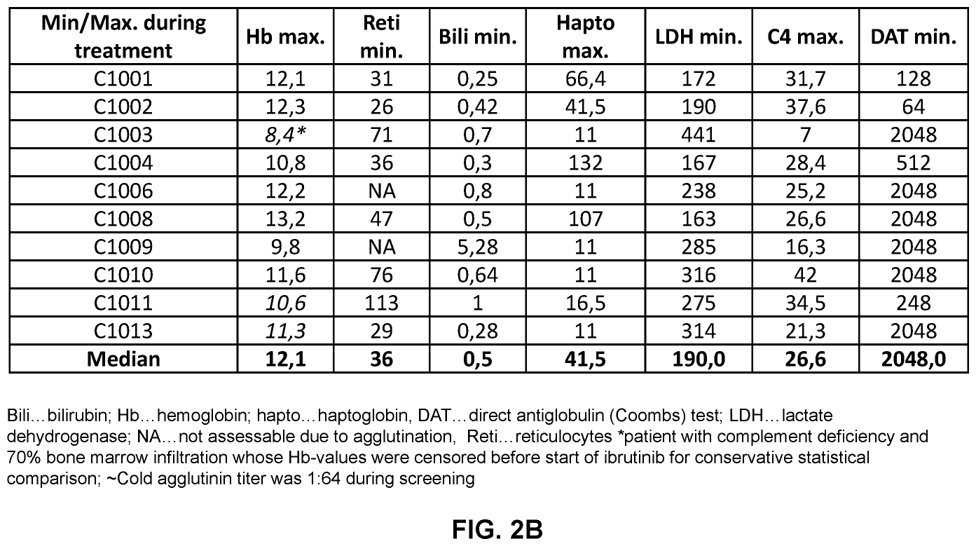

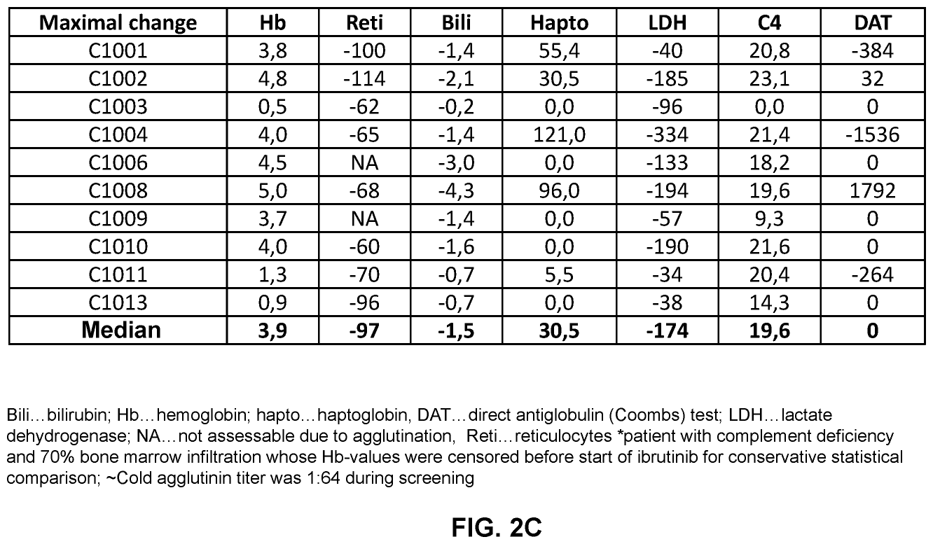

Laboratory analysis: All laboratory parameters were measured in a fully automated manner at the central laboratory of the Medical University of Vienna. Technicians additionally performed microscopic differential blood counts. All samples were collected by fresh venipunctures into 37° C. pre-warmed evacuated blood tubes and transported in a pre-warmed steel block to the central laboratory. In some instances, strong agglutination and ex vivo hemolysis of blood occurred after blood withdrawal which prevented accurate measurements of secondary outcome parameters. The direct antiglobulin test (DAT) was analyzed with LISS/Coombs Gelcards (Bio-Rad GmbH, Vienna, Austria). Serum BIVV009 levels were determined using a direct binding ELISA in which Cis coated plates were used to capture free BIVV009, detected using a goat anti-human HRP conjugated secondary antibody and developed with the colorimetric substrate 3,3′,5,5′-Tetramethylbenzidine (TMB). The Complement System Classical Pathway WIESLAB® (WL CP), an ELISA that detects ex vivo classical pathway mediated deposition of the membrane attack complex, was used to assess BIVV009 pharmacodynamic activity in serum samples (Euro-Diagnostica, Malmo, Sweden). Flow cytometry to detect C3d on patient erythrocytes was performed as described by Shi et al., Blood, 123(26):4015-22 (2014). A summary of laboratory parameters measured before treatment and maximal changes during treatment is shown in FIG. 2A-2C.

Statistical analysis: A sample size calculation was not performed for this pilot trial in CAD patients because no previous data were available to estimate effect size and variability thereof. The primary outcome variable of interest in CAD patients is the hemoglobin level because it determines symptoms, circulatory instability and is the main trigger for transfusions. Hemoglobin changes are expressed as 95% confidence intervals. Strong ex vivo red blood cell agglutination occasionally resulted in unmeasurable values for reticulocyte counts, lactate dehydrogenase and the DAT despite using sampling tubes preheated to 37° C. However, no missing data were imputed. Clinical response was defined as an increase in hemoglobin by ≥2 g/dL. No inferential statistical testing was planned, but a Friedman ANOVA was used for time courses, and a Wilcoxon test for differences between baseline and maximum individual effects. Other markers including DAT, bilirubin, reticulocyte counts, haptoglobin, lactate dehydrogenase, total complement activity (CH50) and C4 are non-independent secondary outcome parameters. Data are summarized descriptively using median and the range. In cases where values were below the detection limit of the assays, values of the detection limit minus 1 were assigned in graphs (e.g. haptoglobin=11 instead of <12 mg/dL; similarly, 1:2048 was assigned to cold agglutinin titers >1:1024). To increase the level of confidence for cause-effect relationship and to exclude regression to the mean, we studied the reversal of effects (i.e. recurrence of anemia and hemolysis) when drug washed out, and its recapitulation upon re-challenge. This concept was adapted from the guideline of Clinical Trials in Small Populations (CHMP/EWP/83561/2005) and represents a series of non-randomized n-of-1 trials with several cross-over periods from on-treatment to off-treatment. The duration of the off-treatment periods was mainly driven by the recurrence of hemolysis and severe anemia after discontinuation of treatment, with simultaneous avoidance of unnecessary transfusions between periods.

### Results:

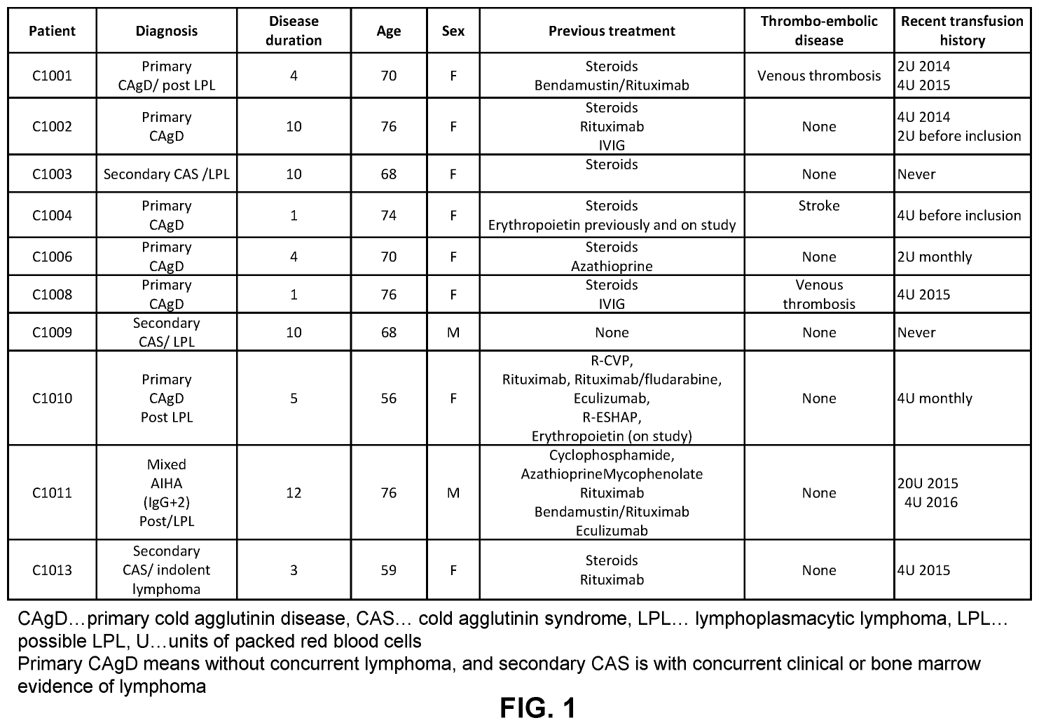

Study Population: Patient characteristics are shown in FIG. 1. Thirteen patients were screened; three females were excluded because of iron anemia and inactive cold agglutinin disease, negative cold agglutinin titer, or Hb levels >11 g/dL. Ten patients including 3 from the United Kingdom and 1 from Canada/Spain (8 Caucasians, 1 Hispanic, 1 Indian) were eventually included with a median disease duration of 5 years (range: 1-12 years). Three patients were referred to the trial while being on moderate doses of steroids (10-25 mg/day), which were reduced to <10 mg prednisolone on the first trial day and could be tapered and discontinued within the first weeks on BIVV009.

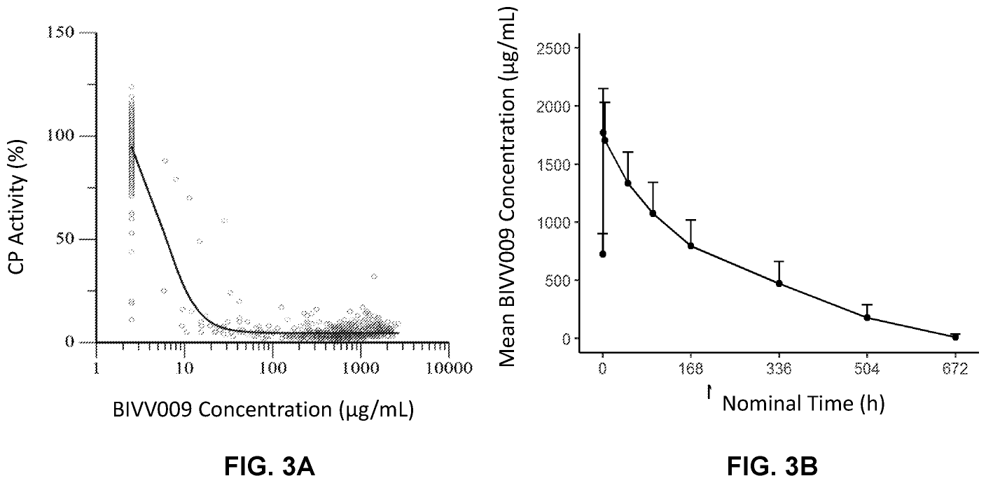

Pharmacokinetics and pharmacodynamics: The single ascending dose portion of the study performed in healthy volunteers demonstrated that BIVV009 undergoes non-linear elimination at concentrations lower than approximately 100 μg/mL; this behavior is frequently observed for other monoclonal antibodies, and suggests that target mediated elimination processes are involved. Using an ex vivo readout of serum classical pathway activity (WL CP ELISA, see Methods) we were able to assess the relationship between serum BIVV009 concentrations and serum classical pathway activity. Based on modeling in NHV subjects, a steep concentration-effect relationship was observed for the knockdown of classical pathway activity, reaching maximum effect (>90% inhibition of classical pathway activity) at BIVV009 concentrations of approximately 20 μg/mL (FIG. 3A). BIVV009 pharmacokinetics are similar regardless of disease status, as demonstrated by mean Cₘₐₓ and AUC results following four weekly 60 mg/kg doses in NHV (2073 μg/mL and 234612 μg*h/mL, respectively) and CAD patients (1885 μg/mL and 209996 μg*h/mL, respectively). As can be seen in the mean BIVV009 concentration-time profile in CAD patients (FIG. 3B), BIVV009 concentrations remain well above the 20 μg/mL level in patients who received four weekly 60 mg/kg doses, and only begins to approach this pharmacodynamic threshold 672h (28 days) after the last dose, implying that this dosing regimen is adequate for maintaining long-acting complement inhibition above the clinical effect threshold.

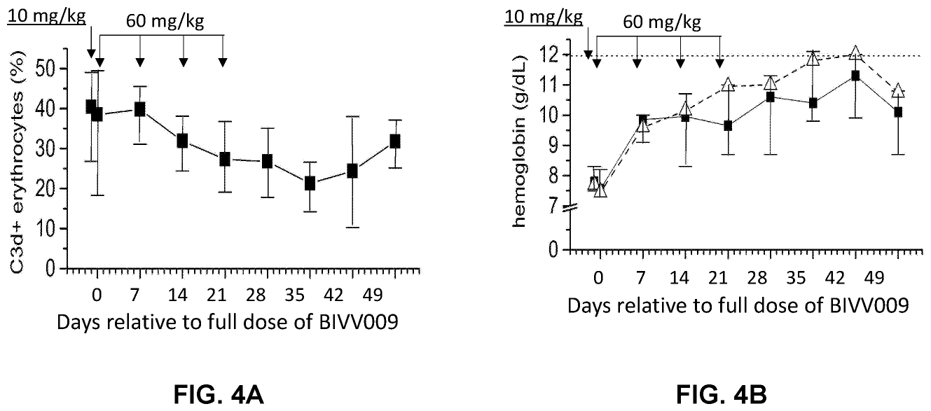

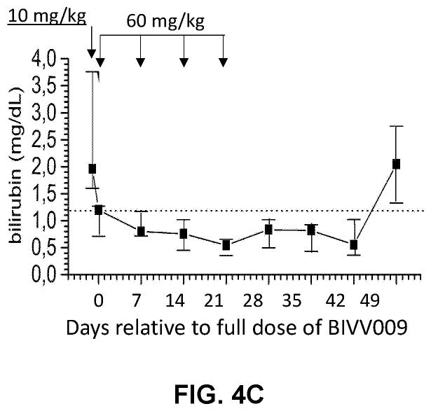

Consistent with the results observed using the WL CP ELISA, CH50, a measure of serum classical pathway activity, significantly reduced from pre-treatment levels within 24 h of BIVV009 administration (p=0.0209). Plasma levels of complement component C4, the first substrate cleaved by C1s, increased gradually over the course of the study, resulting in a median 3.8-fold increase (p=0.0077). Flow cytometry analyses revealed that the number of C3d positive erythrocytes significantly decreased from 40% (IQR: 27-49%) to a nadir of 21% (IQR: 14-27%) 5 weeks after the first dose (p<0.0172; FIG. 4A). These measures of in vivo BIVV009 pharmacodynamic activity on C4 levels, and more importantly, on the red blood cell surface, are consistent with its mechanism of action and suggest that BIVV009 inhibits the classical complement pathway in CAD patients.

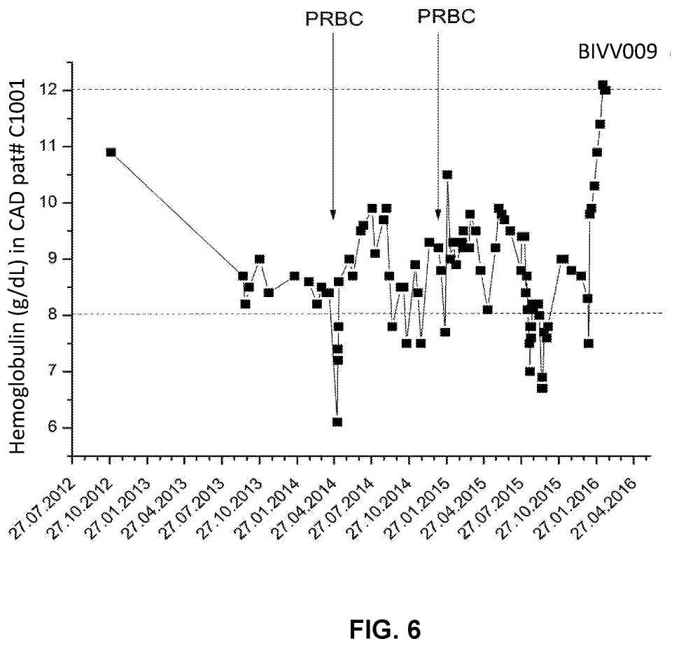

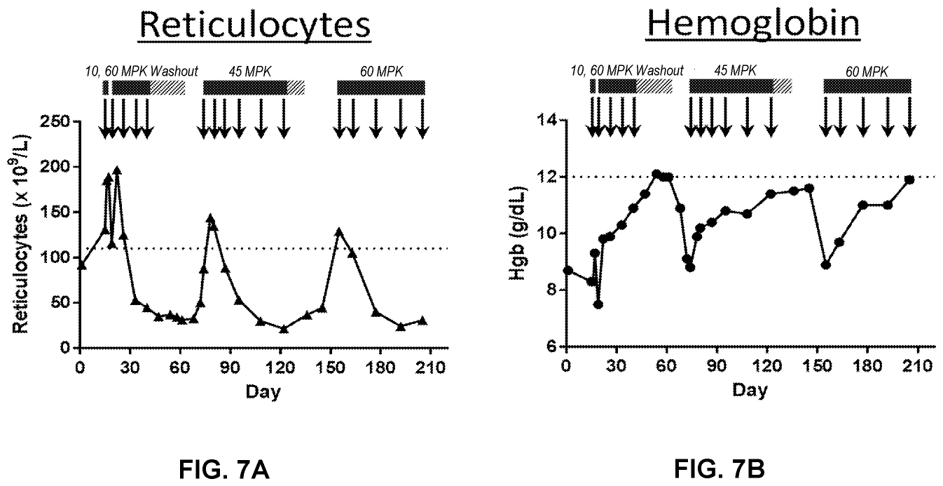

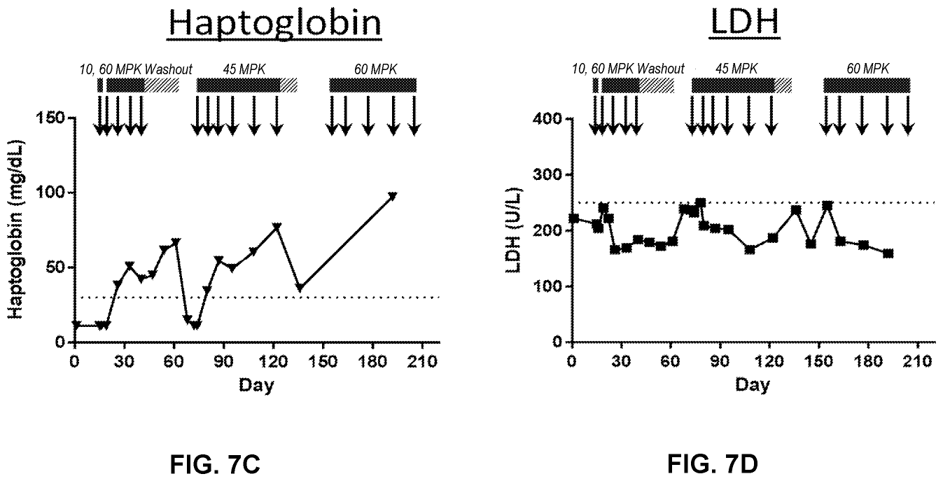

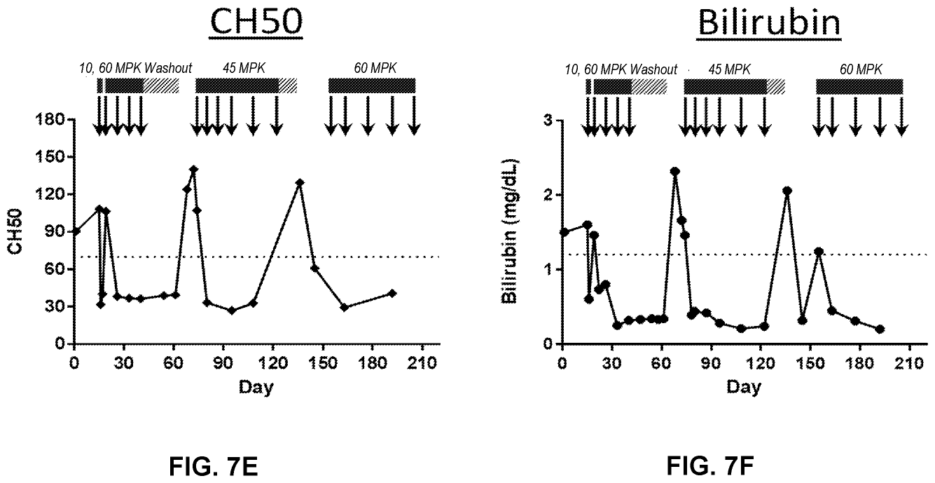

The present example further shows that BIVV009 increases hemoglobin levels and rapidly inhibits hemolysis in CAD patients. BIVV009 infusion resulted in a median hemoglobin increase of 1.6 g/dL within the first week of treatment (p=0.0069; n=10), and by a median best response of 3.9 g/dL (IQR: 1.3-4.5; 95% CI: 2.1-4.5; p=0.0050; n=10) after 6 weeks (FIG. 4B). Furthermore, hemoglobin completely normalized in four patients during the limited trial duration (≥12 g/dL), and 5 patients experienced a >4 g/dL increase. Historical hemoglobin data (pre-BIVV009 administration) for one patient is shown in FIG. 6 to demonstrate the clinical efficacy of BIVV009 in a chronically anemic CAD patient. PRBC denotes when transfusion support was provided. These historical hemoglobin data, which include over four years of hemoglobin values prior to enrollment in the trial, demonstrate that hemoglobin levels in this patient never approached the lower limit of normal (12 g/dL, dotted line) until BIVV009 was provided. Hematological data in FIG. 7 demonstrate the clinical benefit provided by BIVV009 to the same patient in a series of on- (denoted by solid horizontal bars) and off-treatment (denoted by no bars) periods (hashed bars represent BIVV009 washout period). FIG. 7A shows that BIVV009 administration results in an immediate increase of reticulocytes, suggesting that BIVV009 prevents reticulocyte destruction. FIG. 7B shows that BIVV009 increased hemoglobin levels by 3.8 g/dL. FIG. 7C shows that BIVV009 increases haptoglobin levels to within the normal range, compared to before treatment when haptoglobin was below the limit of detection. FIG. 7D shows that LDH levels, a marker of intravascular hemolysis, decreased upon BIVV009 treatment. FIG. 7E shows that BIVV009 reduced CH50 levels, a measure of serum classical pathway activity. Finally, FIG. 7F shows that BIVV009 reduced bilirubin levels, suggesting that the drug stops extravascular hemolysis. The modulation for all these markers reversed following BIVV009 washout (periods denoted by no bars) and recurred upon retreatment (solid horizontal bars). Together, these data demonstrate that BIVV009 prevents cold agglutinin mediated complement destruction of erythrocytes and reticulocytes in a CAD patient.

Reticulocyte counts increased by a median of 41% within the first 24 hours (p=0.0381, n=10), which then gradually declined to within the normal range as expected upon rising levels of hemoglobin. All five patients who had been transfusion-dependent prior to enrolment were transfusion free throughout their treatment course with BIVV009. FIG. 6 shows historical hemoglobin levels for a patient with CAD who received packed red blood cells (PRBC) transfusions prior to beginning treatment with BIVV009. FIG. 7A-7F show biochemical response patterns for reticulocytes, hemoglobin, haptoglobin, lactate dehydrogenase (LDH), total complement activity (CH50), and bilirubin levels in a patient with CAD upon repeat administration of BIVV009.

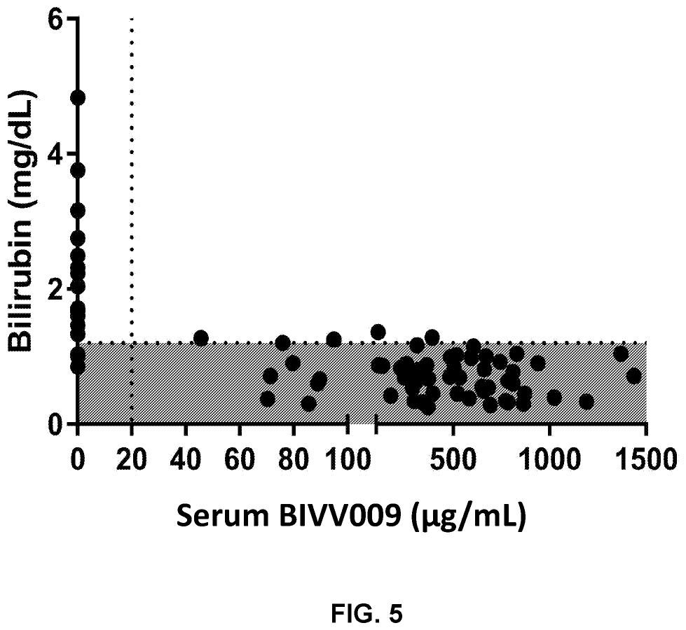

Haptoglobin, below the level of detection (<11 mg/dL) in all patients before treatment, normalized in four patients within 1-2 weeks and confirmed the complete inhibition of hemolysis. Pre-dose bilirubin levels were found to be elevated in 7 CAD patients, suggestive of an increased erythrocyte turnover by the mononuclear phagocyte system. BIVV009 administration resulted in a median decrease of bilirubin levels by 61% within 24 hours of the first infusion (p=0.0068, n=10; FIG. 4C), normalizing in 6 patients. Similarly, upon BIVV009 washout, bilirubin levels increased significantly, demonstrating the recurrence of hemolysis. The rapidity of the reduction and normalization of circulating bilirubin upon BIVV009 treatment in addition to its reappearance following washout provided a disease-related biomarker to examine the relationship between BIVV009 and extravascular hemolysis. At BIVV009 concentrations greater than 20 μg/mL, the threshold above which the drug completely inhibits serum classical pathway activity (FIG. 3A), bilirubin levels were within the normal range, with few exceptions. Conversely, at concentrations below 20 μg/mL bilirubin levels, bilirubin tended to be above the normal range (FIG. 5).

Response analysis: Seven of 10 CAD patients derived clinical benefit defined as a hemoglobin increase >2 g/dL, including patients failing to respond or relapsing after rituximab (C1002), rituximab plus bendamustin (C1001; C1010), or eculizumab (C1010). Three patients did not respond sufficiently to treatment with BIVV009 (0.5-1.3 g/dL increase in hemoglobin). One patient (C1011) was repeatedly positive for both C3d and IgG (>1+) in the Coombs test, suggesting that he suffered from both cold and warm autoimmune hemolytic anemia (i.e.—mixed autoimmune hemolytic anemia). The two other patients had active lymphoma with lymphocytic bone marrow infiltrates of 70% and 15% (C1003 and C1013, respectively), and one further patient with 60% bone marrow infiltration only partially responded (C1009). Lactate dehydrogenase (LDH) levels also normalized in 5 of the responders, whereas LDH increased 2-3-fold in 2 of the 3 non-responders.

Recapitulation of response following washout and re-challenge of BIVV009: Complement deposition on erythrocytes, anemia, and hemolysis recurred in all responders when BIVV009 levels dropped below the pharmacodynamic threshold of 20 μg/mL approximately 3-4 weeks after the last dose of BIVV009 (FIG. 3B, FIG. 4). Therefore, responders were offered the opportunity to participate in a named patient program to prove causality by serial treatments in a series of n-of-1 trials in six patients. The patient with the partial response preferred to continue without treatment (as the patient was travelling back and forth from the UK) and his hemoglobin level decreased from 8.7 g/dL while on BIVV009 to 6-6.5 g/dL during follow up. In the remaining six patients, re-exposure to BIVV009 recapitulated the immediate onset of effect, and the rapid and complete inhibition of hemolysis.

As pharmacokinetic analyses demonstrated that weekly 60 mg/kg doses resulted in increasing trough levels of BIVV009, suggesting accumulation of the drug (FIG. 2), alternative doses and dose regimens were explored in the named patient program. Two patients received 4 weekly doses of 45 mg/kg BIVV009 followed by 45 mg/kg every other week. This was abandoned after 7 infusions because laboratory parameters showed breakthrough hemolysis. Breakthrough was accompanied by restoration of serum CH50 activity and undetectable circulating BIVV009, confirming that breakthrough was a result of inadequate trough concentrations. Patients were then further supported by two weekly loading doses of 60 mg/kg BIVV009 followed by 60 mg/kg every other week which again led to breakthrough hemolysis in 2 patients after 8 infusions. An increase of the dose to 65 mg/kg, or a fixed dose of 5.5 g every other week prevented further breakthrough events. All 5 transfusion dependent patients achieved normal hemoglobin levels (>12 g/dL) following BIVV009 treatment at least once, and remained transfusion free while on treatment. Two patients discontinued the named patient program for reasons other than drug safety or efficacy and have again become transfusion-dependent, requiring transfusion support approximately every other week.

Safety: All infusions were well tolerated without premedication and without relevant drug related adverse effects. There were few adverse events during the trial; all were mild or moderate and considered unrelated or unlikely related to study drug. Patient C1001 had nausea and vomiting on two occasions, once associated with diarrhea. Patient C1002 complained of night sweats and developed a new vertebral fracture in addition to pre-existing ones attributed to long term steroid therapy. This eventually led to a planned hospitalization for the treatment of bone pain several weeks after the end of her participation in the trial. Patient C1004 complained of pruritus and had an exanthema, which was transient despite continuing BIVV009 exposure.

### Conclusion:

These data show that Cis blockade by the anti-C1s monoclonal antibody BIVV009 rapidly corrects severe, transfusion-dependent anemia in patients suffering from primary cold agglutinin disease.

### Example 2

### An Anti-C1s Monoclonal Antibody in Late Antibody-Mediated Kidney Allograft Rejection—Results from a First-in-Human Phase 1 Trial

This example provides a humanized anti-C1s antibody, BIVV009 (also known as TNT009), that provides clinical benefit for kidney transplant patients with antibody mediated rejection (ABMR). This example provides clinical evidence that BIVV009 effectively blocks alloantibody-triggered classical complement pathway (CP) activation in kidney allografts.

This single-arm phase 1b trial was designed to investigate the safety/tolerability profile and the complement inhibitory activity of a limited course of BIVV009 in kidney transplant recipients on maintenance immunosuppression. Here we describe the CP-blocking potential of BIVV009 and the morphologic and molecular evaluation of systematic follow-up biopsies from patients with active ABMR and evidence of CP activation (in vivo C4d staining and/or detection of complement-fixing donor-specific antibody (DSA) in serum) treated with BIVV009.

### Materials and Methods:

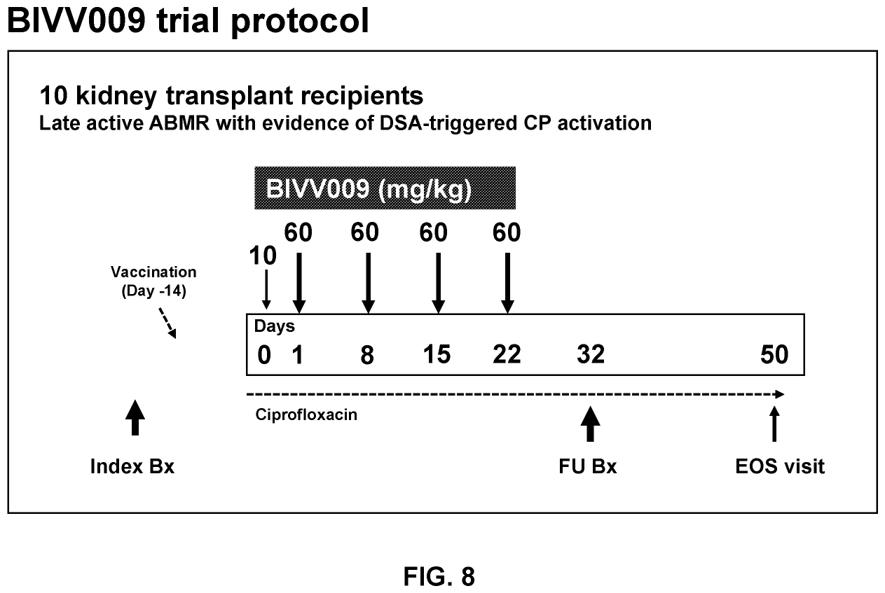

Study design and objectives. This prospective phase 1 trial included a single cohort of ten kidney transplant recipients diagnosed with late acute or chronic active ABMR. The study was part of a phase 1 basket trial designed to assess the safety, tolerability and potential efficacy of the humanized monoclonal anti-C1s antibody BIVV009 (formerly TNT009; Bioverativ Therapeutics, Inc., South San Francisco, Calif.) in healthy volunteers and patients with various diseases believed to be mediated by the CP (cold agglutinin disease, warm autoimmune hemolytic anemia, bullous pemphigoid and ABMR). The trial was registered at ClinicalTrials.gov (NCT 02502903) and EUDRACT (EUDRACT number: 2014-003881-26). We hypothesized that BIVV009 would be safe and well-tolerated, and able to effectively block CP activity in patients with ABMR. The trial was carried out at the Department of Clinical Pharmacology (Medical University of Vienna). No classical sample size estimation was performed. The study was approved by the ethics committee of the Medical University Vienna and was carried out in compliance with Good Clinical Practice Guidelines and in accordance with the principles of the Declaration of Helsinki and the Declaration of Istanbul. The design of the study is illustrated in FIG. 8.

Study patients: The trial included ten adult kidney transplant recipients diagnosed with late ABMR. Subjects were recruited between December 2015 and September 2016 at the nephrology outpatient clinic of the Medical University of Vienna, and the study was completed in November 2016. All participants provided written informed consent before enrollment. Key inclusion criteria were the ability to comprehend and to give informed consent, an age ≥18 years, a functioning allograft with an estimated glomerular filtration rate (eGFR) ≥20 mL/min/1.73m2 ≥180 days post-transplantation, detection of one or more anti-HLA class I and/or II DSA in serum, biopsy-proven, late ABMR (acute or chronic) showing morphological features of an active rejection process (g score >0, ptc score >0), a molecular biopsy signature of ABMR determined by the Molecular Microscope Diagnostic System 18 (MMDx; molecular ABMR score ≥0.20), and signs of CP activation (complement-fixing DSA and/or C4d deposition in index biopsies). Female subjects had to be post-menopausal, surgically sterilized or willing to use highly effective methods of birth control throughout the study and for 30 days after the end-of-study visit. Key exclusion criteria were acute allograft dysfunction and/or any rejection treatment within four weeks before study inclusion, the diagnosis of TCMR, or a contraindication to antibiotic prophylaxis with oral ciprofloxacin. Other exclusion criteria were as follows: an active acute or chronic viral, bacterial, fungal or mycobacterial infection or a history of same within the preceding month, an autoimmune disorder or known malignancy, a clinically significant hepatobiliary disorder, a history of infusion hypersensitivity, allergic or anaphylactic reactions to other therapeutic proteins, substance abuse, mental illness or other reasons that made it unlikely for the subject to comply fully with the study procedures, females who are pregnant, lactating, or potentially unreliable with respect to contraceptive practice, a body weight >98 kg, and participation in another clinical trial within 30 days prior to treatment start.

Trial medication: For drug administration patients were admitted to the study unit (Department of Clinical Pharmacology, Medical University Vienna). For safety reasons, patients received an initial 10 mg/kg test dose of BIVV009 one day before the first full dose. Treatment consisted of four weekly doses of 60 mg/kg. BIVV009 was administered via the intravenous route over 60 min. Before dosing, all subjects underwent vaccination against encapsulated bacteria (Neisseria meningitidis, Haemophilus influenzae, Streptococcus pneumoniae) followed by prophylaxis with ciprofloxacin (250 mg orally twice daily) throughout the whole study period.

Outcome measures: Study endpoints were evaluated until day 50 (end-of study visit). The primary endpoint was the safety and tolerability of BIVV009, evaluating the incidence and severity of adverse events (AE) defined and classified according to the International Conference on Harmonization (ICH) Guidelines for Good Clinical Practice. The protocol did not include an interim analysis. However, the trial was guided by an ongoing safety review process. Safety reviews were conducted in consultation with an independent data safety monitoring board, with the option of study interruption in case of unexpected clinical and laboratory findings raising safety concerns. Secondary endpoints included the pharmacokinetics of BIVV009, the ability of BIVV009 to block the CP in serum (overall CP activity and DSA-triggered CP activation) and in the transplanted kidney (capillary C4d deposition), the effect of BIVV009 on DSA mean fluorescence activity and C1q-fixing capability, and eGFR (Mayo equation) and spot urine protein/creatinine ratio. To assess the effect of BIVV009 on antibody-triggered inflammation/injury and gene expression patterns, patients underwent follow-up protocol biopsies at day 32.

Antibody and complement detection: Antibody and complement assays were performed in samples taken at twelve consecutive times: at day 0 (one hour before administration of the initial test dose of BIVV009), one hour before each full dose of BIVV009 at days 1, 8, 15 and 22, and at days 29, 36, 43 and 50 (end-of study visit). Sera were aliquoted and stored at −80° C. until analysis, without repeated freezing and thawing. To avoid inter-test variations in results, sera from all time points were assayed retrospectively, after the study was completed.

For Luminex-based detection of IgG type DSA, we used LABScreen HLA class I and II single-antigen flow bead (SAFB) assays (One Lambda, Inc, Canoga Park, Calif., USA). Sera were heat inactivated (56° C. for 30 minutes) to prevent complement-dependent interference. The threshold for DSA positivity was set at an MFI value ≥1,000. Alloreactivity patterns were analyzed using HLA Fusion 3.0 software (One Lambda) and donor specificity was evaluated in the context of the results of serological and/or low- or high-resolution donor/recipient HLA typing (HLA-A, -B, -Cw, -DR, -DQ and/or DP) retrieved from the database of the local HLA lab or Eurotransplant. For each sample, individual DSA and the DSA with the highest IgG MFI (MFI_max) were recorded.

The ability of detected DSA to bind recombinant C1q was assessed on SAFB using the C1qScreen assay following the manufacturer's instructions (One Lambda), setting the MFI threshold for C1q positivity to ≥500.

DSA-triggered activation of key component C3 was assessed by measuring the deposition of the C3 complement split product C3d to SAFB (patient serum as complement source) following an earlier described protocol. In brief, patient sera were incubated for 30 min with SAFB at room temperature and then incubated with a biotin-conjugated monoclonal antibody against human C3d (4 μg/mL; Quidel, San Diego, Calif., USA) for another 30 min. Subsequently, phycoerythrin-conjugated streptavidin (1 μg/mL; eBioscience, San Diego, Calif., USA) was added for 30 minutes. The threshold for C3d positivity was set to an MFI ≥100.

For assessment of overall CP complement activity, two different assays principles were applied. The Complement system Classical Pathway WIESLAB® assay to assess CP-triggered membrane attack complex activation was performed following manufacturer's instructions (Euro-Diagnostica, Malmö, Sweden). In parallel, the ability of sera from patients dosed with BIVV009 to deposit complement in an earlier described solid-phase assay that specifically detects third-party HLA antibody-triggered C3 activation was evaluated. In brief, patient sera were incubated with a mixture of HLA haplotype-coated LABSCREEN Mixed beads (One Lambda) spiked with high level complement-activating HLA antibodies (preincubation with a pool of heat-inactivated sera obtained from three broadly sensitized patients, of which each had a >99% virtual panel reactivity in SAFB assays). After washing, beads were stained with biotin-conjugated anti-C3d antibody and PE-conjugated streptavidin as described above. Assay results were recorded as normalized C3d MFI, whereby raw MFI obtained with a heat-inactivated non-binding negative control serum were subtracted from the MFI obtained with the patient serum MFI (normalized MFI). For each test serum, we calculated the mean value of normalized C3d MFI recorded on the 12 HLA class I and four of the five HLA class II bead populations within the LABSCREEN Mixed bead panel [one HLA class II bead population (ID25) was excluded because of consistently negative C3d staining, MFI<100].

Biopsies. Renal allograft biopsies were performed using a 16-gauge needle. For light microscopy, electron microscopy and gene expression analysis, two cores were obtained. For immunohistochemical C4d staining we applied a polyclonal anti-C4d antibody (BI-RC4D, Biomedica, Vienna, Austria). C4d was scored 0 (negative), 1 (minimal), 2 (focal), and 3 (diffuse), respectively. Minimal staining (C4d1) along peritubular capillaries (PTC) was considered as positive. For gene expression analysis, a 3-mm proportion of a biopsy core was placed in RNAlater, stored at −20° C. and shipped at room temperature to the Alberta Transplant Applied Genomics Centre (ATAGC, University of Alberta, Edmonton, AB, Canada). RNA extraction and gene expression analysis were performed using PrimeView GeneChip arrays (Affymetrix Santa Clara, Calif., USA), as previously described in detail. Classifiers related to rejection (ABMR, TCMR, all Rejection) or acute kidney injury (AKI score) were generated on the basis of a reference set of 1208 biopsy specimens 21. In addition, scores of various pathogenesis-based transcripts (PBT), which represent major biological events derived from experimental cell culture studies, mouse transplant studies and human kidney transplants, and were shown to be involved in diverse annotated pathologic processes (e.g. cytotoxic T cell infiltration, y-interferon effects, natural killer cell burden, epithelial damage) were evaluated as earlier described in detail. Following the 2013 update of the Banff classification (Haas et al., Am J Transplant, 14(2):272-283 (2014)), ABMR was defined on the basis of histomorphologic, immunohistologic (C4d), ultrastructural (multilayering of PTC basement membranes), serological (DSA detection) criteria and a thoroughly validated molecular classifier for ABMR (molecular ABMR score ≥0.2) 18, respectively.

Statistical analysis. Continuous data are given as the median, interquartile range (IQR) and range. Discrete data are presented as counts and percentages. For paired sample comparison, the Wilcoxon signed-rank test was used. A two-sided p-value <0.05 was considered statistically significant. Analyses were performed using GraphPad Prism 6.0 (GraphPad Software Inc., San Diego, Calif., USA) and IBM SPSS Statistics 24 (IBM Corporation, Armonk, N.Y., USA).

### Results:

This phase 1 pilot trial included ten kidney transplant recipients diagnosed with late anti-HLA DSA-positive active ABMR associated with signs of antibody-triggered CP activation (complement-fixing DSA and/or C4d staining in PTC). ABMR was diagnosed after median of 4.3 years post-transplantation, and the first study visit was carried outa median of 38 (IQR: 28-45) days after the index biopsy. As shown in FIG. 8, all included patients received BIVV009 at a 10 mg/kg test dose, followed by 4-weekly doses of 60 mg/kg, and were subjected to follow-up biopsies 32 days after the first infusion. Baseline characteristics and data are provided below in Table 2.

TABLE 2
Baseline demographics and patient characteristics

| Parameters | Study cohort (n = 10) | |
|---|---|---|
| Variables recorded at the time of Tx | | |
| Recipient age (years), median (IQR, range) | 44 | (38-63; 27-73) |
| Donor age (years), median (IQR, range) | 60 | (53-63; 27-69) |
| Male recipient sex (%) | 6 | (60) |
| Live donor, n (%) ^{a} | 2 | (20) |
| ABO-incompatible live donor transplant, n (%) | 1 | (10) |
| Prior kidney transplant, n (%) | 3 | (30) |
| Combined pancreas/kidney transplant, n (%) | 1 | (10) |
| HLA mismatch in A, B and DR, median (IQR, range) | 4 | (3-5; 3-5) |
| Current CDC panel reactivity ≥10%, n (%) | 2 | (20) |
| Pre-transplant DSA, n (%)^{b} | 3 | (50) |
| IL-2R antibody, n (%) | 4 | (40) |
| Induction with antithymocyte globulin, n (%) | 3 | (30) |
| Peri-transplant immunoadsorption, n (%) | 3 | (30) |
| Tacrolimus-based triple immunosuppression, n (%) | 9 | (90) |
| Cyclosporine A-based immunosuppression, n (%) | 1 | (10) |
| Variables at the time of Bx/Study inclusion | | |
| Time to index-biopsy (years), median (IQR, range) | 4.3 | (2.9-8.0; 1.2-11.5) |
| Recipient age (years), median (IQR, range) | 51 | (46-66; 36-77) |
| Maintenance immunosuppression, n (%) | | |
| Tacrolimus, MPA, steroids | 9 | (90) |
| Cyclosporine A, MPA, steroids | 1 | (10) |
| Serum creatinine (mg/dL), median (IQR, range) | 1.7 | (1.4-2.4; 1.2-2.8) |
| eGFR (mL/min/1.73 m²), median (IQR, range) | 46 | (27-61; 24-78) |
| Urinary protein/creatinine ratio (mg/g), median (IQR, range) | 399 | (162-861; 0-6649) |

Two of the study patients were recipients of a living donor transplant, of which one was ABO-incompatible. Three recipients had been subjected to a protocol of peni-transplant immunoadsorption because of preformed DSA. At study inclusion, nine patients were on tacrolimus mycophenolic acid and steroids. Median eGFR was 46 mL/min/1.73m2 and urinary protein/creatinine ratio was 399 mg/g. Table 3 provides immunological, morphological and molecular results obtained at baseline.

TABLE 3
Baseline DSA characteristics and biopsy results

| Parameter | Study patients (n = 10) | |
|---|---|---|
| DSA characteristics at day 0 | | |
| HLA class I DSA only, n (%) | 3 | (30) |
| HLA class II DSA only, n (%) | 4 | (40) |
| HLA class I and II DSA, n (%) | 3 | (30) |
| Anti-DQ DSA, n (%) | 5 | (50) |
| Number of DSA, median (IQR, range) | 1 | (1-2; 1-6) |
| Characteristics of the immunodominant DSA | | |
| IgG MFI, median (IQR) | 7,814 | (1,836-14,659) |
| C1q binding (C1q MFI >500), n (%) | 6 | (60) |
| C1q MFI of C1q-fixing DSA, median (IQR) | 17,472 | (8,136-19,577) |
| C3d binding (C3d MFI >100), n (%) | 7 | (70) |
| C3d MFI of C3d-fixing DSA, median (IQR) | 195 | (140-1,284) |
| Biopsy results at baseline (index biopsy) | | |
| Morphological ABMR lesions and scores | | |
| Glomerulitis (g score ≥1), n (%) | 10 | (100) |
| g score, median (IQR, range) | 2 | (2.0-2.75; 1-3) |
| Peritubular capillaritis (ptc score ≥1), n (%) | 10 | (100) |
| ptc score, median (IQR, range) | 2 | (1.25-2.0; 1-3) |
| g + ptc sum score, median (IQR, range) | 4 | (4.0-4.75; 2-5) |
| Transplant glomerulopathy (eg score ≥1), n (%) | 9 | (90) |
| cg score, median (IQR, range) | 2 | (1.25-2.75; 0-3) |
| C4d in PTC (C4d score ≥1), n (%) | 8 | (80) |
| C4d score, median (IQR, range) | 2 | (2-3; 0-3) |
| High-grade MLPTC, n (%) | 3 | (30) |
| Molecular classifiers | | |
| ABMR score, median (IQR, range) | 0.78 | (0.49-0.96; 0.23-0.99) |
| TCMR score, median (IQR, range) | 0.01 | (0.0-0.03; 0.0-0.29) |
| all Rejection score, median (IQR, range) | 0.75 | (0.62-0.85; 0.31-0.94) |
| Atrophy/Fibrosis score, median (IQR, range) | 0.41 | (0.31-0.69; 0.07-0.88) |
| AKI score, median (IQR, range) | 0.19 | (0.02-0.67; −0.17-0.87) |
| Banff 2013 rejection types and categories | | |
| Chronic/active ABMR, n (%) | 9 | (90) |
| Acute/active ABMR, n (%) | 1 | (10) |
| C4d-positive ABMR, n (%) | 8 | (80) |
| Banff borderline lesion, n (%) | 1 | (10) |

ABMR, antibody-mediated rejection; DSA, donor-specific antibody; IQR, interquartile range; MFI, mean fluorescence intensity; MLPTC, multilayering of peritubular capillary basement membranes.
^{a}Material for electron microscopy was available for nine patients.

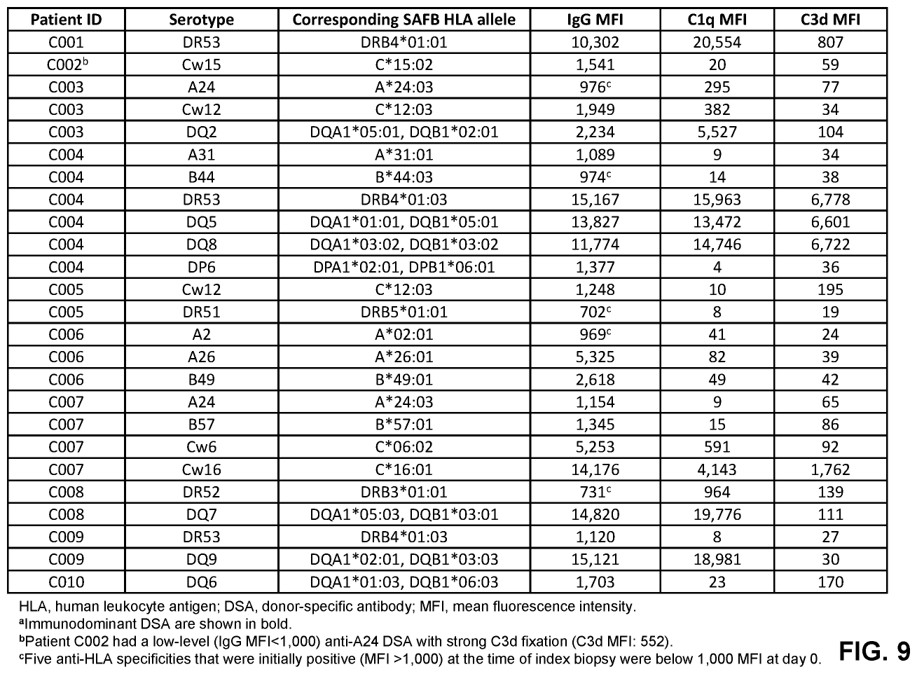

Immunodominant DSA were primarily directed against HLA class II antigens (seven patients; anti-HLA DQ reactivity: n=5). Six of the patients showed significant C1q-, and seven C3d-binding of the immunodominant DSA. Individual DSA specificities identified at the time of study inclusion are detailed in FIG. 9. Nine study patients showed chronic/active, one acute/active ABMR. Eight ABMR cases were C4d-positive. None of the patients had T cell-mediated rejection, and one index biopsy showed borderline changes. A sum score of glomerulitis and peritubular capillaritis (g+ptc score) was in median 4, and the transplant glomerulopathy (cg) score was 2. Median molecular ABMR and all Rejection scores were 0.78 and 0.75, respectively (Table 3).

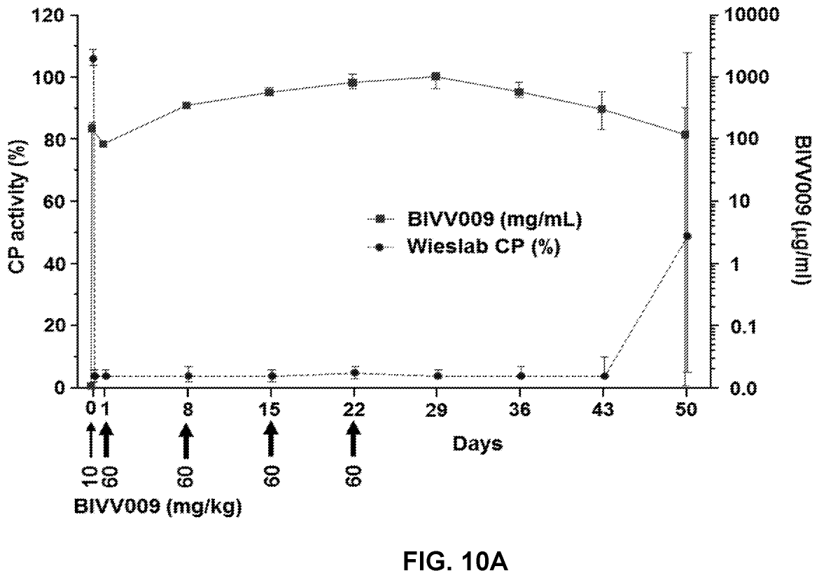

Impact of BIVV009 on CP activity in serum: BIVV009 treatment led to a complete and sustained blockade of serum CP activity detected in WIESLAB® assays (CP-triggered formation of the membrane attack complex) (FIG. 10A). Four weeks after the last infusion median CP activity was still below 50%. As shown in FIG. 10A, CP inhibition was thereby closely related to serum concentrations of BIVV009. A comparable effect was observed for a bead assay assessing HLA antibody-triggered C3 cleavage. As illustrated in FIG. 10B, BIVV009 did not affect the MFI or C1q-fixing capability of the immunodominant DSA. However, DSA-triggered C3 activation was virtually completely inhibited.

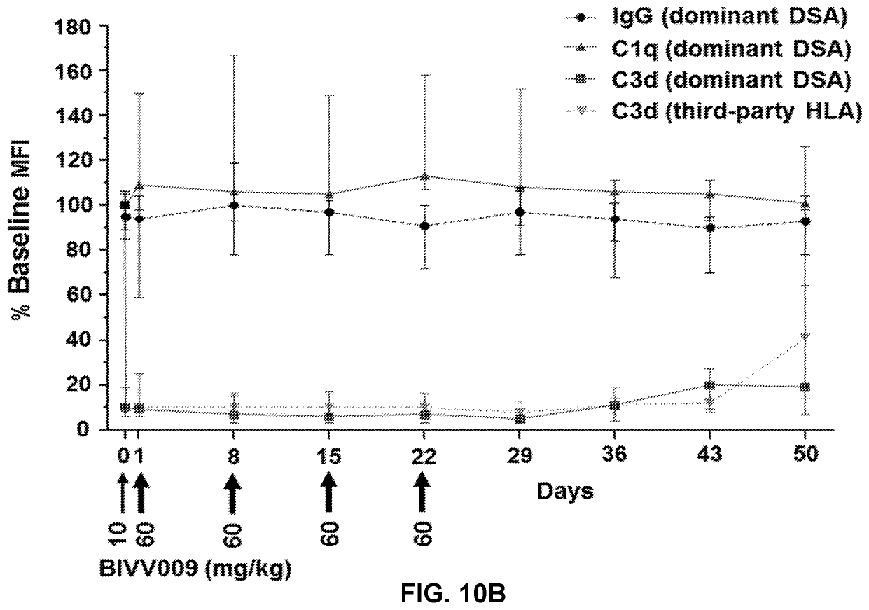

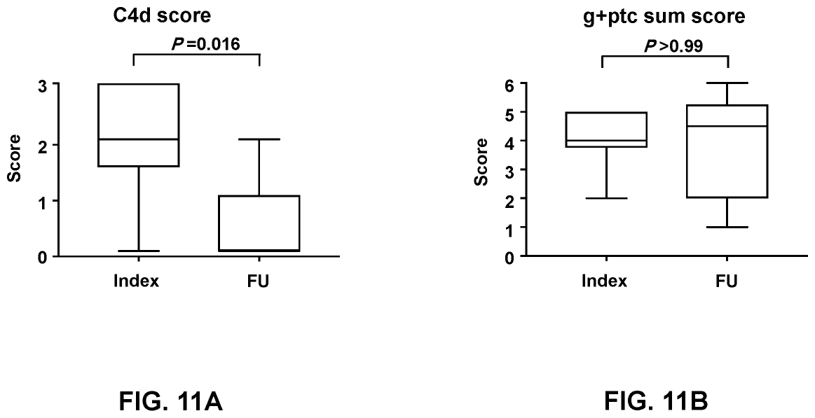

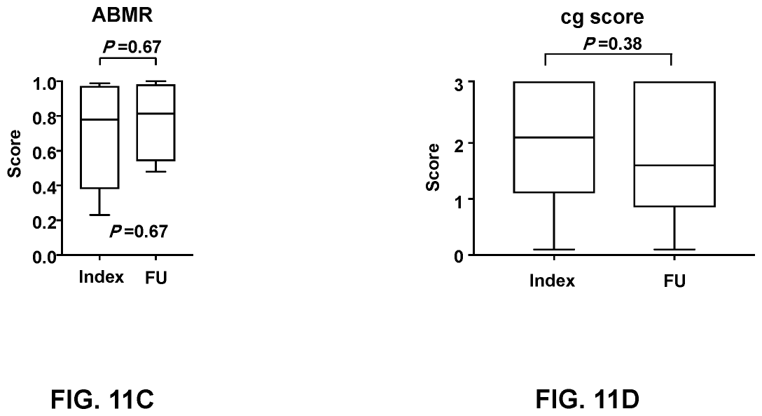

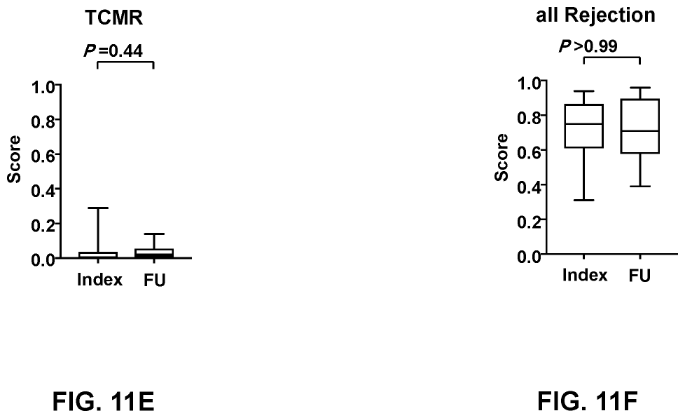

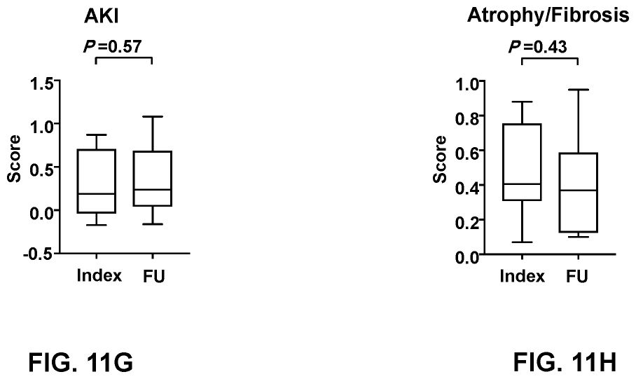

Impact of BIVV009 on C4d deposition in PTC: Follow-up biopsies performed 32 days after the first infusion indicated a significant decrease in median C4d scores: 2 (IQR: 2-3) in index vs. 0 (0-1) in follow-up biopsies (p=0.016) (FIG. 11A). Five recipients, of which three showed a diffuse staining pattern (C4d3), turned completely C4d-negative, and two showed only minimal staining (C4d1) in their follow-up biopsies. In a single case of an ABO-incompatible allograft focal staining (C4d2), however, no change in C4d staining was observed.

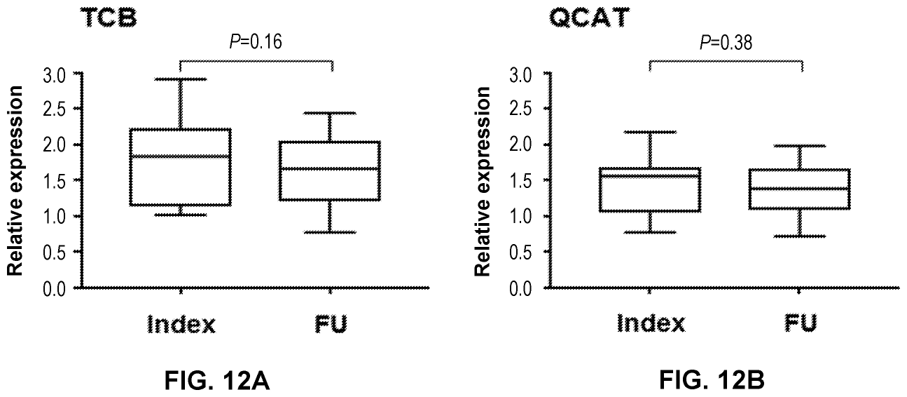

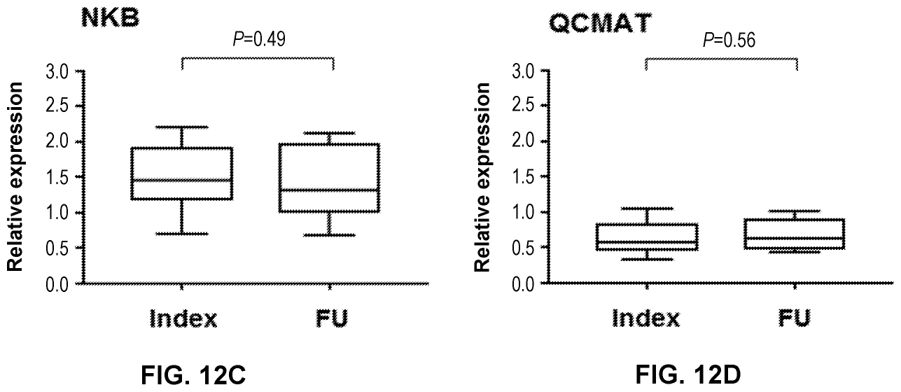

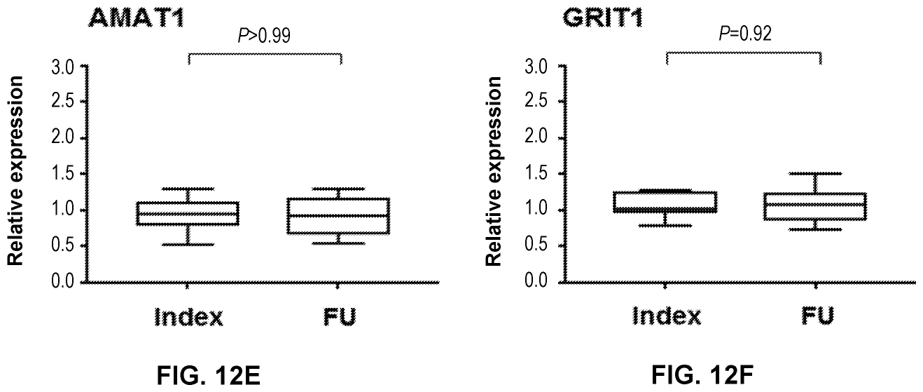

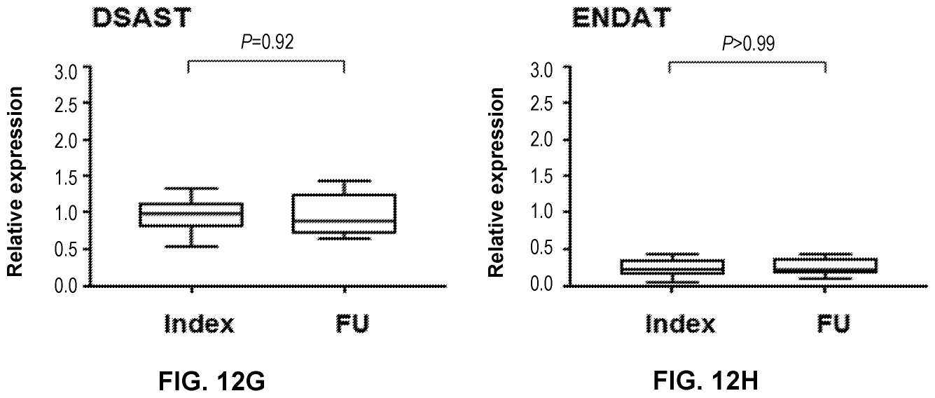

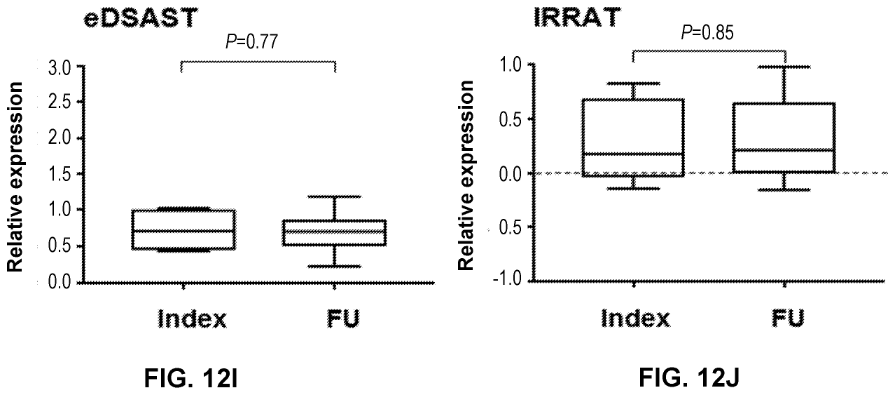

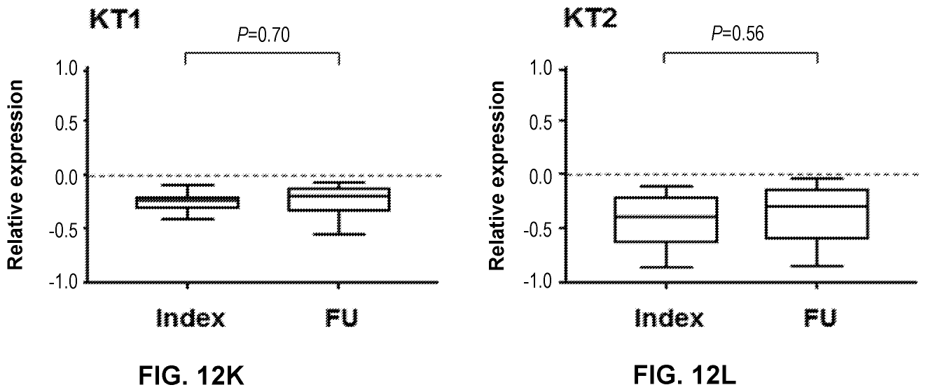

Impact of BIVV009 on histomorphology and molecular biopsy results: As shown in FIG. 11B, there was no change in the extent of microcirculation inflammation [g+ptc score: 4 (IQR: 4-5) in index vs. 5 (3-5) in follow-up biopsies; p >0.99]. As shown in FIG. 11C, transplant glomerulopathy [cg score: 2 (1-3) vs. 2 (1-3); p=0.38] remained unchanged. There was no significant change in molecular classifiers related to rejection: ABMR score: 0.78 (0.49-0.96) vs. 0.82 (0.58-0.96); p=0.67 (FIG. 1D); all Rejection score: 0.75 (0.62-0.85) vs. 0.71 (0.59-0.87); p>0.99 (FIG. 11E); TCMR score: 0.01 (0.0-0.03) vs. 0.02 (0.0-0.01); p=0.44 (FIG. 11F)], acute kidney injury [AKI score: 0.19 (0.02-0.67) vs. 0.24 (0.11-0.57); p=0.57 (FIG. 11G)], or chronic injury (Atrophy/Fibrosis score: 0.41 (0.31-0.69) vs. 0.37 (0.16-0.51); p=0.43 (FIG. 11H). To evaluate the impact of CP blockade on various transcript subsets with distinct molecular pathogenesis-associated annotation, we compared—as illustrated in FIG. 12A-12L—changes in selected PBT scores. Comparing index with follow-up biopsies, we found no significant differences (FIG. 12A-12L).

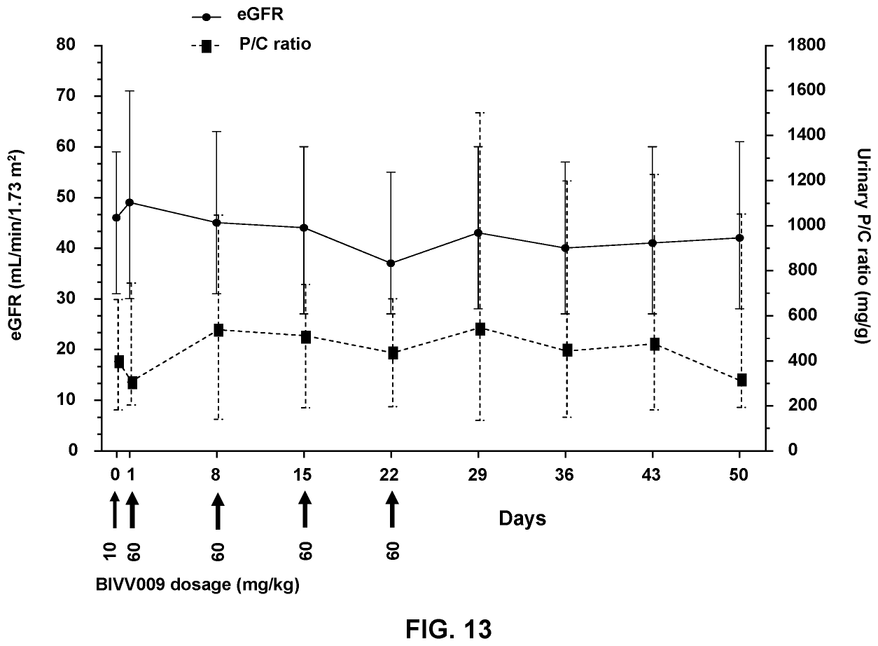

Kidney function and safety outcomes: As illustrated in FIG. 13, there was no change in median eGFR[46 (IQR:27-61) vs. 42 (27-65); p=0.85] and protein/creatinine ratio [399 (IQR: 181-672) vs. 310 (141-1,222); p=0.88] from baseline to day 50. Adverse events recorded during the study period are provided in Table 4.

TABLE 4
Adverse Events

| Parameter | Study cohort (N = 10) |
|---|---|
| Number of patients with one or more AE | 10 |
| Mild AE | 6 |
| Moderate AE | 4 |
| SAE | 0 |
| AE considered to be related to trial treatment | 0 |
| Individual events, number of patients | |
| Headache | 3 |
| Peripheral edema | 3 |
| Fatigue | 2 |
| Asthenia | 1 |
| Muscle spasms | 1 |
| Pain in extremity | 1 |
| Arthralgia | 1 |
| Vomiting | 1 |
| Rhinitis | 1 |
| Procedural pain | 1 |
| Dyspnea | 1 |
| Arterial hypertension | 1 |
| CMV viremia | 1 |

AE, adverse event;
SAE, severe adverse event

Treatment was tolerated well. While all subjects had one or more AE, severe adverse events (SAE) did not occur. Six patients had mild and four moderate AE. The most frequent events documented during the study period were headache (n=3), peripheral edema (n=3) and fatigue (n=2). None of the AE were considered to be related to treatment. While there was no case of bacterial or fungal infection, one recipient developed CMV viremia (maximum of 3000 copies/mL) without clinical symptoms six weeks after study initiation, which was reversible under a course of oral valgancyclovir.

These results show that BIVV009 is able to block the CP, both in serum and in the tissue.

### Example 3

### The Safety, Tolerability, and Pharmacokinetics & Pharmacodynamics of Multiple-Dose BIVV009 in Patients With Chronic Immune Thrombocytopenia (ITP)

This example provides a humanized anti-C1s antibody, BIVV009 (also known as TNT009), that provides clinical benefit for patients with chronic immune thrombocytopenia (ITP). The purpose of this Phase 1 study is to explore the safety, preliminary clinical benefit, and activity of BIVV009 in patients with chronic immune thrombocytopenia. The study will be an interventional study comprising a single group assignment. Ten patients will be enrolled in the study.

TABLE 5
Arms and Interventions

| Arms | Assigned Interventions |
|---|---|
| Experimental: BIVV009 | Drug: BIVV009 |
| Participants will receive a fixed dose | BIVV009 5.5 grams as IV |
| intravenous (IV) infusion of 5.5 grams | infusion over approximately |
| BIVV009 over approximately 60 minutes | 60 minutes |
| on Days 0, 7, 21, 35 and 49. | |

### Outcome Measures

### Primary Outcome Measure:

1. Incidence of Treatment-Emergent Adverse Events. An AE is any untoward medical occurrence in a participant participating in a clinical study that does not necessarily have a causal relationship with the pharmaceutical/biological agent under study. A serious adverse event (SAE) is any AE that results in: death, persistent or significant disability/incapacity, requires inpatient hospitalization or prolongation of existing hospitalization, is life-threatening experience, is a congenital anomaly/birth defect and can jeopardize participant and/or can require medical or surgical intervention to prevent one of the outcomes listed above. [Time Frame: Time from first dose to the final study visit, assessed up to approximately 13 weeks].

2. Number of Participants With Premature Study Terminations. Number of participants with premature study terminations will be assessed. [Time Frame: Up to Day 91].

3. Number of Participants With Clinical Laboratory Abnormalities. Clinical laboratory abnormalities included hematology, clinical chemistry panel, coagulation safety panel, urinalysis, and antibodies against platelet antigens. [Time Frame: Up to Day 91].

### Secondary Outcome Measure:

4. Percentage of Participants With Complete response (CR). Complete response per Evidence Practice Guidelines for Immune Thrombocytopenia: a platelet count greater than or equal to (>=) 100*10\hat{ }9 per liter measured on 2 occasions at least 7 days apart and the absence of bleeding. [Time Frame: Baseline to end of treatment (9 weeks)]

5. Percentage of Participants With Response (R). Response. A platelet count >=30*10\hat{ }9 per liter and a greater than 2-fold increase from baseline measured on 2 occasions at least 7 days apart and the absence of bleeding. [Time Frame: Baseline to end of treatment (9 weeks)]

6. Percentage of Participants With No Response (NR). No response (NR): A platelet count less than (<) 30*10\hat{ }9 per liter, or a less than two-fold increase from baseline, or the presence of bleeding. Platelet count must be measured on 2 occasions more than 1 day apart. [Time Frame: Baseline to end of treatment (9 weeks)]

7. Percentage of Participants With Loss of Complete Response. Loss of complete response: A platelet count <100*10\hat{ }9 per liter measured on 2 occasions more than 1 day apart and/or the presence of bleeding. [Time Frame: Baseline to end of treatment (9 weeks)]

8. Percentage of Participants With Loss of Response. Loss of response: A platelet count <30*10\hat{ }9 per liter, or a less than 2-fold increase in platelet count from baseline, or the presence of bleeding. Platelet count must be measured on 2 occasions more than 1 day apart. [Time Frame: Baseline to end of treatment (9 weeks)]

9. Plasma Concentrations of BIVV009. Plasma concentrations of BIVV009 will be assessed. [Time Frame: Up to Day 91]

10. Maximum Observed Plasma Concentration (Cmax) of BIVV009 Maximum observed concentration of BIVV009 in plasma will be assessed. [Time Frame: Up to Day 91]

11. Time to Reach Maximum Observed Plasma Concentration (Tmax) of BIVV009 Time to Reach Maximum Observed Plasma Concentration (Tmax) of BIVV009 will be assessed. [Time Frame: Up to Day 91]

12. Area Under the Concentration-time Curve (AUC) From Hour 0 Over the Dosing Interval (AUC[0-tau]) of BIVV009. Area Under the Concentration-time Curve (AUC) From Hour 0 Over the Dosing Interval (AUC[0-tau]) of BIVV009 will be assessed. [Time Frame: Up to Day 91]

13. Number of Participants With Anti-drug antibodies (ADAs) Against BIVV009. Blood samples will be collected to determine number of participants with anti-drug antibodies (ADAs) against BIVV009. [Time Frame: Up to Day 91]

14. Complement System Classical Pathway Levels as Measured by WIESLAB® Assay. Inhibition by BIVV009 of the complement system classical pathway measured by the WIESLAB® assay. [Time Frame: Up to Day 91]

15. Total Complement (CH50) Levels. Complement CH50 is a blood test that helps us determine whether protein abnormalities and deficiencies in the complement system are responsible for any increase in autoimmune activity. It will be assessed using complement assays. [Time Frame: Up to Day 91].

16. Total Complement Factor C4 Levels. Total C4 Levels will be assessed in plasma using complement assays. [Time Frame: Up to Day 91].

17. C1 Complex Components: C1q and C1s Levels. C1q and C1s Levels will be assessed in plasma using complement assays. [Time Frame: Up to Day 91].

18. Number of Participants With Autoantibodies Against Platelet Antigens (GPIIb/IIIa and GPIb/IX). Autoantibodies against platelet antigens (GPIIb/IIIa and GPIb/IX) will be assessed. [Time Frame: Up to Day 91].

### Eligibility

### Inclusion Criteria:

Chronic ITP refractory to standard therapy as defined by a platelet count of <30*10\hat{ }9 per liter in participants who have lack of response to at least two of the following ITP treatments: corticosteroids, rituximab, thrombopoietin agonists, azathioprine, danazol, cyclosporin A, or mycophenolate mofetil.
Normal prothrombin time (PT/INR) and activated partial thromboplastin time (aPTT).
No history of a coagulation disorder.
Hemoglobin level greater than (>) 10 gram per deciliter (g/dL) (following blood transfusion is acceptable) and normal white blood cell (WBC) and neutrophil counts (elevated WBC/absolute neutrophil count [ANC] attributed to steroid treatment is acceptable).
Eastern Cooperative Oncology Group (ECOG) performance status grade less than or equal to (=) 2.
Previously vaccinated against encapsulated bacterial pathogens (Neisseria meningitidis, Meningitis B, Haemophilus influenzae, and Streptococcus pneumoniae) or willing to undergo vaccination. Reimmunization with meningococcal conjugate is required if the last vaccination was >5 years prior to enrollment.
Adequate intravenous (IV) access.

### Exclusion Criteria:

Clinically significant medical history or ongoing chronic illness that would jeopardize the safety of the participant or compromise the quality of the data derived from his/her participation in this study.
Clinically relevant infection of any kind within the preceding month of enrollment.
History of venous or arterial thrombosis within the preceding year of enrollment.
Use of aspirin, nonsteroidal anti-inflammatory drugs (NSAIDs), or anticoagulants within 1 week of enrollment.
History of lupus or other autoimmune disorder associated with antinuclear antibodies (ANAs) at screening.
Secondary immune thrombocytopenia from any cause including lymphoma, chronic lymphocytic leukemia, and drug-induced thrombocytopenia.
Positive hepatitis panel (including hepatitis B surface antigen and/or hepatitis C virus antibody) prior to or at Screening.
Positive human immunodeficiency virus (HIV) antibody prior to or at Screening.

### Example 4

### A Randomized, First-in-Human, Healthy Volunteer Trial of BIVV009, a Humanized Antibody for the Specific Inhibition of the Classical Complement Pathway

This example provides a first-in-human, double-blind, randomized, placebo-controlled, dose-escalation trial of BIVV009 in healthy adults.

Healthy female and male subjects aged ≥18 years were eligible for enrollment. Subjects had to be either previously vaccinated against encapsulated bacterial pathogens (Neisseria meningitidis, Haemophilus influenzae, and Streptococcus pneumoniae) or willing to undergo vaccination (at least 14 days before study drug administration). Subjects with body weight >98 kg (for all subjects in all dose cohorts other than the 100 mg/kg dose cohort of part A, for which the body weight upper limit was 58 kg) were excluded.

Trial Design: In part A, single doses of BIVV009 (0.3, 1, 3, 10, 30, 60, or 100 mg/kg) or placebo were infused intravenously over a period of approximately 60 minutes in a 3:1 ratio (0.3 and 1 mg/kg: n=4 per group; the remainder: n=8 per group). The lowest dose of BIVV009 given was based on 1/300^{th} of the No Observed Adverse Effect Level (NOAEL) in non-human primates (NHP), which was expected not to inhibit the classical pathway. In part B, four repeated doses of BIVV009 (30 or 60 mg/kg) or placebo were given once weekly to 16 subjects (8 per dose group) in a 3:1 ratio, with an additional observation period of 2 weeks. Infusion of BIVV009 or placebo followed a stepwise dose-escalation procedure. Part B was initiated after confirming the tolerability and safety of the highest dose step of part A. In part A, safety (adverse events, vital signs), pharmacokinetic (PK) profiles and pharmacodynamic (PD) responses were monitored 1 h before and 0.5, 1, 4, 8 and 24 h after the start of the infusion and 2, 3, 4, 7, and 14 days after administration. In part B, safety, PK and PD were monitored at the following timepoints: 1 h before and 0.5, 1, 4 and 8 h after the start of the first infusion, and daily on the next 4 days; 1 h before and 4 h after the start of the second and third infusion; 1 h before and 0.5, 1, 4 and 8 h after the start of the last/fourth infusion, and daily on the next 4 days; 1 week and 2 weeks after the last/fourth infusion.

Pharmacokinetics (PK): Pharmacokinetic variables of BIVV009 were determined from serum concentrations and included maximum concentration (Cₘₐₓ), half-life (t_{1/2}), time to reach maximum concentration (tₘₐₓ), area under the concentration-time curve (AUC) up to the last time point with a concentration above the lower limit of quantification (AUC_{∞}), and up to the last time point with a concentration above the lower limit of quantification extrapolated to infinity (AUCₗₐₛₜ). Serum concentrations of BIVV009 were measured with a validated immunoassay by a GLP-certified laboratory (Vela Laboratories, Vienna, Austria).

Pharmacodynamics (PD): Activity of the classical complement pathway was measured semi-quantitatively in serum by the use of a commercially available enzyme immunoassay (Complement System Classical Pathway WIESLAB; Euro Diagnostica AB, Malmö, Sweden) as previously published (Roos, A. & Wieslander, J., Methods Mol. Biol. 1100:11-23 (2014)).

Pharmacokinetics/Pharmacodynamics (PK/PD): The relationship between concentrations of BIVV009 and CP activity was first explored to assess potential delay in response (i.e., hysteresis). Based on exploratory analyses, the concentration-effect relationship of BIVV009 and CP activity was explored using various PK/PD models. PK/PD modeling was performed with Phoenix NLME (V7).

Safety: Safety measurements were assessed by adverse events, vital signs, physical examination, electrocardiogram, and laboratory tests. The severity of adverse events was graded using the National Cancer Institute Common Terminology Criteria for Adverse Events (CTCAE, v4.03). Laboratory tests were determined in an accredited routine laboratory and consisted of hematology, blood chemistry and coagulation tests, urinalysis, and immunoassays for systemic lupus erythematosus (SLE) associated autoantibodies.

Immunogenicity: BIVV009 antibodies (anti-drug antibodies [ADAs]) were analyzed in a two-step approach (screening assay, followed by a confirmatory assay and absolute ADA concentration determination) with validated immunoassays by a GLP-certified laboratory (Vela Laboratories, Vienna, Austria). ADAs were measured in serum before infusion and after 7 and 14 days in part A, and before infusion and after 7, 21 and 35 days in part B.

Sample size and statistical analysis: No formal sample size calculation was conducted, but the clinical trial followed the usual dose escalation design for first-in-human trials. The first two cohorts included only three subjects because it was assumed that only minimal PK/PD readouts could be obtained in those groups. No inferential statistical testing was performed because no formal hypothesis was tested. Data are presented descriptively, as appropriate.

### Results

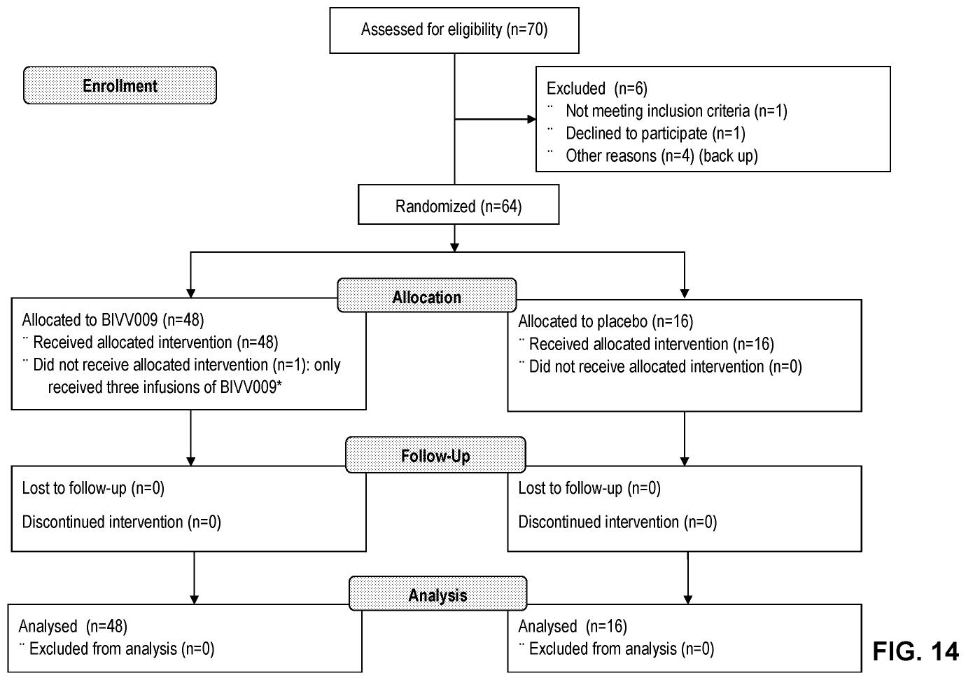

Healthy female and male subjects aged 19 to 59 years (mean age placebo: 33.9, BIVV009: 31.7) were included between Jun. 29, and Dec. 10, 2015. A flow diagram of the progress through the phase-1 trial is shown in FIG. 14. All other subjects randomized in part B received four doses BIVV009 as scheduled.

TABLE 6
Summary statistics for BIVV009 serum pharmacokinetic parameters by treatment.

| | | Cₘₐₓ | | tₘₐₓ | | AUC_{0-∞} | | AUC₀₋₁₆₈ | | half-life | |
|---|---|---|---|---|---|---|---|---|---|---|---|
| | | (μg/mL) | | (h) | | (μg · h/mL) | | (μg · h/mL) | | (h) | |
| Part A | | | | | | | | | | | |
| single BIVV009 | | | | | | | | | | | |
| (mg/kg, 60 min | | | | | | | | | | | |
| infusion) | | | | | | | | | | | |
| 3 | (N = 6) | 40 | (28) | 2.5 | (1, 8) | NC | | 521 | (57) | NC | |
| 10 | (N = 6) | 211 | (21) | 4 | (1, 8) | 7330 | (32) | 6368 | (28) | 19.1 | (37.8) |
| 30 | (N = 6) | 602 | (14) | 2.5 | (1, 24) | 55168 | (27) | 48795 | (22) | 53.3 | (19.8) |
| 60 | (N = 6) | 1464 | (16) | 1.0 | (1.0, 8.0) | 162835 | (13) | 124342 | (12) | 65.1 | (31.7) |
| 100 | (N = 6) | 2036 | (14) | 1.0 | (1.0, 23.5) | 335927 | (8) | 198026 | (11) | 132 | (18.5) |
| Part B | | | | | | | | | | | |
| single BIVV009 | | | | | | | | | | | |
| (mg/kg, 60 min | | | | | | | | | | | |
| infusion) | | | | | | | | | | | |
| 30 | (N = 6) | 653 | (16) | 2.5 | (1, 4) | 52161 | (15) | 46604 | (12) | 51.2 | (15.9) |
| 60 | (N = 6) | 1252 | (17) | 6 | (1, 8) | 150570 | (13) | 111480 | (14) | 87.8 | (14.1) |
| multiple BIVV009 | | | | | | | | | | | |
| (mg/kg, 60 min | | | | | | | | | | | |
| infusion) | | | | | | | | | | | |
| 30 | (N = 6) | 832 | (18) | 6.8 | (4, 8) | 99015 | (30) | 74064 | (19) | 67 | (47) |
| 60 | (N = 6) | 2073 | (10) | 4 | (1, 8) | 557551 | (23) | 235612 | (16) | 210 | (13) |
| 60 | (N = 5)* | 2079 | (11) | 4 | (1, 8) | 568045 | (25) | 237821 | (17) | 212 | (14) |

Values are represented as mean (CV %) for each parameter, except for tmax, for which the values are the median and (minimum-maximum). AUC, area under the concentration-time curve; Cmax, maximum serum concentration; tmax, time to maximum serum concentration; NC, not calculated.
*adjusted for one subject that did not receive the second infusion due to gastroenteritis.

Safety: A total of forty-eight subjects received intravenous infusions of BIVV009, with single doses as high as 100 mg/kg and with four repeated doses given weekly as high as 60 mg/kg. No drug-related serious adverse events, premature withdrawals due to adverse events, or severe drug-related adverse events were observed. In part A, eleven subjects (310%) receiving BIVV009 had a total of eighteen adverse events and six subjects (50%) in the placebo group had a total of eight adverse events. In part B, eight subjects (670%) receiving BIVV009 had a total of nineteen adverse events and all four subjects in the placebo group had a total of ten adverse events. Headache (6/48 subjects=130%) and nasopharyngitis (4/48 subjects=80%) were the most common adverse events reported in subjects receiving BIVV009. Due to the reported association of genetic deficiencies in classical pathway activity and an increased risk of SLE, plasma levels of anti-dsDNA, anti-ANA, and circulating immune complexes were measured throughout the course of the trial. No consistent or meaningful changes in these analytes were observed in subjects dosed with BIVV009. Furthermore, there were no incident cases of SLE or systemic bacterial infection.

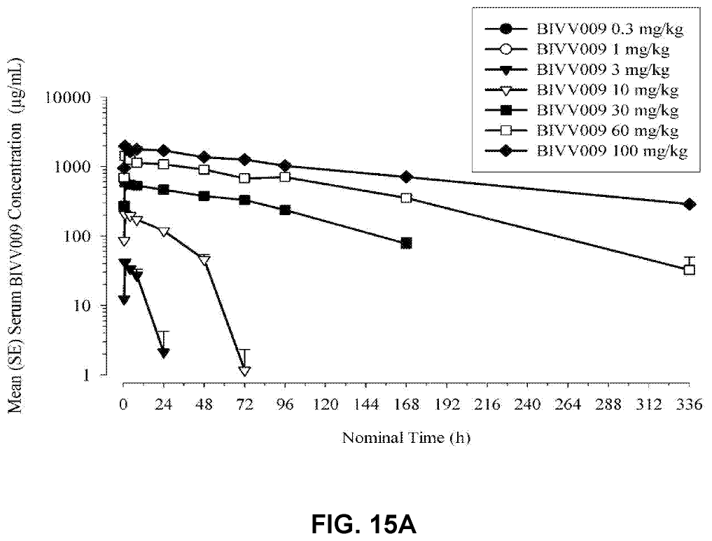

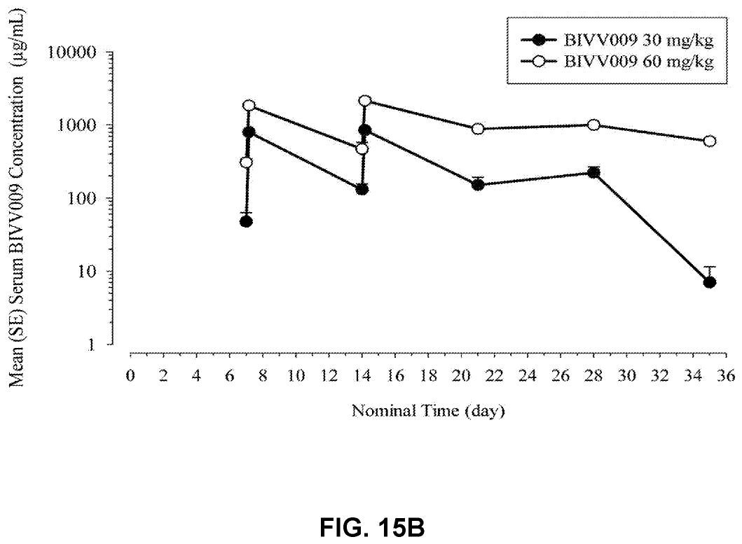

Pharmacokinetics: In part A, intravenous infusions of BIVV009 over 60 minutes were associated with similar median tₘₐₓ values across doses of 3 to 30 mg/kg, with values ranging from 2.5 to 4 h (Table 8). Peak serum BIVV009 concentrations after doses of 60 and 100 mg/kg were observed with the end of infusion (median tₘₐₓ 1 h). Mean Cₘₐₓ increased dose proportionally, ranging from 40 μg/mL to 2036 μg/mL. Over the 10 to 100 mg/kg dose range, mean t_{1/2} ranged from 19 to 132 h and increased with higher dose levels. From 3 to 10 mg/kg and from 10 to 30 mg/kg, the mean BIVV009 exposure (AUC₀₋₁₆₈) increased in a greater than dose proportional manner (12.2- and 7.7-fold, respectively). On the other hand, from 30 to 60 mg/kg and from 60 to 100 mg/kg, the mean BIVV009 exposure (AUC₀₋₁₆₈) increased in a dose proportional manner (2.5- and 1.6-fold, respectively). Serum BIVV009 concentrations were below the limit of quantification with the two lowest doses (0.3 and 1 mg/kg). Mean concentration-time profiles of BIVV009 are provided in FIG. 15A. Based on visual investigation of the concentration-time profiles of BIVV009, non-linear elimination was clearly apparent at concentrations lower than approximately 100 μg/mL.

In part B, peak serum BIVV009 concentrations were observed at a median of 2.5 and 6 h after a single infusion (30 or 60 mg/kg, respectively) and 6.8 and 4 h (30 or 60 mg/kg, respectively) following multiple infusions. For a 2-fold increase in BIVV009 dose from 30 to 60 mg/kg, mean AUC₀₋₁₆₈ increased 2.4-fold after a single infusion and 3.2-fold after multiple infusions. Cₘₐₓ increased 1.9-fold after a single infusion and 2.5-fold after multiple infusions. Mean t_{1/2} ranged from 51.2 to 87.8 h (single 30 or 60 mg/kg dose, respectively) and from 67 to 210 h (multiple 30 or 60 mg/kg doses, respectively). Pre-dose concentrations increased over time, indicating some BIVV009 accumulation (FIG. 15B).

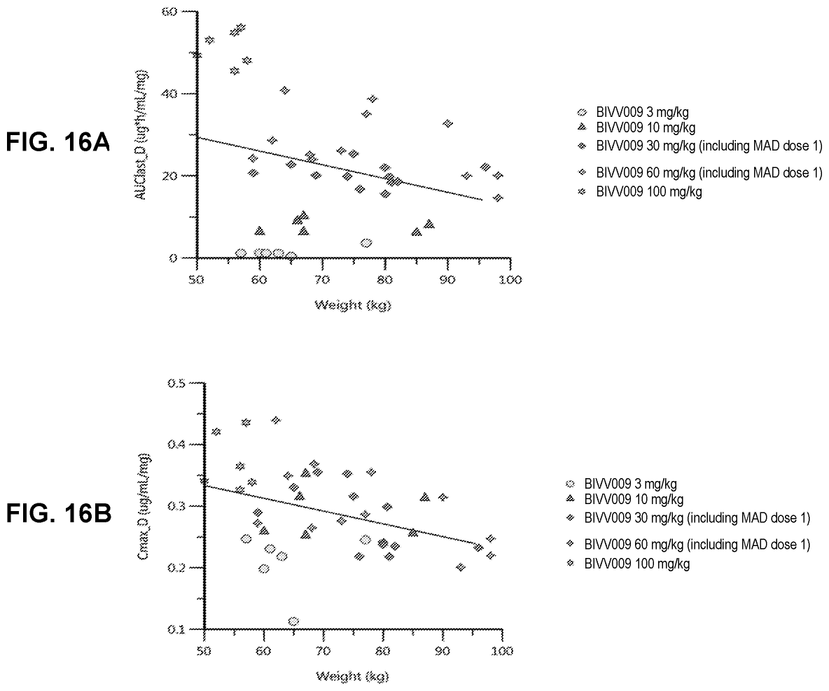

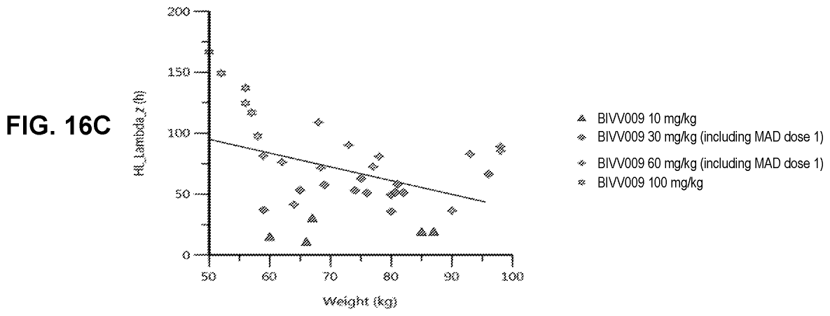

Figures of PK parameters vs individual body weight from part A and part B (first dose) were plotted to explore any potential relationships (FIGS. 16A-16C). For AUCₗₐₛₜ, Cₘₐₓ and half-life vs weight plots, the linear regression line has a negative slope, suggesting that body weight can play a role in BIVV009 exposure and disposition. However, a formal covariate analysis testing the effect of weight on exposure was not performed, since performing such an analysis using the limited number of subjects available in part A and B (i.e. <50 subjects) would be deemed unreliable (see Bonate, P. L. Pharmacokinetic-pharmacodynamic modeling and simulation (Springer: New York, 2006)). These plots should thus be considered exploratory, and further investigation of the dose effect at extremes of the weight range can be warranted.

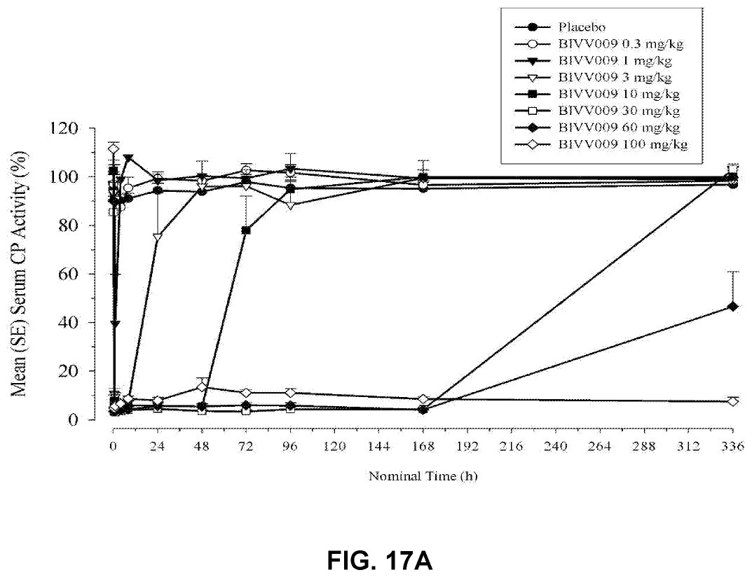

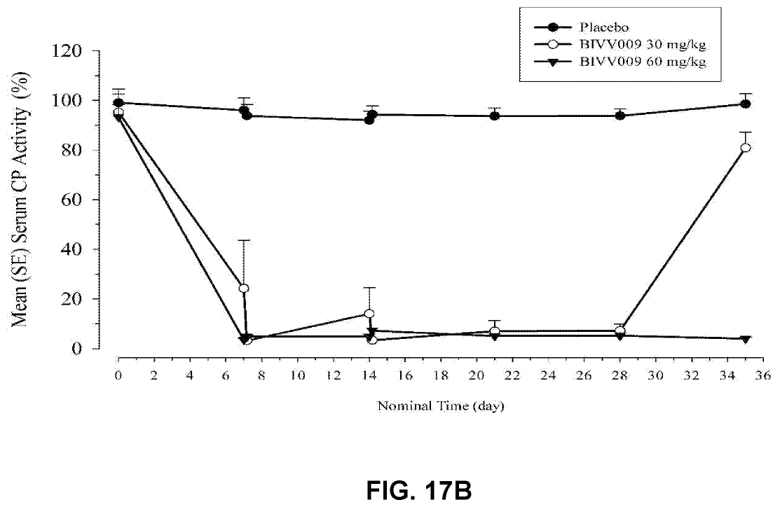

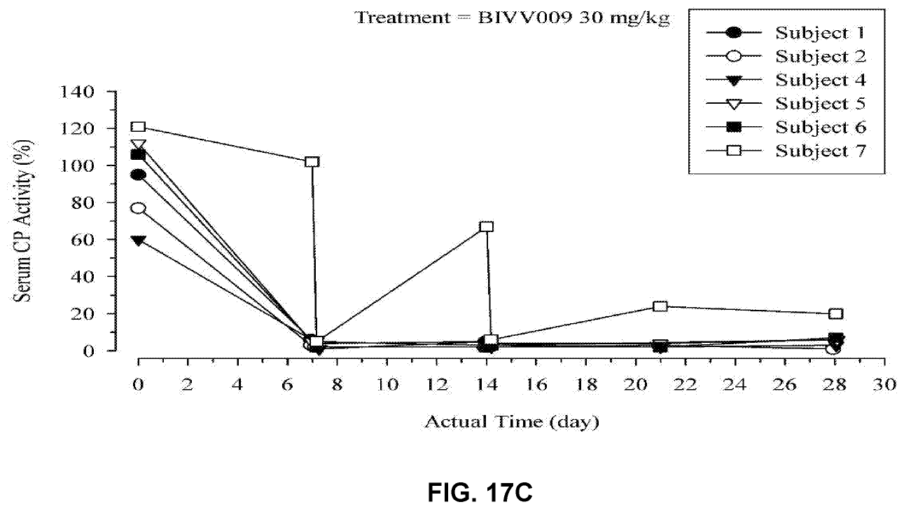

Pharmacodynamics: In part A, all subjects had normal CP activity at baseline (placebo 95%±10%; BIVV009 97%±14%). A single infusion of 3, 10, 30, 60 and 100 mg/kg BIVV009 suppressed CP activity by >90% within 1 hour after start of the infusion (FIG. 17A). The duration of suppression persisted dose-proportionally from 8 h (3 mg/kg) to up to 14 days (100 mg/kg). The CP activity returned to baseline levels within 2 weeks, whereas no reversal was observed in the 100 mg/kg dose group and only a partial return was observed in the 60 mg/kg group.

In part B, all subjects had normal CP activity at baseline (placebo 99%±7%; BIVV009 94%±18%). A single infusion of BIVV009 (30 and 60 mg/kg) profoundly suppressed CP activity by >95% in less than 1 hour after start of the infusion. Multiple infusions suppressed CP activity by ≥90% in almost all individuals (FIG. 17B). Classical pathway activity did not completely return to baseline in the 30 mg/kg BIVV009 dose group 2 weeks after the last infusion (FIG. 17B). At the same time, mean CP activity was still <5% in the 60 mg/kg dose group. Among subjects receiving 30 mg/kg, one individual had pre-dose activity levels of 102, 67 and 24 percent after 7, 14 and 21 days respectively, indicating faster CP activity reversal than the other individuals. However, BIVV009 rapidly suppressed CP activity (<1 h) by >95% compared with baseline in this subject and sustained suppression for at least 5 days (FIG. 17C).

Pharmacokinetic/Pharmacodynamic Correlations of BIVV009 and CP Activity: Based on exploratory analyses, near-maximal CP activity knockdown was observed for both dose levels in part B, much like the knockdown seen at similar dose levels in part A and B (Day 0) and no delay was observed in PD (results retained on file). As a result, individual serum concentrations of BIVV009 and CP activity were time-matched and the PK/PD relationship was modeled using an inhibitory Eₘₐₓ model as described below.

$$E = {E}_{0} - \frac{{I}_{max} \times {C}^{H}}{{C}^{H} + {IC}_{50}^{H}}$$

Where E₀ is the baseline, Iₘₐₓ is the maximum inhibition, C is the concentration of BIVV009, IC₅₀ is the concentration associated to 50% of the maximum effect and H is the Hill factor (also referred as gamma, a parameter used to described sigmoidicity). The relationship between serum BIVV009 concentrations and CP activity is presented in FIG. 3A, while parameters derived with the inhibitory Eₘₐₓ model are presented in Table 9. A steep concentration-effect relationship was observed for the knockdown of serum CP activity. Based on the inhibitory Eₘₐₓ model, the maximum percent inhibition (Iₘₐₓ) of BIVV009 on CP activity was 90.2%, with a 50% knockdown of CP activity predicted at a BIVV009 concentration of 6.2 μg/mL. The BIVV009 concentration associated to a 90% reduction of CP activity (IC₉₀) was 15.5 μg/mL. The very low IC₅₀, combined with a Hill parameter of 2.4 suggests a very steep concentration-effect relationship and that BIVV009 concentrations above 100 μg/mL would be sufficient to maintain a near-maximal knockdown of CP activity and avoid nonlinear PK.

TABLE 7
PK/PD parameters of BIVV009 and CP activity - parts A and B.

| Parameter | Estimate (RSE %) | |
|---|---|---|
| Iₘₐₓ (%) | 90.2 | (1.1) |
| IC₅₀ (μg/mL) | 6.2 | (27.5) |
| E₀ (%) | 94.8 | (1.1) |
| H | 2.4 | (19.9) |

Iₘₐₓ: the maximum inhibition, IC₅₀: concentration associated to 50% of the maximum effect, E₀: baseline value, H: Hill factor.

Immunogenicity: In part A, there were eight subjects (17%), ten subjects (21%), and eighteen subjects (38%) with samples that tested positive in the screening assay at day 0, at day 7, and at day 14, respectively. Confirmatory assays were performed and absolute ADA concentrations were determined for the subjects tested as reactive in the screening assays. At day 7, there was one subject with a confirmed, reactive ADA result (42 ng/mL), which subject had also a reactive but unconfirmed ADA result prior to the first dose of BIVV009. At day 14 there was another subject with a confirmed, reactive ADA result (28 ng/mL).

In part B, there were two subjects (13%), two subjects (13%), one subject (7%), and four subjects (27%) with reactive ADAs at day 0, day 7, day 21, and at day 35, respectively. Antidrug antibodies were positive in 1 of 4 subjects (25%) receiving placebo and in 4 of 12 subjects (33%) receiving BIVV009, all of whom received 30 mg/kg BIVV009. There was one subject with a confirmed ADA, who had a reactive and confirmed ADA result already at day 0 (0 ng/mL), before receiving the first dose of BIVV009.

### DISCUSSION

In this first-in-human trial, the primary objective was to characterize the safety/tolerability profile of BIVV009 in healthy volunteers. Infusions of up to 100 mg/kg of BIVV009 to forty-eight subjects were well tolerated and no serious or severe adverse events occurred. Importantly, although complement inhibition increases the risk of invasive bacterial infection (see Dmytrijuk, A. et al., Oncologist 13:993-1000 (2008)), no systemic bacterial infections were observed during the entire study period, presumably because the mode of action of BIVV009 leaves the alternative pathway and the lectin pathway function intact, and all participants were vaccinated against encapsulated bacterial pathogens prior to dosing. Another theoretical concern could be extrapolated from rare human cases of deficiencies or mutations in classical pathway components, including C1s see (Dmytrijuk, A. et al. (2008)). In this study, no incidental case of SLE was observed in subjects dosed with BIVV009, consistent with the finding that levels of commonly associated serological markers of SLE, including anti-dsDNA, anti-ANA, and circulating immune complexes, were unchanged with BIVV009 treatment. Thus, short term pharmacologic C1s inhibition does not appear to increase the risk of developing SLE. Larger and longer clinical trials will be required to determine this risk under chronic treatment.

The Cₘₐₓ of BIVV009 was dose proportional over the dose range from 10-100 mg/kg. However, the mean BIVV009 exposure (AUC) increased in a greater than dose proportional manner in the lower dose range (3 to 30 mg/kg) and in an approximately dose proportional manner at higher doses (60 to 100 mg/kg). Over the 10 to 100 mg/kg dose range, mean t_{1/2} ranged from 19 to 132 h and increased with higher dose levels. Non-linear elimination of BIVV009 was clearly apparent at concentrations lower than approximately 100 μg/mL. This non-linear behavior suggest potential target-mediated elimination which is usually apparent at lower concentrations, as previously reported for other monoclonal antibodies (see Mould, D. R. & Sweeney, K. R, Curr. Opin. Drug Discov. Devel. 10:84-96 (2007)). As the linear component was more dominant at higher doses, prediction of therapeutic blood concentrations can be more accurate. Following repeated weekly dosing, mean total BIVV009 exposure increased in a slightly greater than dose proportional manner. The trough concentrations also increased with repeated dosing, suggesting some accumulation of BIVV009 in healthy volunteers. BIVV009 doses of 1 mg/kg or lower had little effect on CP activity, whereas complete inhibition, defined by CP activity <10%, was achieved in all subjects who received a BIVV009 dose of 3 mg/kg or higher. The complete inhibition of CP activity persisted <4 days, >7 days and >14 days with low (3, 10 mg/kg), moderate (30, 60 mg/kg) and high (100 mg/kg) doses, respectively. As such, the duration of CP inhibition appears to be dose-related. The data also show that BIVV009 inhibits CP activity at very low doses, even though for a short time. An inhibitory Iₘₐₓ model confirmed a very steep concentration-effect relationship (Hill parameter of 2.4), while the BIVV009 concentration associated with a 90% reduction of CP activity (IC₉₀) was 15.5 μg/mL. Multiple infusions of 60 mg/kg BIVV009 resulted in complete and consistent suppression of CP activity for more than 14 days after the last infusion. In contrast, CP activity was almost reversed in the 30 mg/kg dose group (81% from baseline) at the same time. Therefore, a 60 mg/kg or higher dose in combination with different dosing intervals can achieve long-acting CP inhibition, possibly more suitable for clinical practice. The mean pre-dose CP activity was slightly higher with weekly 30 mg/kg BIVV009 compared with the weekly 60 mg/kg dose. This was caused by considerably higher trough CP activity in one individual in the 30 mg/kg cohort, who also was reactive in the screening and confirmatory ADA assay at the beginning of part B, before receiving BIVV009. Although the pre-formed ADAs presumably had some effects on the individual's PK and PD profile, 30 mg/kg of BIVV009 was still sufficient to induce a rapid and sustained suppression of CP activity without any clinical manifestation. This can indicate that BIVV009 retains most of its inhibitory activity with no apparent side effects, even if ADAs are present. No other participants were confirmed to have ADAs in part B. In part A, two subjects developed ADAs in response to BIVV009 (10 mg/kg and 60 mg/kg dose group).

While specific tests were not performed to characterize neutralizing activity of ADAs, both individuals had similar CP activity when compared to other subjects in their cohorts and the ADA concentrations (42 ng/mL and 28 ng/mL) were ˜500-1000 fold lower than the drug levels required for PD effect, suggesting no clinically relevant inhibition of BIVV009 functional activity. The ADA positive rate of 6% in part A and 8% in part B is comparable to the ADA frequency reported from other studies with therapeutic antibodies (Baker, M. P. et al., Self Nonself 1:314-22 (2010)). Our trial provides mandatory data on safety, PK and PD of BIVV009, but there are also limitations. It is important to recognize that although patients with BP, AMR, WAIHA or CAD share the same underlying effector pathway, differences in safety, PK and PD can exist. Since the proof of concept should be made in the target population, we used an integrated protocol design that included an ongoing investigation of patients with BP, AMR, WAIHA or CAD (Derhaschnig, U. et al., Orphanet J. Rare Dis. 11:134 (2016)). Overall, BIVV009 had a good safety profile and predictable and consistent PK and PD in healthy volunteers.

### Example 5

### BIVV009 Doses for the Phase 3 Studies

Given the extremely steep PK/PD relationship (all-or-none effect) of BIVV009 observed at concentrations ˜20 μg/mL (IC₉₀) and the rapid clearance due to target-mediated drug disposition (TMDD) observed at concentrations <˜100 μg/mL, the dose and dose regimen of BIVV009 have been tailored to maintain BIVV009 trough levels above 100 μg/mL to prevent breakthrough hemolysis in CAgD patients.

A population PK model of BIVV009 was used to determine the appropriate dosing regimen for the Phase 3 studies in patients with Cold Agglutinin Disease (CAgD). A previously developed population PK model using normal healthy volunteers has been updated to include relevant data from all subjects from Parts A, B and C of Study BIVV009-01, available data from the Named Patient Program (NPP) part of Study BIVV009-01 and the multiple dose data from the BIVV009-02 Study. The influence of covariates, including weight and disease state, on the inter-individual variability in the PK of BIVV009 were explored.

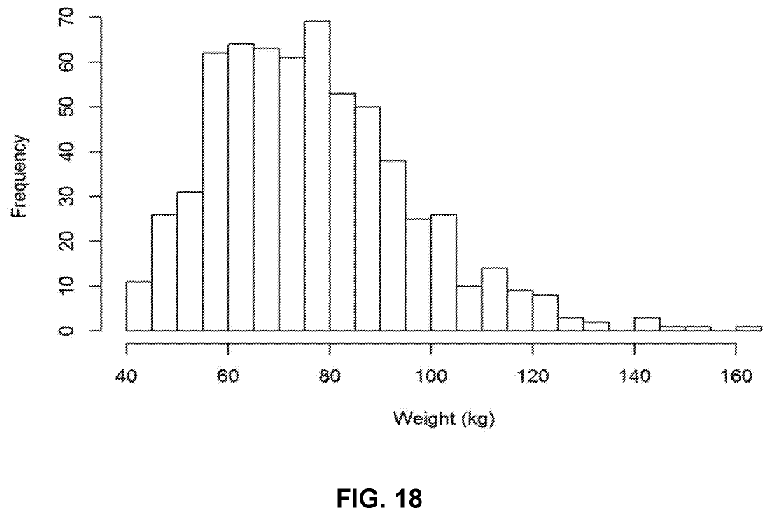

Simulations of select fixed dose, fixed dose combinations and body weight-adjusted dose regimens were performed using the expected weight distribution (FIG. 18) in the Phase 3 studies, and were based on 631 CAgD patients (mean (SD)=77.0 (19.7) kg, median (min−max)=74.8 (40.6−163.3) kg) that were extracted from a US electronic medical record and claims database.

The simulation results indicated that a single fixed dose or a single body weight-adjusted dose would not provide adequate coverage for all subjects (data not shown) with the appropriate safety margins across the expected weight distribution in CAgD patients. Therefore, a tiered flat-dosing approach based on body weight cut-offs is proposed, with a dose of 6.5 g for subjects <75 kg and a dose of 7.5 g for subjects ≥75 kg. Doses are to be administered weekly for the first 2 doses followed by every other week dosing. The weight cut-off of 75 kg was chosen based on the expected weight distribution in CAgD patients with a median weight of 74.8 kg.

The simulations indicate that the proposed dosing regimen is expected to ensure adequate BIVV009 exposures across the weight range to avoid TMDD with approximately 6.2% of the overall patient population predicted to fall below the critical threshold of 100 μg/mL (Table 8).

TABLE 8
Predicted Median Trough Concentration (95% Prediction Interval (PI)) and Proportion
of CAgD Patients with Trough Concentrations Below 100 μg/mL and 20 μg/mL
(n = 50; 200 Replicates) Using Proposed Body Weight Distributions

| | | Median Trough | | | Mean % Below | Mean % Below |
|---|---|---|---|---|---|---|
| | Weight | Concentration | 90% PI | 90% PI | 100 μg/mL | 20 μg/mL |
| Regimen* | Group | (μg/mL) | Lower | Upper | (90% PI) | (90% PI) |
| 6.5 g < 75 kg, | <75 kg | 1260 | 169 | 2920 | 4.1 (0.0-11.5) | 3.2 (0.0-9.5) |
| 7.5 g ≥ 75 kg | ≥75 kg | 665 | 28.1 | 1670 | 8.3 (0.0-18.2) | 4.7 (0.0-11.6) |
| | All | 907 | 55.8 | 2500 | 6.2 (2.0-12.0) | 4.0 (0.0-8.0) |

*Dosing frequency: Weekly x 2, followed by every other week dosing.

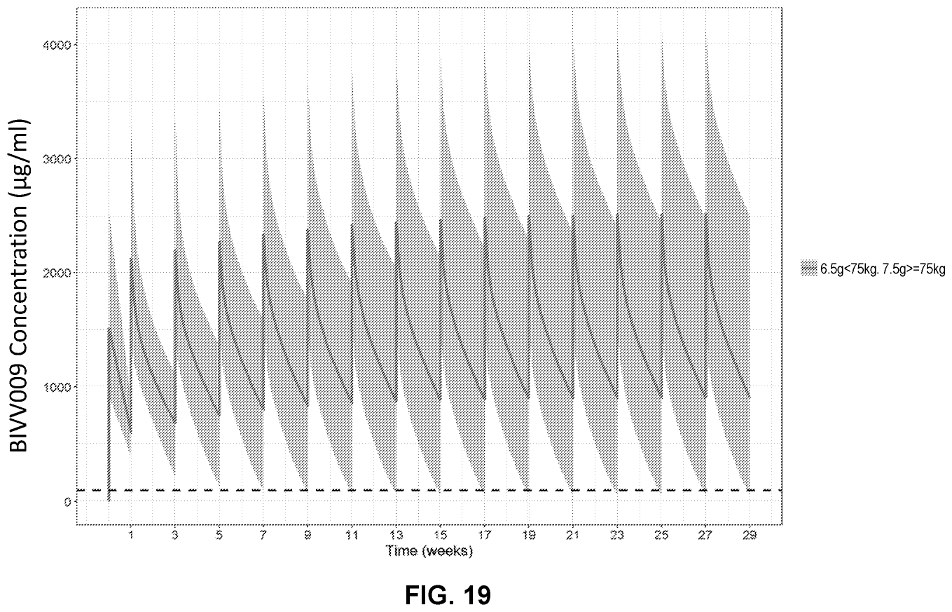

The percentage of subjects at risk for TMDD (i.e., BIVV009 concentrations <100 g/mL) were 4.1% for subjects <75 kg and were slightly higher at 8.3% for subjects ≥75 kg. Only 4% of the overall patient population will fall below the 20 μg/mL threshold needed to maintain efficacy. FIG. 19 shows the simulated concentration vs. time profiles (median and 90% prediction intervals (PI)) for the proposed dosing regimen in CAgD patients.

The exposures estimated for the proposed dosing regimen maintain adequate safety margins with respect to the 6 month GLP cynomolgus monkey studies that identified a NOAEL of 180 mg/kg weekly. Based on the predicted exposures for the proposed dosing regimen, the Cmax and AUC at steady state have an approximately 4-fold safety margin over the chronic toxicology study (Table 9).

TABLE 9
Safety Margins for Cₘₐₓ and AUC at Steady State
in Relation to the NOAEL in Cynomolgus Monkeys.

| | Cynomolgus Monkey | | Simulated CAgD patients | | | | Safety |
|---|---|---|---|---|---|---|---|
| Dose | 180 mg/kg weekly | | Tiered Dosing* | | | | Margin |
| Cmax at | 11700 | (5380) | <75 | kg | 2830 | (910.4) | 4.13 |
| steady state | | | ≥75 | kg | 2430 | (652.8) | 4.81 |
| (μg/mL) | | | All | | 2630 | (817.7) | 4.45 |
| AUC at | 2060000 | (966000) | <75 | kg | 636000 | (288220.8) | 3.24 |
| steady state | | | ≥75 | kg | 425000 | (172871.3) | 4.85 |

| | | | | |
|---|---|---|---|---|
| (μg · hr/mL) | All | 531000 | (260320.0) | 3.88 |

Cₘₐₓ and AUC at steady state are presented as Mean (SD)
*Dose: 6.5 g for subjects <75 kg; 7.5 g for subjects ≥75 g. Dosing frequency: Weekly x 2, followed by every other week dosing.

In summary, the proposed tiered flat-dosing regimen is predicted to maintain target trough concentrations >100 μg/mL in approximately 94% of CAgD subjects to prevent breakthrough hemolysis while providing sufficient safety margins in relation to the NOAEL exposures seen in the cynomolgus monkeys.

### Example 6

### The Safety, Tolerability, and Efficacy of BIVV009 Administration in Patients With Primary Cold Agglutinin Disease Who have a Recent History of Blood Transfusion

This example provides a pivotal, open-label, multicenter study to assess the efficacy and safety of the humanized anti-C1s esterase antibody (BIVV009) in patients with primary cold agglutinin disease (CAgD) who have a recent history of blood transfusion. The study consists of two parts: Part A and Part B.

The co-primary objectives of this study are (i) to determine whether BIVV009 administration increases hemoglobin (Hgb) levels ≥2 g/dL from baseline or to ≥12 g/dL and obviates the need for blood transfusion during treatment in patients with primary CAgD who have a recent history of transfusion (Part A) and (ii) to evaluate the long-term safety and tolerability of BIVV009 in patients with primary CAgD (Part B).

The secondary objectives for Part A of this study include: (i) to assess the effect of BIVV009 on clinical events and laboratory parameters related to hemolysis and anemia in patients with primary CAgD; (ii) to assess the effect of BIVV009 on quality of life (QOL) in patients with primary CAgD; and (iii) to evaluate the overall safety and tolerability of BIVV009 in patients with primary CAgD. The secondary objective for Part B of this study is to investigate the durability of response during long-term treatment with BIVV009 in patients with primary CAgD.

The exploratory objectives (Part A) include: (i) to assess the effect of BIVV009 on specific complications of CAgD; (ii) to evaluate the effect of BIVV009 on certain disease-related biomarkers in patients with primary CAgD; and (iii) to evaluate the pharmacokinetics of BIVV009.

TABLE 10
Arms and Interventions

| Arms | Assigned Interventions |
|---|---|
| Experimental: BIVV009 | Drug: BIVV009 (18 mg/mL) |
| Part A: | |
| Participants will receive a fixed dose | |
| intravenous (IV) infusion of total of | |
| 6.5-7.5 grams BIVV009 on Days 0, 7, and | |
| every 14 days thereafter through Week 25. | |
| Part B: | |
| Participants from Part A will receive a | |
| fixed dose intravenous (IV) infusion of | |
| 6.5-7.5 grams BIVV009 biweekly starting | |
| at Week 27 and continuing for up to 1 | |
| year. | |

### Endpoint(s)

### Primary Endpoint:

The primary efficacy endpoint is the responder rate. A patient will be considered a responder if he or she did not receive a blood transfusion from Week 5 through Week 26 (EOT) and did not receive treatment for CAgD beyond what is permitted per protocol. Additionally, the patient's Hgb level must meet either of the following criteria: (i) Hgb level is ≥12 g/dL at the treatment assessment endpoint (defined as mean value from Weeks 23, 25, and 26); or (ii) Hgb increased ≥2 g/dL from baseline (defined as the last Hgb value before administration of the first dose of the study drug) at treatment assessment endpoint.

The primary endpoint will be assed at Week 26 (end of treatment).

### Secondary Endpoint(s):

The secondary endpoints include the following:
Mean change from baseline in bilirubin (excluding patients with Gilbert's Syndrome) at the treatment assessment endpoint (mean of values at Week 23, 25, and 26).
Mean change from baseline in QOL, as assessed by the change in Functional Assessment of Chronic Illness Therapy (FACIT)-Fatigue scale scores at the treatment assessment endpoint.
Mean change from baseline in lactate dehydrogenase (LDH) at the treatment assessment endpoint.
Number of transfusions and number of units after the first 5 weeks of study drug administration.
Mean change from baseline in Hgb at the treatment assessment endpoint.

The above secondary endpoints will be assessed at Week 26 (end of treatment).

All patients will receive a standard of care treatment upon the end of their participation in this study.

### Eligibility

### Inclusion Criteria:

Adult males and females ≥18 years of age at screening.
Body weight of ≥39 kg at screening.
Confirmed diagnosis of primary CAgD based on the following criteria: (a) chronic hemolysis; (b) polyspecific direct antiglobulin test (DAT) positive; (c) monospecific DAT strongly positive for C3d; (d) cold agglutinin titer ≥64 at 4° C.; (e) IgG DAT ≤1+; and (f) no overt malignant disease.
History of at least one documented blood transfusion within 6 months of enrollment.
Hemoglobin level ≤10.0 g/dL.
Bilirubin level above the normal reference range.
Ferritin level within the normal reference ranges unless outside normal range and deemed not clinically significant by the Investigator (or designee).
Presence of one or more of the following CAgD-related signs or symptoms within 3 months of screening: (a) symptomatic anemia defined as (i) fatigue, (ii) weakness, (iii) shortness of breath, (iv) palpitations (e.g., fast heart beat), (v) light headedness, and/or (vi) chest pain; (b) acrocyanosis; (c) Raynaud's syndrome; (d) hemoglobinuria; (e) disabling circulatory symptoms; and/or (f) major adverse vascular event (including thrombosis).
Bone marrow biopsy within 6 months of screening with no overt evidence of lymphoproliferative disease or other hematological malignancy. An additional bone marrow biopsy will be required if the prior bone marrow is deemed unsuitable for analysis by the Sponsor.
Vaccinations against encapsulated bacterial pathogens (Neisseria meningitis, Meningitis B, Haemophilus influenza, and Streptococcus pneumoniae) within 5 years of enrollment.

### Exclusion Criteria:

Cold agglutinin syndrome secondary to infection, rheumatologic disease, or active hematologic malignancy.
Clinically relevant infection of any kind within the month preceding enrollment (e.g., active hepatitis C, pneumonia).
Clinical diagnosis of systemic lupus erythematosus (SLE), or other autoimmune disorders with anti-nuclear antibodies at screening.
Positive hepatitis panel (including hepatitis B surface antigen and/or hepatitis C virus antibody) prior to or at screening.
Positive human immunodeficiency virus (HIV) antibody at screening.
Treatment with rituximab monotherapy within 3 months or rituximab combination therapies (e.g., with bendamustine, fludarabine, ibrutinib, or cytotoxic drugs) within 6 months prior to enrollment.
Concurrent treatment with corticosteroids other than a stable daily dose equivalent to ≤10 mg/day prednisone for previous 3 months.
Erythropoietin deficiency. Concurrent treatment with erythropoietin is permitted if the patient has been on a stable dose for the previous 3 months.
Concurrent usage of iron supplementation unless the patient has been on a stable dose for at least 4 weeks.
Clinically significant medical history or ongoing chronic illness that would jeopardize the safety of the patient or compromise the quality of the data derived from his/her participation in this study (as determined by the Investigator [or the designee]) at screening.
Concurrent treatment with other experimental drugs or participation in another clinical trial with any investigational drug within 30 days or 5 half-lives, whichever is greater, prior to treatment start.
Females who are pregnant, lactating, or, if having reproductive potential, are considered potentially unreliable with respect to contraceptive practice.

### Example 7

### The Safety, Tolerability, and Efficacy of BIVV009 Administration in Patients With Primary Cold Agglutinin Disease Without a Recent History of Blood Transfusion

This example provides a randomized, double-blind, placebo-controlled study to assess the efficacy and safety of the humanized anti-C1s esterase antibody (BIVV009) in patients with primary cold agglutinin disease (CAgD) who have not received a recent blood transfusion. This study also consists of two parts: Part A and Part B.

The co-primary objectives of this study are (i) to determine whether BIVV009 administration results in a ≥1.5 g/dL increase in hemoglobin (Hgb) level and avoidance of transfusion in patients with primary CAgD without a recent history of blood transfusion (Part A) and (ii) to evaluate the long-term safety and tolerability of BIVV009 in patients with primary CAgD (part B).

The secondary objectives for Part A of this study include: (i) to assess the effect of BIVV009 on clinical events and laboratory parameters related to hemolysis and anemia in patients with primary CAgD; (ii) to assess the effect of BIVV009 on specific complications of CAgD; and (iii) to assess the effect of BIVV009 on quality of life (QOL) in patients with primary CAgD. The secondary objective for Part B of this study is to investigate the durability of response during long-term treatment with BIVV009 in patients with primary CAgD.

TABLE 11
Arms and Interventions

| Arms | Assigned Interventions |
|---|---|
| Experimental: BIVV009 | Drug: BIVV009 (18 mg/mL) |
| Control: Placebo | or placebo |
| Part A: | |
| Participants will receive a fixed dose | |
| intravenous (IV) infusion of total of | |
| 6.5-7.5 grams BIVV009 on Days 0, 7, and | |
| every 14 days thereafter through Week 25. | |
| Part B: | |
| Participants from Part A will receive a | |
| fixed dose intravenous (IV) infusion of | |
| 6.5-7.5 grams BIVV009 biweekly starting | |
| at Week 27 and continuing for up to 1 | |
| year. | |

### Endpoint(s)

### Primary Endpoint:

The primary efficacy endpoint is the responder rate. A patient will be considered a responder if he or she did not receive a blood transfusion from Week 5 through Week 26 (i.e., end of treatment, EOT) and did not receive treatment for CAgD beyond what is permitted per protocol. Additionally, the patient's Hgb level must meet the following criterion: Hgb increase ≥1.5 g/dL from baseline (defined as the last Hgb value before administration of the first dose of study drug) at treatment assessment endpoint (defined as mean value from Weeks 23, 25, and 26).

The primary endpoint will be assed at Week 26 (end of treatment).

### Secondary Endpoint(s):

The secondary efficacy endpoints for part A of the study include the following:
Mean change from baseline in Hgb at treatment assessment endpoint (mean of values at Weeks 23, 25, and 26).
Mean change from baseline in bilirubin (excluding patients with Gilbert's Syndrome) at treatment assessment endpoint.
Mean change from baseline in QOL, as assessed by the change in Functional Assessment of Chronic Illness Therapy (FACIT)-Fatigue scale scores at the treatment assessment endpoint.
Mean change from baseline in lactate dehydrogenase (LDH) at the treatment assessment endpoint.
Incidence of solicited symptomatic anemia at EOT.

For part B of the study, the following parameters of disease activity will be assessed to determine the secondary efficacy endpoints: Hemoglobin; Bilirubin (total); QOL assessments (FACIT-fatigue, EQ-5D-5L, SF-12, and PGIC scale); LDH; Transfusion requirements; and Haptoglobin._The above secondary endpoints will be assessed at Weeks 23, 25, and 26.

All patients will receive a standard of care treatment upon the end of their participation in this study.

### Eligibility

Inclusion Criteria are similar to the inclusion criteria in Example 6. Additional inclusion criteria are:
Adequate IV access.
If female, must be post-menopausal, surgically sterile, or be established on (≥3 months prior to screening) and agree to continue to use the same highly effective methods of birth control throughout the study and for 6 weeks following administration of the last dose of study drug.
Males must be surgically sterile for at least 90 days or when sexually-active with female partners of child-bearing potential will agree to use highly effective contraception from Day 0 until 6 weeks following administration of the last dose of study drug.
Able to comprehend and give informed consent.
Able to comply with the requirements of the study and to complete the full sequence of protocol-related procedures.
   Exclusion Criteria are the same as the exclusion criteria in Example 6.

### Example 8

### BIVV009 Attenuates Complement Deposition Bullous Pemphigoid Patients

This example shows that an anti-C1s antibody of the present disclosure inhibited or completely abolished C3c and C3d deposition associated with bullous pemphigoid (BP).

The first experiment was performed in vitro. Serum samples from 47 bullous pemphigoid patients were collected. Sera from normal healthy individuals was used as a control. The samples were tested for the presence of circulating IgG autoantibodies (characteristic of patients with bullous pemphigoid) and C3c deposition using an indirect immunofluorescence (IIF) assay on monkey esophagus substrate.

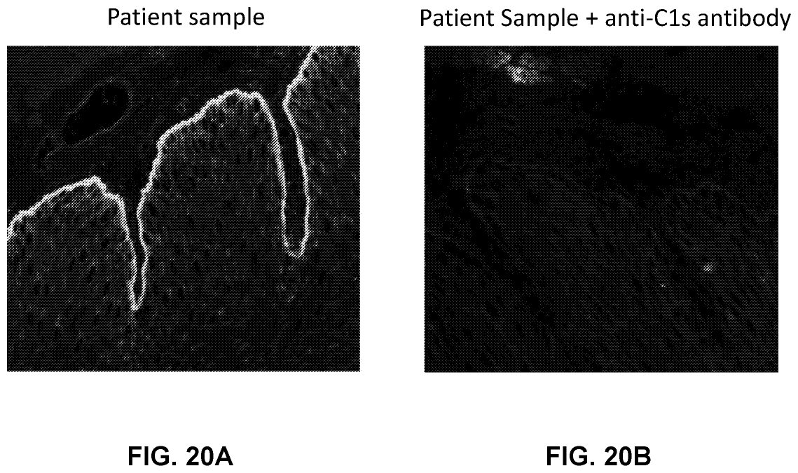

Briefly, patient samples were incubated with monkey esophagus substrate according to known techniques. The samples were then fluorescently stained for the presence of complement component C3c and visualized under a fluorescent microscope. Positive fluorescent staining indicates C3c is deposited on the cell surface. The results are shown in FIG. 20A-20B.

19 out of 47 samples (40%) demonstrated positive C3c staining. As shown in FIG. 20A, C3c deposition on monkey esophageal tissue was detected when sera from BP patients was incubated with the monkey esophagus tissue sections. However, incubating the same serum sample with an anti-C1s antibody comprising the variable light chain sequence set forth in SEQ ID NO: 7 and the variable heavy chain sequence set forth in SEQ ID NO: 8 resulted in near complete inhibition of C3c deposition as measured by IIF on monkey esophagus (FIG. 20B).

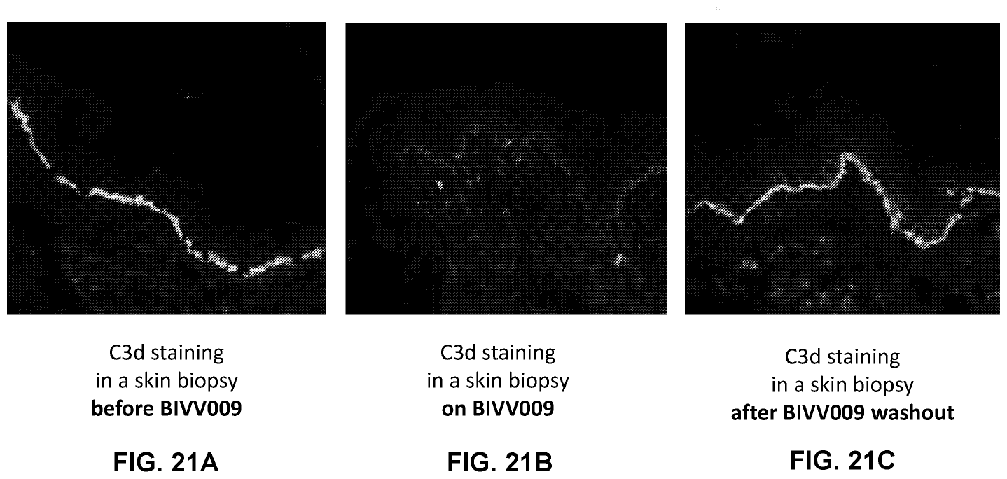

A second experiment showed that this antibody inhibits deposition of complement component C3d at the dermal-epidermal junction in human biopsy samples. As part of a Phase 1b trial (described below in Example 9), bullous pemphigoid patients were treated with the anti-C1s BIVV009 antibody. Eight patients consented to skin biopsies taken before and during BIVV009 treatment. Of the eight patients, five patients presented with complement C3d deposition at the dermal-epidermal junction before beginning treatment with BIVV009 (FIG. 21A). During BIVV009 treatment, however, the C3d fluorescent staining was nearly completely abolished, showing that BIVV009 inhibited C3d complement deposition (FIG. 21B). Following BIVV009 treatment, the C3d fluorescent staining returned, indicating C3d deposition resolution. (FIG. 21C).

### Example 9

### Treatment of Patients with Moderate to Severe Bullous Pemphigoid

An effective dose of an anti-C1s antibody (e.g., BIVV009) will be administered to subjects having BP. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 10

### Treatment of Multifocal Motor Neuropathy (MMN)

An effective dose of an anti-C1s antibody (e.g., BIVV009) will be administered to subjects having MMN. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 11

### Treatment of Chronic Inflammatory Demyelinating Polyneuropathy (CIDP)

An effective dose of an anti-C1s antibody (e.g., BIVV009) will be administered to subjects having CIDP. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 12

### Treatment of Myasthenia Gravis (MG)

An effective dose of an anti-C1s antibody (e.g., BIVV009) will be administered to subjects having MG. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 13

### Treatment of Neuromyeltisi Optica (NMO)

An effective dose of an anti-C1s antibody (e.g., BIVV009) will be administered to subjects having NMO. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 14

### Treatment of Systemic Lupus Erythematosus (SLE)

An effective dose of an anti-C1s antibody (e.g., BIVV009) will be administered to subjects having SLE. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 15

### Treatment of Lupus Nephritis (LN)

An effective dose of an anti-C1s antibody (e.g., BIVV009) will be administered to subjects having LN. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

### Example 16

### Treatment of Membranoproliferative Glomerulonephritis (MPGN)

An effective dose of an anti-C1s antibody (e.g., BIVV009) will be administered to subjects having MPGN. An effective dose of the anti-C1s antibody (e.g., BIVV009) dose will be either 6.5 gram dose (in patients <75 kg) or a 7.5 gram dose (in patients ≥75 kg), depending on the subjects' body weight at Baseline (Day 0). The subjects can receive a single dose or multiple doses, depending on the progress of the treatment. When multiple doses are given, the dosing interval can be two weeks (once every two week administration).

While the present disclosure has been described with reference to the specific embodiments thereof, it should be understood by those skilled in the art that various changes can be made and equivalents can be substituted without departing from the true spirit and scope of the disclosure. In addition, many modifications can be made to adapt a particular situation, material, composition of matter, process, process step or steps, to the objective, spirit and scope of the present disclosure. All such modifications are intended to be within the scope of the claims appended hereto.

All publications, patents, and patent applications disclosed herein are incorporated by reference to the same extent as if each individual publication, patent or patent application was specifically and individually indicated to be incorporated by reference.

1.-15. (canceled)

16. A method comprising administering to a subject having a complement-mediated disorder an effective dose of a humanized monoclonal anti-C1s antibody that comprises light chain complementarity determining regions (CDRs) of an antibody light chain variable region comprising the amino acid sequence of SEQ ID NO: 7 and heavy chain CDRs of an antibody heavy chain variable region comprising the amino acid sequence of SEQ ID NO: 8, wherein the effective dose is 5 grams to 8 grams of the humanized anti-C1s antibody.

17. The method of claim 16, wherein the humanized monoclonal anti-C1s antibody comprises a light chain complementarity determining region 1 (CDR1) comprising the amino acid sequence of SEQ ID NO: 10, a light chain complementarity determining region 2 (CDR2) comprising the amino acid sequence of SEQ ID NO: 11, and a light chain complementarity determining region 3 (CDR3) comprising the amino acid sequence of SEQ ID NO: 3, a heavy chain CDR1 comprising the amino acid sequence of SEQ ID NO: 12, a heavy chain CDR2 comprising the amino acid sequence of SEQ ID NO: 13, a heavy chain CDR3 comprising the amino acid sequence of SEQ ID NO: 14.

18. The method of claim 16, wherein the humanized monoclonal anti-C1s antibody comprises a light chain variable region comprising the amino acid sequence of SEQ ID NO: 16 and a heavy chain variable region comprising the amino acid sequence of SEQ ID NO: 21.

19. The method of claim 18, wherein the humanized monoclonal anti-C1s antibody comprises a light chain that comprises the amino acid sequence of SEQ ID NO: 23 and a heavy chain that comprises the amino acid sequence of SEQ ID NO: 22.

20. The method of claim 16, wherein the complement-mediated disorder is selected from the group consisting of: cold agglutinin disease, antibody-mediated kidney allograft rejection, immune thrombocytopenia, bullous pemphigoid, multifocal motor neuropathy, chronic inflammatory demyelinating polyneuropathy, myasthenia gravis, neuromyelitis optica, systemic lupus erythematosus, lupus nephritis, and membranoproliferative glomerulonephritis.

21. The method of claim 20, wherein the complement mediated disorder is cold agglutinin disease.

22. The method of claim 21, wherein the subject has not had a blood transfusion within 6 months of administering the effective dose.

23. The method of claim 21, wherein the subject has had a blood transfusion within 6 months of administering the effective dose.

24. The method of claim 16, wherein the level of hemoglobin in the subject is increased by at least 1.6 g/dL within seven days of administering the effective dose, relative to baseline levels of hemoglobin in the subject.

25. The method of claim 16, wherein the level of bilirubin in the subject is lower than 1.2 g/dL within seven days of administering the effective dose.

26. The method of claim 16, wherein the level of haptoglobin, the level of lactate dehydrogenase, and/or the level of reticulocytes is/are normalized in the subject following repeat administration of the effective dose.

27. The method of claim 20, wherein the complement mediated disorder is immune thrombocytopenia.

28. The method of claim 27, wherein the complement mediated disorder is chronic immune thrombocytopenia.

29. The method of claim 16, wherein following administration of the effective dose the subject has a serum concentration of the humanized monoclonal anti-C1s antibody of at least 100 g/mL.

30. The method of claim 16, wherein the effective dose is 6.5 grams to 7.5 grams.

31. The method of claim 30, wherein the effective dose is 6.5 grams.

32. The method of claim 31, wherein the subject weighs less than 75 kilograms.

33. The method of claim 30, wherein the effective dose is 7.5 grams.

34. The method of claim 33, wherein the subject weighs 75 kilograms or more.

35. The method of claim 16, wherein the effective dose of the humanized monoclonal anti-C1s antibody is administered intravenously, subcutaneously, or intramuscularly.

36. A method comprising intravenously administering to a subject having cold agglutinin disease an effective dose of a humanized monoclonal anti-C1s antibody that comprises a light chain comprising the amino acid sequence of SEQ ID NO: 23 and a heavy chain comprising the amino acid sequence of SEQ ID NO: 22, wherein: (a) the subject weighs less than 75 kilograms, and the effective dose is 6.5 grams of the humanized anti-C1s antibody; or (b) the subject weighs 75 kilograms or more, and the effective dose is 7.5 grams of the humanized anti-C1s antibody.

37. The method of claim 16, wherein the effective dose is administered by intravenous infusion over 1 hour.

38. The method of claim 36, wherein the effective dose is administered on Day 0, Day 7, and every 14 days thereafter starting on Day 21.

39. A humanized monoclonal anti-C1s antibody comprising a light chain that comprises the amino acid sequence of SEQ ID NO: 23 and a heavy chain that comprises the amino acid sequence of SEQ ID NO: 22.

40. A pharmaceutical composition for treating a complement-mediated disorder in a subject, the pharmaceutical composition comprising the humanized monoclonal anti-C1s antibody of claim 39.

41. The pharmaceutical composition of claim 40, wherein the humanized monoclonal anti-C1s antibody is formulated as a solution at a concentration of 50 mg/mL.

42. A pharmaceutical composition for treating a complement-mediated disorder in a subject, the pharmaceutical composition comprising an effective dose of a humanized monoclonal anti-C1s antibody that comprises light chain complementarity determining regions (CDRs) of an antibody light chain variable region comprising the amino acid sequence of SEQ ID NO: 7 and heavy chain CDRs of an antibody heavy chain variable region comprising the amino acid sequence of SEQ ID NO: 8, wherein the effective dose is 5 grams to 8 grams of the humanized anti-C1s antibody.

43. The pharmaceutical composition of claim 42, wherein the humanized monoclonal anti-C1s antibody comprises a light chain complementarity determining region 1 (CDR1) comprising the amino acid sequence of SEQ ID NO: 10, a light chain complementarity determining region 2 (CDR2) comprising the amino acid sequence of SEQ ID NO: 11, and a light chain complementarity determining region 3 (CDR3) comprising the amino acid sequence of SEQ ID NO: 3, a heavy chain CDR1 comprising the amino acid sequence of SEQ ID NO: 12, a heavy chain CDR2 comprising the amino acid sequence of SEQ ID NO: 13, a heavy chain CDR3 comprising the amino acid sequence of SEQ ID NO: 14.

44. The pharmaceutical composition of claim 43, wherein the humanized monoclonal anti-C1s antibody comprises a light chain variable region comprising the amino acid sequence of SEQ ID NO: 16 and a heavy chain variable region comprising the amino acid sequence of SEQ ID NO: 21.

45. The pharmaceutical composition of claim 44, wherein the humanized monoclonal anti-C1s antibody comprises a light chain that comprises the amino acid sequence of SEQ ID NO: 23 and a heavy chain that comprises the amino acid sequence of SEQ ID NO: 22.

46. The pharmaceutical composition of claim 42, wherein the effective dose is 6.5 grams to 7.5 grams.

47. The pharmaceutical composition of claim 46, wherein the effective dose is 6.5 grams.

48. The pharmaceutical composition of claim 47, wherein the subject weighs less than 75 kilograms.

49. The pharmaceutical composition of claim 46, wherein the effective dose is 7.5 grams.

50. The pharmaceutical composition of claim 49, wherein the subject weighs 75 kilograms or more.

51. The pharmaceutical composition of claim 40, further comprising sterile saline.

52. The pharmaceutical composition of claim 40, wherein the complement-mediated disorder is selected from the group consisting of: cold agglutinin disease, antibody-mediated kidney allograft rejection, immune thrombocytopenia, bullous pemphigoid, multifocal motor neuropathy, chronic inflammatory demyelinating polyneuropathy, myasthenia gravis, neuromyelitis optica, systemic lupus erythematosus, lupus nephritis, and membranoproliferative glomerulonephritis.

53. The pharmaceutical composition of claim 52, wherein the complement mediated disorder is cold agglutinin disease.

54. The pharmaceutical composition of claim 52, wherein the complement mediated disorder is immune thrombocytopenia.

55. The pharmaceutical composition of claim 54, wherein the complement mediated disorder is chronic immune thrombocytopenia.


SEQUENCE LISTING (23 sequences)

SEQ ID NO: 1 - protein, 12 aa, Artificial Sequence
  a light chain region comprising CDR-L1
SSVSSSYLHWYQ

SEQ ID NO: 2 - protein, 10 aa, Artificial Sequence
  a light chain region comprising CDR-L2
STSNLASGVP

SEQ ID NO: 3 - protein, 10 aa, Artificial Sequence
  a light chain region comprising CDR-L3
HQYYRLPPIT

SEQ ID NO: 4 - protein, 12 aa, Artificial Sequence
  a heavy chain region comprising CDR-H1
GFTFSNYAMSWV

SEQ ID NO: 5 - protein, 10 aa, Artificial Sequence
  a heavy chain region comprising CDR-H2
ISSGGSHTYY

SEQ ID NO: 6 - protein, 11 aa, Artificial Sequence
  a heavy chain region comprising CDR-H3
ARLFTGYAMDY

SEQ ID NO: 7 - protein, 109 aa, Artificial Sequence
  VL region
QIVLTQSPAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 8 - protein, 118 aa, Artificial Sequence
  VH region
EVMLVESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVTVSS

SEQ ID NO: 9 - protein, 673 aa, Artificial Sequence
  amino acid sequence of human C1s
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 10 - protein, 12 aa, Artificial Sequence
  a light chain region comprising CDR-L1
TASSSVSSSYLH

SEQ ID NO: 11 - protein, 7 aa, Artificial Sequence
  a light chain region comprising CDR-L2
STSNLAS

SEQ ID NO: 12 - protein, 5 aa, Artificial Sequence
  a heavy chain region comprising CDR-H1
NYAMS

SEQ ID NO: 13 - protein, 17 aa, Artificial Sequence
  a heavy chain region comprising CDR-H2
TISSGGSHTYYLDSVKG

SEQ ID NO: 14 - protein, 9 aa, Artificial Sequence
  a heavy chain region comprising CDR-H3
LFTGYAMDY

SEQ ID NO: 15 - protein, 109 aa, Artificial Sequence
  VL region
QIVLTQSPAILSLSPGERATMSCTASSSVSSSYLHWYQQKPGKAPKLWIYSTSNLASGVPSRFSGSGSGTFYTLTISSLQAEDFATYYCHQYYRLPPITFGQGTKLEIK

SEQ ID NO: 16 - protein, 109 aa, Artificial Sequence
  VL region
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 17 - protein, 109 aa, Artificial Sequence
  VL region
QIVLTQSPATLSLSPGERATLSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 18 - protein, 118 aa, Artificial Sequence
  VH region
EVMLVESGGGLVKPGGSLRLSCAASGFTFSNYAMSWVRQAPGKGLEWVATISSGGSHTYYLDSVKGRFTISRDNSKDTLYLQMSSLRAEDTALYYCARLFTGYAMDYWGQGTSVTVSS

SEQ ID NO: 19 - protein, 118 aa, Artificial Sequence
  VH region
EVMLVESGGGLVKPGGSLRLSCAASGFTFSNYAMSWVRQAPGKGLEWVATISSGGSHTYYLDSVKGRFTISRDNSKDTLYLQMNSLRAEDTALYYCARLFTGYAMDYWGQGTLVTVSS

SEQ ID NO: 20 - protein, 118 aa, Artificial Sequence; identical to SEQ ID NO: 18
  VH region

SEQ ID NO: 21 - protein, 118 aa, Artificial Sequence
  VH region
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSS

SEQ ID NO: 22 - protein, 445 aa, Artificial Sequence
  Heavy Chain of BIVV009
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 216 aa, Artificial Sequence
  Light Chain of BIVV009
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC